(19) United States
(12) Patent Application Publication
(10) Pub. No.: US 2024/0391946 A1
(43) Pub. Date: Nov. 28, 2024

(54) COMPOSITIONS, SYSTEMS, AND METHODS FOR NUCLEIC ACID DATA STORAGE

(71) Applicant: NAIO, Inc., San Carlos, CA (US)

(72) Inventor: Eric Kool, Stanford, CA (US)

(21) Appl. No.: 18/410,091

(22) Filed: Jan. 11, 2024

Related U.S. Application Data
(63) Continuation of application No. PCT/US2022/042660, filed on Sep. 6, 2022.
(60) Provisional application No. 63/241,479, filed on Sep. 7, 2021.
(60) Provisional application No. 63/262,686, filed on Oct. 18, 2021.

(51) Int. Cl. C07H 21/04; C07H 99/00

(52) U.S. Cl. CPC C07H 21/04; C07H 99/00

(57) ABSTRACT

Described herein are writable polymers for data storage and related methods. Generally, a writable polymer may contain one or more convertible residues (e.g., convertible residues comprising a modifiable fluorophore with switchable fluorescent states) that are enabled to provide a data code. Various methods can be utilized to generate a writable polymer (e.g., a writable nucleic polymer). Various methods can be utilized to encode a writable polymer by selectively modifying the one or more convertible residues (e.g., modifiable fluorophores). Various methods of reading a polymer encoded with data are also described herein.

### CROSS REFERENCE TO RELATED APPLICATIONS

This application is a continuation application of International Patent Application No. PCT/US2022/042660, filed Sep. 6, 2022, which claims the benefit of U.S. Provisional Patent Application No. 63/241,479, filed on Sep. 7, 2021, and U.S. Provisional Patent Application No. 63/262,686, filed on Oct. 18, 2021, the content of each of which is incorporated by reference herein in its entirety.

### SEQUENCE LISTING

The instant application contains a Sequence Listing which has been submitted electronically in XML file format and is hereby incorporated by reference in its entirety. Said XML copy, created on Feb. 15, 2024, is named 63281-703.301.xml and is 9,647 bytes in size.

### BACKGROUND

As the amount of digital data increases worldwide, the complications of storing digital data long term are becoming a rapidly growing issue. Electronically or magnetically archived digital data can easily be manipulated, distorted, and/or lost while in storage. While efficient solid-state electronic methods for archival data storage exist, it is not stable over a period of years, resulting in loss of data unless the data is periodically rewritten or transferred to a new device. Similarly, magnetic tape is commonly used for data archiving, but it also degrades over time. Therefore, ways to efficiently encode and store data, especially over long periods, are being pursued very actively.

Polymers (including nucleic acid molecules) offer a potential solution for overcoming issues with data storage. With their sequences of repeated monomers, polymers can be utilized as molecules containing digital information, which can be stably stored at high densities for extremely long durations in time. For instance, natural DNA contains digital information encoded in the four bases: A, C, T, and G, and can be used to encode binary data in its sequence in synthesized strands. A single polymeric molecule of DNA can be very long (such as in chromosomes) and can encode millions of bits of data. It has been estimated that 1 cubic inch of DNA can encode 10¹⁸ bytes of data. Furthermore, DNA is relatively stable, and has yielded sequence information even from samples tens of thousands of years old. Thus, DNA offers considerable promise for archiving data. Other polymers can be similarly stable over time as well.

The use of polymeric molecules to encode digital data is of interest in research. In principle, a sequence of monomers or iteratively spaced functional groups attached to select monomers can encode ones and zeros in the binary sense. Such molecular data encoding offers the possibility of very high data density, since a single molecule can encode many bits of data. Moreover, some polymers (such as DNA) can remain stable longer than electronic or magnetic media, thus obviating the need for frequent re-writing of data for archival storage.

Although nucleic acids are a great potential source of data storage, the process of synthesizing of nucleic acids in particular data-defining sequences is inefficient and thus the process of encoding the nucleic acids is a substantial barrier to utilizing nucleic acids as data storage. Current approaches for storing data in DNA involve chemical or enzymatic synthesis of strands of arbitrary sequences that encode digital information (see G. M. Church, Y. Gao, and S. Kosuri Science. 2012; 337:1628; X. Chengtao, et al., Nucleic Acids Res. 2021; 49:5451-5469; and E. Yoo, et al., Comput Struct Biotechnol J. 2021; 19:2468-2476; the disclosures of which are each incorporated herein by reference). Oligonucleotide synthesizers can produce DNAs of length up to roughly 100-200 nucleotides. Specialized synthesizers can produce hundreds or thousands of oligonucleotides at one time, which promises higher throughput of data writing. In addition to chemical DNA synthesis, enzymatic approaches involving polymerases or other enzymes are also under investigation for creating DNAs of arbitrary data-encoding sequence. These involve adding specialized nucleotides (or small groups of nucleotides) one at a time, or short segments of DNA step by step.

Because of the well-developed methods for synthesis and analysis of DNA, this biopolymer has received by far the most attention in molecular data encoding studies. The approach of encoding data in DNA during synthesis is limited by yield, strand length, time, and cost. Current efficient DNA synthesizers produce strands up to roughly 200 nucleotides, and thus encode relatively small amounts of information. Large numbers of different oligonucleotides must be synthesized to compensate for the short sequences. Oligonucleotide synthesis requires excess reagents to achieve high stepwise yields and requires expensive consumption of reagents and solvents. It also requires time to achieve these high yields for each nucleotide addition (commonly 1-5 min for each step), which implies the need for extended time for encoding larger amounts of data. Common enzymatic approaches under development similarly add nucleotides or groups of nucleotides in stepwise fashion and have not yet greatly improved on the ability to produce very long strands and encode large amounts of data. Because the enzymatic approaches also occur stepwise, they also have limits in the speed of data encoding. Further, since both the above chemical and enzymatic strategies typically produce relatively short strands, they may not be ideal for single molecule sequencing, and instead may rely on sequencing methods that require larger amounts of each written DNA. Ideally, data would be encoded in very long DNA polymers (for instance, greater than 10 kilobases), but methods for making very long DNAs of arbitrary sequence are limited or nonexistent. Biologically synthesized DNAs, while very long, do not exist in arbitrary sequence, limiting the ability to encode data efficiently in these polymers.

Non-DNA (e.g., organic) polymers also contain many monomers and might also have the potential to store data. However, polymers are usually synthesized (polymerized) in one step, without the ability to control the sequence of monomers contained in a molecular strand, thus making it difficult to encode data based on sequence. If synthesized step by step (such as peptide synthesis), polymers also would be quite limited in length and would require significant time for each step. For these reasons, improved solutions are needed for encoding data in polymers.

### SUMMARY

In one aspect, provided here are polymers for encoding data, comprising:
a plurality of convertible residues iteratively spaced along and covalently linked to the backbone of the polymer,
wherein each convertible residue of the plurality of convertible residues has a first fluorescent state, and is capable of being converted from the first fluorescent state into a second fluorescent state, the first fluorescent state and the second fluorescent state being different;
wherein each of all or a subset of the plurality of convertible residues comprises a fluorophore, and wherein the plurality of convertible residues is covalently linked to the polymer in the first fluorescent state and in the second fluorescent state.

In certain embodiments, the fluorophores are modifiable fluorophores.

In certain embodiments, the fluorophore is modifiable by light.

In certain embodiments, each fluorophore of the subset of convertible residues is iteratively spaced along the polymer and linked via the backbone.

In certain embodiments, the polymer further comprises a plurality of charged constituents iteratively spaced along the polymer.

In certain embodiments, the plurality of modifiable fluorophores comprises caged fluorophores that are capable of being converted to uncaged fluorophores by light.

In certain embodiments, each of the caged fluorophores is selected from

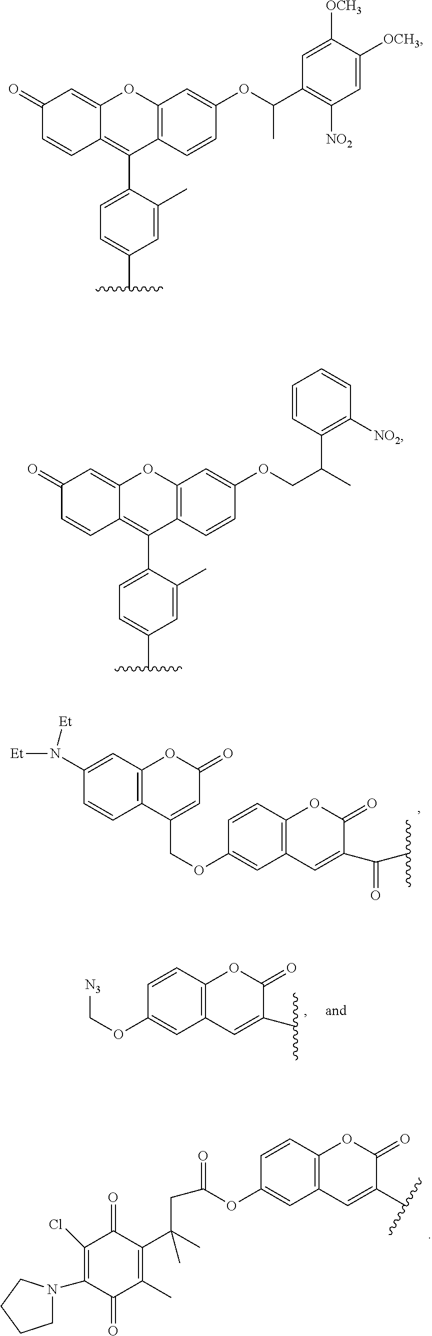 [SMILES: *c1ccc(-c2c3ccc(=O)cc-3oc3cc(OC(C)c4cc(OC)c(OC)cc4[N+](=O)[O-])ccc23)c(C)c1 ; *c1ccc(-c2c3ccc(=O)cc-3oc3cc(OCC(C)c4ccccc4[N+](=O)[O-])ccc23)c(C)c1 ; *C(=O)c1cc2cc(OCc3cc(=O)oc4cc(N(CC)CC)ccc34)ccc2oc1=O ; *c1cc2cc(OCN=[N+]=[N-])ccc2oc1=O ; *c1cc2cc(OC(=O)CC(C)(C)C3=C(C)C(=O)C(N4CCCC4)=C(Cl)C3=O)ccc2oc1=O]

In certain embodiments the fluorophores are in proximity of a releasable quencher, wherein the quencher is capable of being released by light and can quench fluorescence upon release.

In certain embodiments, each convertible residue of a subset of the plurality of convertible residues comprises the quencher.

In certain embodiments, the quencher is selected from

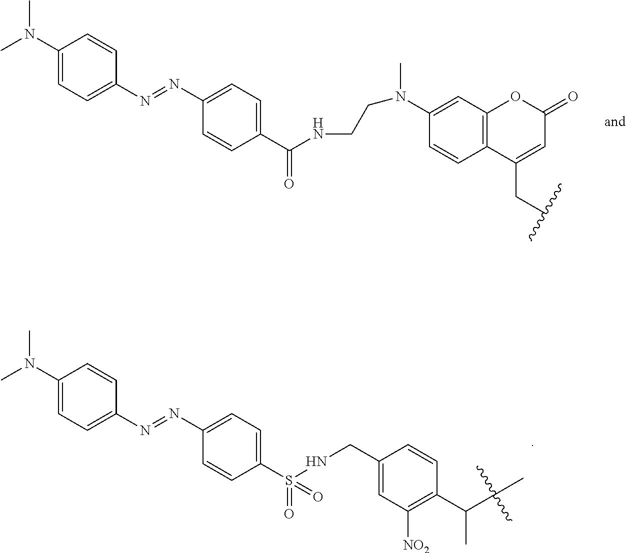 [SMILES: *Cc1cc(=O)oc2cc(N(C)CCNC(=O)c3ccc(/N=N/c4ccc(N(C)C)cc4)cc3)ccc12 ; *C(C)c1ccc(CNS(=O)(=O)c2ccc(/N=N/c3ccc(N(C)C)cc3)cc2)cc1[N+](=O)[O-]]

In certain embodiments, the modifiable fluorophores comprise photoconvertible fluorophores, wherein the photoconvertible fluorophores exist in a first structural state having a first emission wavelength and are capable of being converted into a second structural state having a second emission wavelength via the light pulses.

In certain embodiments, the conversion of the photoconvertible fluorophores from the first structural state into a second structural state is via light pulses at a first wavelength; and wherein the photoconvertible fluorophores are capable of being converted into a third structural state having a third emission wavelength via light pulses at a second wavelength.

In certain embodiments, the photoconvertible fluorophores are activated by light.

In certain embodiments, photoconvertible fluorophores are activated by light in the presence of an additive.

In certain embodiments, the photoconvertible fluorophores are inactivated by light.

In certain embodiments, the photoconvertible fluorophores are inactivated by light in the presence of an additive.

In certain embodiments, the photoconvertible fluorophore comprises a polymethine cyanine dye.

In certain embodiments, the photoconvertible fluorophore comprises

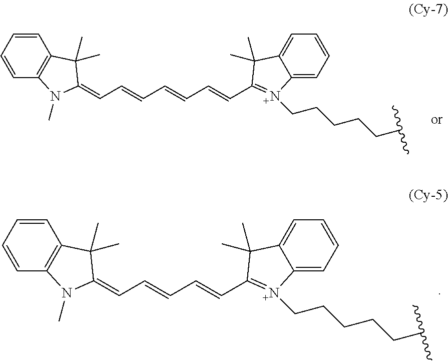 [SMILES: *CCCCC[N+]1=C(/C=C/C=C/C=C/C=C2/N(C)c3ccccc3C2(C)C)C(C)(C)c2ccccc21 ; *CCCCC[N+]1=C(/C=C/C=C/C=C2/N(C)c3ccccc3C2(C)C)C(C)(C)c2ccccc21]

In certain embodiments, the plurality of modifiable fluorophores comprise releasable fluorophores that are capable of being released from the polymer by light.

In certain embodiments, the plurality of modifiable fluorophores comprise photobleachable fluorophores that are capable of being bleached by light.

In certain embodiments, the modifiable fluorophores comprise a plurality of pairs of constitutive fluorophores and caged fluorophores within Forster resonance energy transfer distance; wherein the plurality of pairs of constitutive fluorophores and caged fluorophores are iteratively spaced along the polymer; and wherein the caged fluorophores are capable of being converted to uncaged fluorophores by light.

In certain embodiments, the modifiable fluorophores comprise a plurality of pairs of constitutive fluorophores and quenched fluorophores within Forster resonance energy transfer distance; wherein the plurality of pairs of constitutive fluorophores and quenched fluorophores are iteratively spaced along the polymer; and wherein each quenched fluorophore is in proximity of quencher that is capable of being released via light pulses.

In certain embodiments, the modifiable fluorophores comprises a plurality of pairs of two caged fluorophores within Forster resonance energy transfer distance; wherein the plurality of pairs of two caged fluorophores are iteratively spaced along the polymer; wherein each caged fluorophore of the pairs is capable of being uncaged via light pulses; and wherein a first caged fluorophore of the pairs is capable of emitting fluorescence at a first emission wavelength when uncaged and a second caged fluorophore of the pairs is capable of emitting fluorescence at a second emission wavelength when uncaged.

In certain embodiments, the first caged fluorophore of the pairs is uncaged via light pulses at a first wavelength and the second caged fluorophore of the pairs is uncaged via light pulses at a second wavelength.

In certain embodiments, the plurality of modifiable fluorophores comprises a plurality of pairs of two quenched fluorophores within Förster resonant energy transfer distance; wherein the plurality of pairs of two quenched fluorophores are iteratively spaced along the polymer; wherein each quenched fluorophore of the pairs is in proximity of a quencher that is capable of being released via light pulses; and wherein a first quenched fluorophore of the pairs is capable of emitting fluorescence at the first emission wavelength when its proximate quencher is released and a second quenched fluorophore of the pairs is capable of emitting fluorescence at the second emission wavelength when its proximate quencher is released.

In certain embodiments, the first quenched fluorophore of the pairs is in proximity of a quencher that is releasable via light pulses at the first wavelength and the second quenched fluorophore of the pairs is in proximity of a quencher that is releasable via light pulses at the second wavelength.

In certain embodiments, the plurality of modifiable fluorophores comprises a plurality of pairs of fluorophores within Förster resonant energy transfer distance; wherein a first fluorophore of the pairs is caged and capable of being uncaged via light pulses; wherein a second fluorophore of the pairs is a quenched fluorophore in proximity of a quencher that is capable of being released via light pulses; wherein the plurality of pairs of the two fluorophores are iteratively spaced along the polymer; and wherein the caged fluorophore of the pairs is capable of emitting fluorescence at a first wavelength when uncaged and the quenched fluorophore of the pairs is capable of emitting fluorescence at a second wavelength when its proximate quencher is released.

In certain embodiments, the first caged fluorophore of the pairs is uncaged via light pulses at the first wavelength and the second quenched fluorophore of the pairs is in proximity of a quencher that is releasable via light pulses at the pulse wavelength.

In certain embodiments, the first caged fluorophore of the pairs is uncaged via light pulses at the first wavelength, or a second wavelength and the second quenched fluorophore of the pairs is in proximity of a quencher that is releasable via light pulses at the first wavelength.

In certain embodiments, the polymer is a biological polymer.

In certain embodiments, the polymer is a nucleic acid polymer, and the plurality of convertible residues are convertible nucleobases.

In certain embodiments, the nucleic acid polymer is a single-stranded nucleic acid polymer.

In certain embodiments, the nucleic acid polymer is double-stranded nucleic acid polymer.

In certain embodiments, the nucleic acid polymer comprises Deoxyribonucleic acid (DNA), Ribonucleic acid (RNA), 2′-O-alkyl RNA, phosphorothioate DNA, glycerol nucleic acids (GNA), threose nucleic acids (TNA), locked nucleic acids (LNA), or a combination thereof.

In certain embodiments, the nucleic acid polymer comprises greater than 10 convertible residues.

In certain embodiments, the ratio of the total number of nucleotides to the convertible residues in the nucleic acid polymer is between 2 to 1000.

In certain embodiments, the ratio of the total number of nucleotides to the convertible residues in the nucleic acid polymer is between 2 to 100.

In certain embodiments, the ratio of the total number of nucleotides to the convertible residues in the nucleic acid polymer is between 3 to 100.

In certain embodiments, the plurality of convertible nucleobases are non-naturally occurring nucleobases.

In certain embodiments, the plurality of convertible nucleobases are modified naturally occurring nucleobases or derivatives of naturally occurring nucleobases.

In certain embodiments, the plurality of convertible residues in the first fluorescent state and the second fluorescent state are readable by polymerase.

In certain embodiments, the polymer is an organic polymer.

In certain embodiments, the fluorophore is directly attached to the base of the convertible nucleobases.

In certain embodiments, the fluorophore is attached to the base of the nucleobase with a linker.

In certain embodiments, the fluorophore is attached to the base of the nucleobase without a linker or linker atom.

In certain embodiments, the plurality of convertible residues comprise fluorescent nucleobases.

In certain embodiments, the fluorophore is a nucleobase.

In certain embodiments, the nucleobase is a fluorescent nucleobase.

In certain embodiments, the plurality of convertible nucleobases is covalently linked to the backbone of the nucleic acid via the sugar.

In certain embodiments, the fluorophore is activatable by light, voltage, enzymatic agent, chemical reagent, or a redox agent, thereby converting from the first fluorescent state into the second fluorescent state.

In certain embodiments, the fluorophore is activatable by light, thereby converting from the first fluorescent state into the second fluorescent state.

In certain embodiments, the conversion from the first fluorescent state into the second fluorescent state occurs via an irreversible reaction.

In certain embodiments, the backbone of the polymer (e.g., phosphate and sugar in nucleic acid polymer) remain unchanged during the conversion from the first fluorescent state into the second fluorescent state.

In certain embodiments, the polymer comprises two or more different sets of convertible residues, each set of convertible residues has a first fluorescent state and is capable of being converted from the first fluorescent state into a second fluorescent state, the first fluorescent state and the second fluorescent state being different.

In certain embodiments, the two or more different sets of convertible residues are activatable by light of a different wavelength.

In certain embodiments, a first set of convertible residues is activatable by light of a first wavelength, and a second set of convertible residues is activatable by light of a second wavelength, the first wavelength and the second wavelength being different.

In certain embodiments, the fluorophore comprises one or more photo-removable or photo-cleavable groups.

In certain embodiments, the convertible residue comprises a leaving group.

In certain embodiments, the leaving group is a quencher and/or a cage (e.g., photo-removable or photo-cleavable group).

In certain embodiments, the plurality of convertible residues are capable of being converted by light of a wavelength of 325 nm, 360 nm, 400 nm, 500 nm, 600 nm, 700 nm, or 800 nm.

In certain embodiments, the first fluorescent state and the second fluorescent state of the plurality of convertible residues are readable by a fluorescence detection device.

In certain embodiments, the fluorescence detection device is a fluorescence plate reader, gel imager, fluorescence spectrometer, fluorescence microscope, or a flow cytometer.

In certain embodiments, the first fluorescent state and the second fluorescent state of the plurality of convertible residues are readable by a sequencing method capable of detecting and differentiating non-naturally occurring and/or modified nucleobases.

In certain embodiments, when the plurality of convertible residues are converted to the second fluorescent state, properties of the plurality of convertible residues are modified (e.g., having reduced size, altered shape, modified H-bonding, and/or modified polymerase substrate ability) as compared to the first fluorescent state.

In certain embodiments, the first fluorescent state and the second fluorescent state of the plurality of convertible residues are readable by nanopore sequencing.

In certain embodiments, one or more of the plurality of convertible residues are capable of being converted from the second fluorescent state into a third fluorescent state; wherein the one or more of the plurality of convertible residues are attached covalently to the nucleic acid polymer in the third fluorescent state.

In certain embodiments, each of the plurality of convertible residues is capable of being independently and selectively converted.

In certain embodiments, the polymers provided herein further comprise a plurality of spacer residues linked via the backbone of the polymer, wherein each of the plurality of convertible residues are separated by one or more spacer residues of the plurality of spacer residues.

In certain embodiments, the iterative spacing among the plurality of convertible residues conforms to a resolution of a writing mechanism for encoding data on the polymer.

In certain embodiments, the iterative spacing among two adjacent convertible residues is equal to or greater than a resolution of a data encoding mechanism for encoding data into the polymer.

In certain embodiments, the plurality of spacer residues does not interfere with reading of the convertible residues.

In certain embodiments, the plurality of spacer residues in the polymer are the same spacer residues.

In certain embodiments, the plurality of spacer residues comprises two or more different spacer residues (e.g., different nucleobases such as different naturally occurring nucleobases).

In certain embodiments, the polymer consists essentially of spacer residues.

In certain embodiments, each of the plurality of convertible residues are separated by 2, 3, 4, 5, 6, 7, 8, 9, 10, 20, 30, 40, or 50 spacer residues.

In certain embodiments, each of the plurality of convertible residues are separated by 6 spacer residues.

In certain embodiments, the plurality of spacer residues comprise naturally occurring nucleobases, non-naturally nucleobases, tetrahydrofuran abasic residues, or ethylene glycol residues.

In certain embodiments, the plurality of spacer residues comprise naturally occurring nucleobases.

In certain embodiments, the polymers provided herein further comprise one or more delimiters linked to the backbone of the polymer.

In certain embodiments, each of the one or more delimiters comprises one or more naturally occurring nucleobases or non-naturally nucleobases.

In certain embodiments, the one or more delimiters comprise naturally occurring nucleobases.

In certain embodiments, the one or more delimiters separate two or more adjacent data fields within the polymer.

In certain embodiments, the polymers provided herein further comprise one or more data tags.

In certain embodiments, the one or more data tags comprise one or more naturally occurring nucleobases or non-naturally nucleobases.

In certain embodiments, the polymer is a nucleic acid polymer and the one or more data tags are present at the 5′ or 3′ end of the nucleic acid polymer.

In certain embodiments, the one or more data tags are incorporated to the nucleic acid polymer during synthesis of the nucleic acid polymer, during conversion of the plurality of convertible residues to the second fluorescent state, or via ligation after the plurality of convertible residues are converted to the second fluorescent state.

In certain embodiments, the polymer can be stored under standard nucleic acid storage protocols.

In certain embodiments, the polymer is a nucleic acid polymer that can be stored in appropriate nuclease-free solution at room temperature, or at a lower temperature (e.g., −20° C.).

In certain embodiments, the polymer can be stored at room temperature without stabilizer.

In another aspect, also provided herein are systems for data writing, comprising:
a writable polymer comprising a plurality of convertible residues wherein each converted residue of the plurality comprises a first residue having a fluorophore, wherein each converted residue of the plurality is iteratively spaced along and covalently linked to the backbone of the polymer, wherein each of the plurality of convertible residues has a first fluorescent state and is capable of being converted from the first fluorescent state into a second fluorescent state, the first fluorescent state and the second fluorescent state being different; wherein the plurality of convertible residues are covalently linked to the polymer in the first fluorescent state and in the second fluorescent state; and
a data writing device for writing data on the writable polymer.

In certain embodiments, the writable polymer is a writable nucleic acid polymer and the plurality of convertible residues are convertible nucleobases.

In certain embodiments, the data writing device comprises a nanopore.

In certain embodiments, the data writing device converts the plurality of convertible residues into the second fluorescent state by light pulses, voltage pulses, an enzymatic agent, or a redox agent.

In certain embodiments, the data writing device converts the converts the plurality of convertible nucleobases residues into the second fluorescent state by light pulses.

In certain embodiments, the data writing device comprises a light irradiation device.

In yet another aspect, provided herein are methods for generating a writable nucleic acid polymer, comprising:
providing a circular single-stranded oligonucleotide template, wherein the circular single-stranded oligonucleotide template is complementary to a repeating data field that comprises a plurality of convertible nucleobases, wherein each convertible nucleobase of the plurality comprises a fluorophore; and
incubating the circular single-stranded oligonucleotide template in the presence of a nucleic acid primer, a polymerase, and triphosphate nucleotides, wherein the triphosphate nucleotides comprise a subset of convertible nucleobases of the plurality in a first fluorescent state and are capable of being converted from the first fluorescent state into a second fluorescent state, the first fluorescent state and the second fluorescent state being different.

In certain embodiments, the circular single-stranded oligonucleotide template comprises nucleobases complementary to the convertible nucleobases, and wherein the complementary nucleobases are iteratively spaced such that the incubation of the template with the nucleic acid primer, the polymerase, and the triphosphate nucleotides provides a nucleic acid polymer comprising a plurality of the convertible nucleobases iteratively spaced along and covalently linked via the backbone of the nucleic acid polymer; wherein the plurality of the convertible nucleobases are covalently linked to the nucleic acid polymer in the first fluorescent state and in the second fluorescent state.

In certain embodiments, the repeating data field further comprises spacer nucleobases, and wherein the triphosphate nucleotides further comprise triphosphate spacer nucleotides.

In yet another aspect, also provided herein are methods for generating a writable polymer, comprising:
chemically synthesizing a plurality of oligomers, each oligomer comprises a plurality of convertible residues, each convertible residue comprises a fluorophore, each convertible residue of the plurality iteratively spaced along and linked via the polymer backbone, wherein each of the plurality of convertible residues has a first fluorescent state and is capable of being converted from the first fluorescent state into a second fluorescent state; wherein the plurality of convertible residues are attached covalently to the polymer in the first fluorescent state and in the second fluorescent state, the first fluorescent state and the second fluorescent state being different; and
ligating the plurality of oligomers to form the writable polymer.

In certain embodiments, each of the plurality of oligomers comprises a plurality of spacer residues linked via the backbone of the nucleic acid polymer, wherein each of the plurality of the convertible residues is separated by one or more spacer residues of the plurality of spacer residues.

In certain embodiments, the ligating step is via chemical ligation.

In certain embodiments, the ligating step is via enzymatic ligation.

In certain embodiments, a complementary DNA splint is used in the ligating step.

In certain embodiments, the methods further comprises: annealing a plurality of complements to the oligomers prior to the ligating step.

In yet another aspect, provided herein are polymers encoded with data, comprising: a plurality of fluorophores iteratively spaced along the polymer and linked via a backbone, wherein the plurality of fluorophores comprises a plurality of modified fluorophores that have been modified from a first structural state into a second structural state via light pulses; and a plurality of charged constituents iteratively spaced along the polymer.

In certain embodiments, the plurality of fluorophores was incorporated into the polymer in the first structural state; and wherein the plurality of modified fluorophores was modified via device comprising a nanopore, the device is capable of traversing the polymer through the nanopore and selectively modifying the plurality of fluorophores vial the light pulses in accordance with a data code.

In certain embodiments, the plurality of modified fluorophores comprises uncaged fluorophores.

In certain embodiments, the plurality of modified fluorophores comprises unquenched fluorophores.

In certain embodiments, the plurality of modified residues comprises sites from which releasable fluorophores are released.

In certain embodiments, the plurality of modified fluorophores comprises fluorophores that have been inactivated or photobleached by light.

In certain embodiments, the plurality of modified fluorophores comprises photoconverted fluorophores, wherein the second structural state of the photoconverted fluorophores emits fluorescence at wavelength that is different than the wavelength emitted by the first structural state.

In yet another aspect, provided herein are methods of encoding data onto a writable polymer, comprising:
providing a writable polymer that comprises a plurality of convertible residues iteratively spaced along and linked to the polymer backbone, wherein each convertible residue of the plurality of convertible residues is provided having a first fluorescent state and is capable of being converted from the first fluorescent state into a second fluorescent state, the first fluorescent state and the second fluorescent state being different; wherein each of all or a subset of the plurality of convertible residues comprises a fluorophore, and wherein the plurality of convertible residues are covalently linked to the polymer in the first fluorescent state and in the second fluorescent state;
selectively modifying, utilizing a data writing device, a subset of the plurality of convertible residues into the second fluorescent state such that a data encoded polymer is generated.

In certain embodiments, the data writing device comprise a nanopore, and the method further comprising passing the writable nucleic acid polymer through the nanopore of the writing device, wherein the nanopore comprises a means to impinge light pulses onto the subset of the plurality of modifiable fluorophores into the second fluorescent state.

In certain embodiments, the plurality of modifiable fluorophores comprises caged fluorophores capable of being uncaged by light.

In certain embodiments, the plurality of modifiable fluorophores comprises fluorophores in proximity of a quencher, wherein the quencher is capable of being released by light.

In certain embodiments, the plurality of modifiable fluorophores comprises releasable fluorophores, wherein the releasable fluorophores are capable of being released by light.

In certain embodiments, the plurality of modifiable fluorophores comprises photoconvertible fluorophores, wherein the first structural state of the photoconvertible fluorophores has a first emission wavelength and the second structural state of the photoconvertible fluorophores has a second emission wavelength.

In certain embodiments, the selectively modifying, utilizing the data writing device, the subset of the plurality of convertible residues into the second fluorescent comprises uncaging of a caged fluorophore, releasing a quencher, releasing a fluorophore, photoinactivation of a fluorophore, photoactivation of fluorophore, bleaching of the fluorophore, or a combination thereof.

In certain embodiments, the uncaging of the caged fluorophore comprises releasing of a cage from the fluorophore.

In certain embodiments, the plurality of convertible residues are selectively modified in sequence.

In certain embodiments, the plurality of convertible residues are selectively modified in sequence from one end of the polymer to the other end of the polymer.

In certain embodiments, the method further comprises reading the encoded data of the generated polymer.

In certain embodiments, the encoded data is read by passing the generated polymer through the nanopore of a data reading unit, wherein the nanopore of the data reading unit comprises a detection module configured to detect fluorescence of each of the fluorescent states of the plurality of convertible residues.

In certain embodiments, the data reading unit comprises the same device as the data writing device.

In certain embodiments, the nanopore device to read data is the same nanopore device to write data.

In certain embodiments, the encoded data is read by stretching and imaging the generated polymer.

In certain embodiments, the encoded data on the polymer is read in sequence.

In certain embodiments, the encoded data on the polymer is read in sequence from one end of the polymer to the other end of the polymer.

In yet another aspect, also provided herein are methods for writing data onto a writable polymer, comprising:
providing a writable polymer that comprises a plurality of convertible residues iteratively spaced along and covalently linked via the backbone of the polymer, wherein each convertible residue of the plurality of convertible residues comprises a fluorophore, having a first fluorescent state and is capable of being converted from the first fluorescent state into a second fluorescent state, the first fluorescent state and the second fluorescent state being different; and
selectively converting, utilizing a data writing device, one or more of the pluralities of convertible residues into the second fluorescent state such that a data encoded polymer is generated.

In certain embodiments, the writable polymer is a writable nucleic acid polymer, and the plurality of convertible residues are convertible nucleobases.

In certain embodiments, the data writing device comprises a nanopore, and the method further comprising: passing the writable polymer through the nanopore of the writing device, wherein the nanopore comprises converts one or more of the pluralities of convertible residues into the second fluorescent state.

In certain embodiments, the nanopore is a plasmonic nanopore that provides light pulses or localized energy to selectively convert convertible nucleobases from the first fluorescent state into the second fluorescent state.

In certain embodiments, the data writing device comprises a plasmonic well or channel, and the method further comprising:
transferring the writable polymer into the plasmonic well or channel of the data encoding device, wherein the plasmonic well or channel provides light pulses or redox energy to selectively convert convertible nucleobases from the first fluorescent state into the second fluorescent state.

In certain embodiments, the data writing device selectively coverts the convertible residues into the second fluorescent state by light pulses, voltage pulses, an enzymatic agent, or a redox agent.

In certain embodiments, the data writing device selectively converts the converts the convertible residues into the second fluorescent state by light pulses.

In certain embodiments, the plurality of convertible residues comprise two or more types of convertible residues, wherein a first type of convertible residues are activatable by light of a first wavelength and a second type of convertible residues are activatable by light of a second wavelength.

In certain embodiments, the iterative spacing among the plurality of the convertible residues conforms to a resolution of the data writing device for selectively converting the convertible residues.

In certain embodiments, the selectively converting step does not require specific positioning of the writable polymer.

In certain embodiments, the conversion of the convertible residues into the second fluorescent state is non-uniform on the data encoded polymer.

In certain embodiments, the conversion of the convertible residues into the second fluorescent state is not limited to certain positions on the data encoded polymer.

In certain embodiments, the method further comprises stretching or combing the writable polymer (e.g., a writable DNA) on a solid support.

In certain embodiments, the method further comprises visualizing locations of the convertible residues using a dye.

In certain embodiments, the method further comprises locally illuminating the writable polymer.

In certain embodiments, the locally illuminating uses Stimulated Emission Depletion (STED) laser methods.

In certain embodiments, the method further comprises joining two or more data fields from two or more writable polymers end-to-end, resulting in a joined polymer comprising two or more data fields.

In certain embodiments, the method further comprises controlling the passage rate of the writable polymer through the nanopore of the writing device.

In certain embodiments, a plurality of writable polymers pass through the data writing device to write the same data (e.g., generating data redundancy).

In yet another aspect, further provided herein are methods for reading data from a polymer encoded with data, comprising:
providing the polymer encoded with data comprising a plurality of convertible residues iteratively spaced along and covalently linked to the backbone of the polymer, wherein each convertible residue of the plurality iteratively spaced along and covalently linked via the backbone of the polymer, wherein a first subset of the plurality of convertible residues are in a first fluorescent state and a second subset of the convertible residues are in a second fluorescent state, the first fluorescent state and the second fluorescent state being different; and
passing the writable polymer encoded with data through a data reading unit to read the encoded data on the polymer encoded with data.

In certain embodiments, the writable polymer is a writable nucleic acid polymer and the plurality of convertible residues are convertible nucleobases.

In certain embodiments, the convertible residues in the first fluorescent state can be converted into the second fluorescent state by light.

In certain embodiments, the data reading unit comprises a nanopore.

In certain embodiments, the data reading unit comprises a fluorescence scanner, fluorescence plate reader, gel imager, fluorescence spectrometer, fluorescence microscope, or flow cytometer.

In certain embodiments, the data reading unit is a sequencing device.

In certain embodiments, the method further comprises measuring current flow of electrolytes during passage of the writable polymer.

In certain embodiments, the method further comprises determining whether each of the plurality of convertible residues is in the first fluorescent state or the second fluorescent state based on the measured current flow of electrolytes during passage of the writable polymer.

In certain embodiments, the encoded data on the polymer encoded with data is read in sequence.

In certain embodiments, the encoded data on the polymer encoded with data is read in sequence from one end of the polymer to the other end of the polymer.

In certain embodiments, the method further comprises re-passing the polymer encoded with data through the data reading device to re-read the encoded data on the polymer encoded with data.

In certain embodiments, the method further comprises validating and correcting the encoded data on the polymer encoded with data by comparing the encoded data on multiple copies of the polymer encoded with data.

In yet another aspect, further provided herein are methods for reading or decoding data from a polymer encoded with data, the method comprising:
providing a plurality of redundant copies of the polymer encoded with data comprising:
   a plurality of converted residues, wherein each converted residue comprises a first fluorophore, wherein the first converted residue has been converted from a first fluorescent state into a second fluorescent state by light, the first fluorescent state and the second fluorescent state being different; and
   a plurality of convertible residues, wherein each convertible residue comprising a second fluorophore, and wherein the convertible residue is provided in a first fluorescent state and is capable of being converted from the first fluorescent state into a second fluorescent state light, the first fluorescent state and the second fluorescent state being different;
   wherein the converted residues and convertible residues are linked via the polymer backbone; and
sequencing each redundant copy of the plurality redundant copies of the polymer.

In certain embodiments, the method further comprises detecting the plurality of converted nucleobases and the plurality of convertible residues; and decoding the data based on the detected plurality of converted residues.

In certain embodiments, the plurality of converted residues in the first fluorescent state and the second fluorescent state are readable by polymerase.

In certain embodiments, the plurality of convertible residues in the first fluorescent state and the second fluorescent state are readable by polymerase.

In certain embodiments, the plurality of converted residues and the plurality of convertible residues are detected based on the sequencing result of the redundant copies of the nucleic acid polymer encoded with data.

In yet another aspect, further provided herein are methods for making a writable polymer for encoding data, the method comprising:
providing a polymer in a solution; wherein the polymer comprises iteratively spaced reactive moieties that are covalently linked to the backbone of the polymer;
adding sets of chemically modifiable structures to the solution, wherein each set of chemically modifiable structures is functionalized with a chemical group capable of reacting with the reactive moieties of the polymer thereby a writable polymer with iteratively spaced sets of chemically modifiable structures is yielded, and
wherein the chemically modifiable structures having a first state (e.g., a first fluorescent state) and are capable of being converted into a second state (e.g., a second fluorescent state), the first state and the second state being different; and
wherein the chemically modifiable structures are covalently linked to the polymer in the first state and the second state.

In yet another aspect, further provided herein are methods for making a writable nucleic acid polymer, the method comprising:
providing a single-stranded nucleic acid polymer in a first solution;
adding to the first solution a mixture comprising (i) a primer sequence complementary to the single-stranded nucleic acid polymer, (ii) a set of deoxynucleotides tri-phosphates, and (iii) a nucleic acid polymerase, wherein the set of deoxynucleotides tri-phosphates comprises modified deoxynucleotides tri-phosphates that have reactive moieties;
incubating the mixture in the first solution to yield a complement strand of the single-stranded nucleic acid polymer comprising modified nucleobases having the reactive moieties;
incubating the complement strand with sets of chemically modifiable structures in a second solution, wherein each set of chemically modifiable structures are capable of reacting with the reactive moieties of the modified nucleobases incorporated into the complement strand such that a writable nucleic polymer with iteratively spaced sets of chemically modifiable structures is yielded, and
wherein the chemically modifiable structures having a first state (e.g., a first fluorescent state) and are capable of being converted into a second state (e.g., a second fluorescent state), the first state and the second state being different; and
wherein the chemically modifiable structures are covalently linked to the polymer in the first state and the second state.

In yet another aspect, further provided herein are methods for making a writable organic polymer, the method comprising:
providing an organic polymer in a solution, wherein the organic polymer comprises iteratively spaced reactive moieties that are covalently linked to the backbone of the polymer;
adding sets of chemically modifiable structures to the solution, wherein each set of chemically modifiable structures are capable of reacting with the reactive moieties on the organic polymer such that a writable organic polymer with iteratively spaced sets of chemically modifiable structures is yielded, and
wherein the chemically modifiable structures having a first state (e.g., a first fluorescent state) and are capable of being converted into a second state (e.g., a second fluorescent state), the first state and the second state being different; and
wherein the chemically modifiable structures are covalently linked to the polymer in the first state and the second state.

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIGS. 1A and 1B provide schematics for a writable polymer in accordance with various embodiments.

FIGS. 2A to 2E provide molecular structure diagrams of various caged fluorophores for use in a writable polymer in accordance with various embodiments.

FIGS. 3A and 3B provide molecular structure diagrams of various releasable quenchers for use in a writable polymer in accordance with various embodiments.

FIG. 4 provides molecular structure diagrams of an exemplary photoconvertible fluorophore for use in a writable polymer in accordance with various embodiments.

FIG. 5A illustrates an example of a writable two-fluorophore bit utilizing two distinct caged fluorophores. FIG. 5B provides an exemplary modifiable fluorophore bit utilizing a quencher and caged fluorophore in accordance with various embodiments.

FIGS. 6A to 6C provide exemplary organic polymers incorporating modifiable fluorophores in accordance with various embodiments.

FIG. 7 provides a schematic of installing sets of chemically modifiable structures to a polymer to generate a writable polymer in accordance with various embodiments.

FIGS. 8A to 8C provide molecular structure diagrams of exemplary sets of chemically modifiable structures for use in a writable polymer in accordance with various embodiments.

FIG. 9 provides diagrams of exemplary reactions to install chemically modifiable structures onto nucleic acid polymers in accordance with various embodiments.

FIG. 10 provides examples of functional groups that can be utilized to install chemically modifiable structures onto polymers in accordance with various embodiments.

FIG. 11 provides a schematic of an exemplary method to install functional groups onto nucleic acid polymers via polymerase extension in accordance with various embodiments.

FIG. 12 provides a schematic of an exemplary method to install stable and alterable secondary conformations into a nucleic acid polymer in accordance with various embodiments.

FIG. 13 provides molecular structure diagrams of exemplary convertible residues for incorporating into a nucleic acid polymer for installing stable and alterable secondary conformations in accordance with various embodiments.

FIG. 14 shows an example of labeled DNA electrophoresis results where Lane 1 contains Cy5-labeled DNA and Lane M contains Safegreen 1 kb DNA ladder.

FIGS. 15A-15C provide DNA imaging results of Cy-5-labelled DNAs before and after bleaching. FIG. 15A shows an image of Cy5 labeled DNA before bleaching and FIG. 15B shows an image of Cy5 labeled DNA after localized photobleaching. FIG. 15C shows a plot displaying two cross section profiles: before bleaching (1501) and after bleaching (1502).

### DETAILED DESCRIPTION

Provided herein are compositions of data-encodable polymers (e.g., nucleic acid polymers), and methods and systems thereof, for data encoding/decoding (writing/reading) and data storage. Also provided herein are method of making the polymers (e.g., nucleic acid polymers) described herein.

Described herein are compositions and systems of data storage utilizing polymers, methods of use and methods of synthesis, in accordance with various embodiments. In several embodiments, a system of data storage comprises writable polymers having one or more convertible residues. In some embodiments, the convertible residues comprise convertible nucleotides. In several embodiments, a system of data storage comprises writable (i.e., data-encodable) polymers having one or more residues that are convertible.

Accordingly, a writable nucleic acid polymer is akin to a blank tape that is encodable, wherein the writable nucleic acid polymer is encoded by converting one or more its nucleobases. Conversion of convertible residues can be thought of as a binary code, where each convertible residue is akin to a “bit,” unconverted convertible residues are akin to a “0,” and convertible residues that have been converted are akin to a “1.” It should be understood, however, that a binary code is not the only possibility, and codes can be written in ternary, quaternary, or other numeral system code, which can be done utilizing multiple types of convertible residues or performing multiple writings to further alter the state of a convertible residue. In some embodiments, the conversion of a convertible residue is stable, or permanent, which allows for long-term archiving. In some embodiments, the combination of two convertible residues comprises a “bit”.

In some embodiments, a first fluorescent state comprises a blank state (e.g., unwritten state), a “0” state, or a “1” state. In some embodiments, a second fluorescent state comprises a blank state (e.g. unwritten state), a “0” state, or a “1” state. In some embodiments, a third fluorescent state comprises a blank state (e.g. unwritten state), a “0” state, or a “1” state. In some embodiments, a first fluorescent state comprising a blank state may be converted to a second fluorescent state comprising a “1” state. In some embodiments, a first fluorescent state comprising a “1” state may be converted to a second fluorescent state comprising a “0” state.

In some embodiments, the conversion of the convertible residue from a first state to a second state is executed by a writing device. In some embodiments, the writing device comprises a light impinging module (e.g., light source). In some embodiments, the state (e.g., first state, second state, third state, etc. . . . ) is detected by a reading device or unit. In some embodiments, the reading unit comprises the writing device. In some embodiments, the reading unit comprises a (fluorescence) detection device. In some embodiments, the reading unit comprises an analysis module.

In some embodiments, the conversion of the convertible residues comprises any detectable change of fluorescence state, including photoactivation of a fluorophore, inactivation of a fluorophore (e.g., by light), release of a fluorophore, uncaging of a caged fluorophore, quenching of a fluorophore by a quencher (e.g., a quencher release from a convertible residue), and photobleaching of a fluorophore.

In some embodiments, convertible residues may comprise a fluorophore. In some embodiments, the fluorophore may comprise a modifiable fluorophore. In some embodiments, the convertible residue may comprise a leaving group. In some embodiments, the leaving group may be a quencher or a cage (e.g., photo-removeable group or photo-cleavable group). In some embodiments, the fluorophore comprises the leaving group. In some embodiments, the leaving group of the fluorophore may be the cage. In some embodiments, the fluorophore may be a caged fluorophore (e.g., the fluorophore comprising the cage). In some embodiments, the cage may be the leaving group of the modifiable fluorophore. In some embodiments, the convertible residue may be a convertible fluorophore.

In some embodiments, the convertible residue comprises the leaving group, wherein the leaving group may be a quencher.

In some embodiments, the convertible residue comprises a modifiable fluorophore that can be activated by light. In some embodiments, the modifiable fluorophore can be activated by light in the presence of an additive (e.g., a phosphine). In some embodiments, the convertible residue comprises a modifiable fluorophore that can be inactivated by light.

In some embodiments, the modifiable fluorophore can be inactivated by light in the presence of an additive (e.g., a phosphine).

In some embodiments, the convertible residue comprises a releasable fluorophore that is capable of being released from the polymer by light.

In some embodiments, the convertible residue comprises a photobleachable fluorophore that is capable of being photobleached by light.

Described herein are various compositions, systems, methods of making and methods of use, for a (writable) polymer for encoding data, comprising: a plurality of convertible residues iteratively spaced along and covalently linked to the backbone of the polymer, wherein each convertible residue of the plurality of convertible residues has a first fluorescent state, and is capable of being converted from the first fluorescent state into a second fluorescent state, the first fluorescent state and the second fluorescent state being different; wherein each of all or a subset of the plurality of convertible residues comprises a fluorophore, and wherein the plurality of convertible residues are covalently linked to the polymer in the first fluorescent state and in the second fluorescent state.

In some embodiments, wherein each of the convertible residues of the plurality of convertible residues are iteratively spaced along the backbone of the polymer, iteratively spaced can be referred to as approximately regularly spaced.

In some embodiments, a convertible residue (e.g., a residue comprising a modifiable fluorophore or a residue comprising a releasable quencher) is referred to as a writable “bit,” and a converted residue (e.g., a converted fluorophore with altered emission) is referred to as a written “bit.”

In some embodiment, the terms “writable” and “data-encodable” are used herein interchangeably. In some embodiment, the terms “writing” and “data encoding” are used herein interchangeably.

In some embodiments, the terms “leaving group” and “removable group” are used herein interchangeably. In some embodiments, when referring to convertible residues, the terms “pair” and “duad” are used herein interchangeably. “Duad,” used herein refer to a pair of different convertible residues (e.g., writable bits) that are located close enough relative to one another in the polymers described herein (e.g., nucleic acid polymers) such that both are exposed to a single writing action or event (e.g., the same pulse of light or the same voltage pulse). Thus, the convertible residues that comprise the duad may be closer than the resolution of the writing action or event.

In several embodiments, the one or more convertible residues comprise one or more modifiable fluorophores. Accordingly, a writable polymer is akin to a blank tape that is encodable, wherein the writable polymer is encoded by turning on/off or converting a fluorophore, which can be done by any method in which a fluorophore can be modified. In various embodiments, fluorophores are modified by uncaging, unquenching, and/or photoconverting, depending on the modification mechanism utilized. Fluorophore modification can be thought of as a binary code, where a modifiable fluorophore is akin to a “bit;” one state of a fluorophore is akin to a “0,” and a second state of a fluorophore akin to a “1”. For instance, in one example, a caged fluorophore can be akin to “0” and an uncaged fluorophore can be akin to a “1”. It should be understood, however, that a binary code is not the only possibility, and codes can be written in ternary, quaternary, or other numeral system code, which can be done utilizing multiple types of fluorophores or performing multiple writings/modifications to further alter the state of a fluorophore. The modification of a fluorophore can be stable, or permanent, which allows for long-term archiving, especially if kept in a dark storage location. In some embodiments, the combination of two uniquely identifiable fluorophores comprises a “bit”. For instance, a caged fluorophore and a quenched fluorophore can be utilized to be a single bit, wherein an uncaged fluorophore having a first fluorescent emission intensity or wavelength can be akin to a “0” and an unquenched fluorophore having a second fluorescent emission intensity or wavelength can be akin to a “1”.

In some embodiments, the nucleic acid polymer is a single-stranded nucleic acid polymer or a double-stranded nucleic acid polymer. In some embodiments, the nucleic acid polymer is a single-stranded nucleic acid polymer. In some embodiments, the nucleic acid polymer is a double-stranded nucleic acid polymer.

Many embodiments are directed towards compositions of writable polymers. Any appropriate iteratively charged polymer can be utilized, including (but not limited to) biological polymers, organic polymers, and inorganic polymers. Biological polymers (and their analogues) include (but are not limited to) DNA, RNA, phosphorothiate DNA, glycerol nucleic acids (GNA), threose nucleic acids (TNA), 2′-fluoro-DNA 2′-O-methyl RNA, locked nucleic acids (LNA), peptide chains, and peptoid chains. A nucleic acid polymer may be single stranded or double stranded. Further, a nucleic acid polymer may utilize any enantiomer (e.g., R-DNA, L-DNA). Similarly, a peptide polymer can be any enantiomer. In some embodiments, a polymer is iteratively charged by having a charged backbone. In some embodiments, a polymer is iteratively charged by incorporating monomers having a charged constituent. In various embodiments, iterative charge of a polymer is provided by negatively charged phosphate groups, negatively charged sulfate groups, negatively charged carboxylate groups, or positively charged ammonium groups.

In some embodiments, any appropriate nucleic acid polymer can be utilized, including (but not limited to) DNA, RNA, phosphorothioate DNA, glycerol nucleic acids (GNA), 2′-OMe-RNA, threose nucleic acids (TNA), locked nucleic acids (LNA), and combinations thereof.

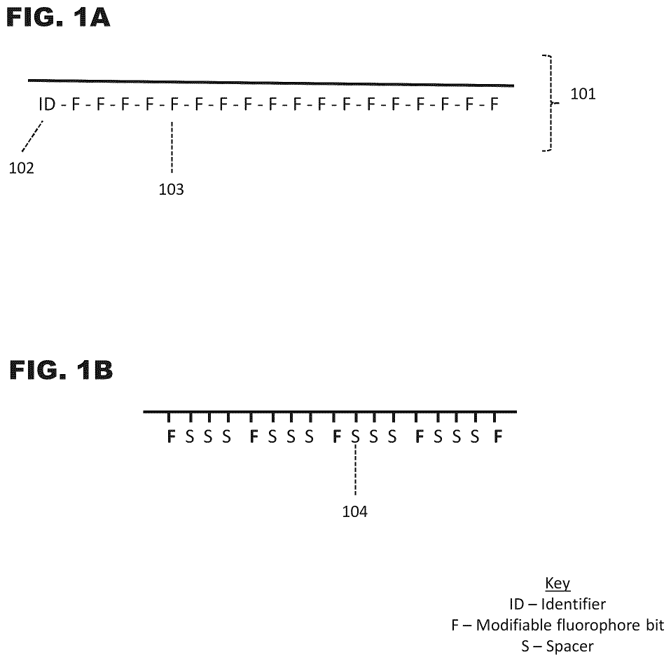

In several embodiments, a writable nucleic acid polymer comprises a plurality of modifiable fluorophores that are linked by a polymer backbone. In certain embodiments, modifiable fluorophore bits are spaced apart to provide spatial resolution such that each fluorophore bit can be independently and selectively modified in accordance with data encoding. In some embodiments, spacer residues linked via the polymer backbone are utilized to provide spaces between the modifiable fluorophore bits as depicted in FIG. 1B. In various embodiments, a writable nucleic acid polymer can further include delimiters and/or data tags for labeling or locating the data as depicted in FIG. 1A. In some embodiments, the delimiter may comprise a fluorophore.

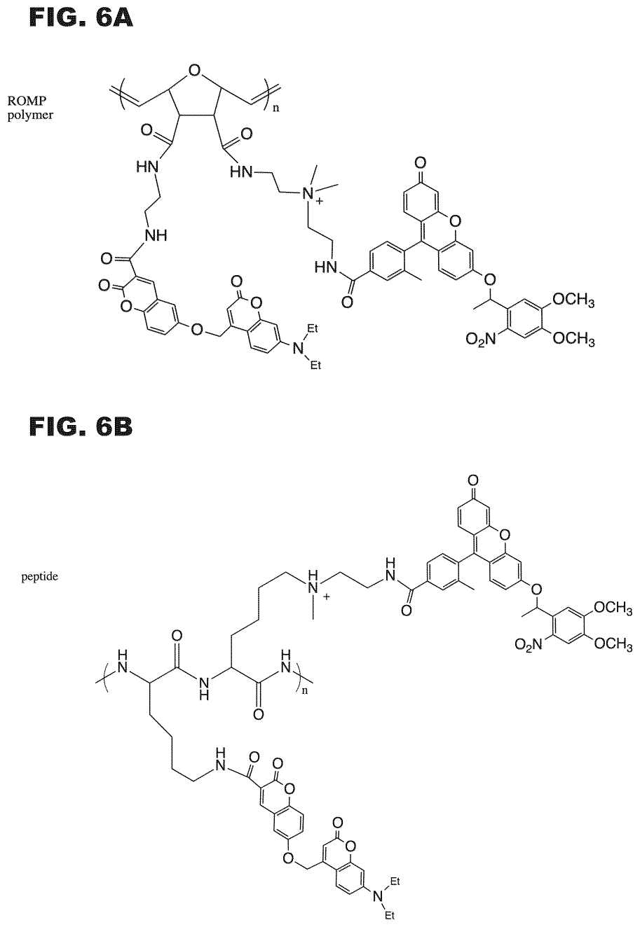

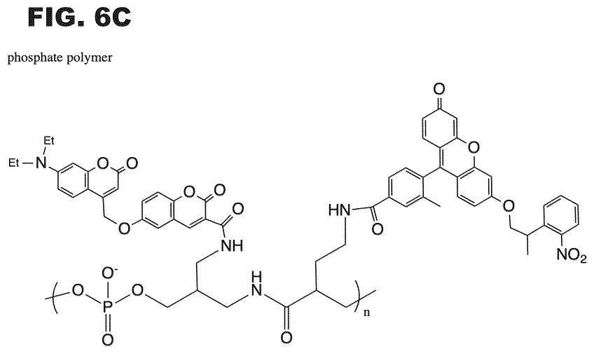

In some embodiments, modifiable fluorophores are linked by a ROMP polymer backbone as depicted in FIG. 6A. In some embodiments, modifiable fluorophores are linked by a peptide polymer backbone as depicted in FIG. 6B. In some embodiments, modifiable fluorophores are linked by a phosphate polymer backbone as depicted in FIG. 6C.

In several embodiments, a writing procedure is utilized to encode a writable polymer with data. Data encoding can be performed by selectively modifying fluorophore bits of a polymer such that the written polymer contains a sequence of modified fluorophores, akin to a binary code of “zeros” and “ones”. Any appropriate mechanism to modify a fluorophore (e.g., turn on/off; modulate fluorescence wavelength) can be utilized. In accordance with various embodiments, a fluorophore is modifiable via light.

In some embodiments, the writing procedure occurs in sequence along the writable polymer. In some embodiments, the writing procedure occurs starting from one end of the polymer and proceeds to the other end of the polymer in sequence.

In accordance with many embodiments, data written polymers are stored in the dark free of photobleaching light. In some embodiments, data written polymers are stored in environments that exclude air or oxygen, which may enhance stability. Stabilizers such as (for example) alcohol, antioxidants, chelating agents and biological inhibitors (e.g., nuclease inhibitors or protease inhibitors), may be included with the stored polymer. To read the data on written nucleic acid polymers, any appropriate nanopore or single-mode waveguide capable of detecting individualized fluorophores can be utilized, such as Pacific Bioscience's Single Molecule, Real-Time (SMRT) sequencing platform (Menlo Park, CA). Alternatively, a nanopore capable of analyzing structural differences in monomers can be utilized, such as Oxford Nanopore Technologies PromethION, MinION, and GridION sequencing platforms (Oxford, UK). Also, a nanopore device can be fabricated or manufactured for reading the data. The nanopore can be comprised of solid-state materials, or can contain one or more proteins or other biologically produced molecules.

The present disclosure overcomes many of the limitations associated with traditional nucleic acid and polymer data storage by separating the synthesis and data encoding into distinct steps. Traditionally, polymer data storage has been focused on DNA. And while several embodiments utilize biological polymers for data storage, many embodiments of the disclosure, however, utilize nonbiological polymers. The disclosure provides strategies for producing long strands of writable polymers having modifiable fluorophores, wherein the production of the polymer does not encode data, but rather a polymer is produced having the capacity for data being written. And thus, writable polymers can be produced in bulk in advance of data encoding. The disclosure further provides molecular strategies for the design of modifiable fluorophores that act as “bits” of data, which can be switched from a first state into a second state, thus defining “0” and “1” in binary code, or defining the change from a “blank” state to a “0” bit or “blank” to “1” bit. The disclosure further provides methods for writing data into these polymers at the single molecule level, thus consuming negligible amounts of material. Data writing may be achieved physically, utilizing (for example) light pulses. Finally, because the written polymers are long, they potentially encode a high amount of data per molecule and can be efficiently and rapidly read via high-throughput nanopore techniques. Advances in nanopore technology have lowered the cost and increased the speed of analysis, allowing data in polymers to be read efficiently. Newer nanopore technologies enable the reading of sequence from single molecules of polymers in seconds to minutes (see N Kono and K. Arakawa, Dev Growth Differ. 2019; 61:316-326; and Q Chen and Z. Liu, Sensors (Basel). 2019; 19:1886; the disclosures of which are each incorporated herein by reference), and can read sequences of strands tens of thousands of monomers in length or more. The compositions, systems, and methods described herein greatly increase the speed and density of polymeric data encoding while lowering cost.

### Writable Polymers for Encoding Data

In one aspect, provided herein are polymers for encoding data.

In some embodiments, the polymers for encoding data comprise a plurality of convertible residues iteratively spaced along and covalently linked to the backbone of the polymer, wherein each convertible residue of the plurality of convertible residues has a first fluorescent state, and is capable of being converted from the first fluorescent state into a second fluorescent state, the first fluorescent state and the second fluorescent state being different; wherein each of all or a subset of the plurality of convertible residues comprises a fluorophore, and wherein the plurality of convertible residues are covalently linked to the polymer in the first fluorescent state and in the second fluorescent state. In some embodiments, the first fluorescent state and the fluorescent second state are different (e.g., the convertible residues have different structures when in the first and the second state). In some embodiments, the plurality of convertible residues in the first fluorescent state and in the second fluorescent state are readable by a fluorescence detection device. In some embodiments, each of the convertible residues of the plurality of convertible residues are repeatedly spaced along the backbone of the polymer.

In some embodiments, the fluorescence detection device is a fluorescence plate reader, gel imager, fluorescence spectrometer, fluorescence microscope, or a flow cytometer.

In some embodiments, the polymers described herein are nucleic acid polymers and the plurality of convertible residues comprise convertible nucleobases.

In some embodiments, the convertible residues are iteratively spaced apart to provide spatial resolution such that each residue can be independently converted. In some embodiments, any appropriate spacer (e.g., non-writable, i.e., unreactive to the data writing mechanism) are between the convertible residues. In some embodiments, residues linked by the polymer backbone can be utilized as spacers. In some embodiments, the spacers spaced between the convertible residues in accordance with the spatial resolution of the writing mechanism and/or writing device. In some embodiments, spacers are residues, which may be unreactive to the writing mechanism. In some embodiments, these spacers are unmodified DNA nucleotides. In various embodiments, the polymer further comprises delimiters and/or data tags for labeling the data.

In some embodiments, the polymers described herein (e.g., nucleic acid polymers) further comprise a plurality of spacer residues linked via the backbone of the polymer. Each of the plurality of convertible residues are separated by the one or more spacer residues of the plurality of spacer residues. In some embodiments, wherein the amount of (e.g., distance of) spacing among the plurality of convertible residues conforms to a resolution of a writing mechanism for encoding data on the polymer. In some embodiments, the spacing among two convertible residues is equal to or greater than a resolution of a data encoding mechanism for encoding data into the polymer. In some embodiments, the resolution of the writing mechanism is at least 1 nm. In some embodiments, the plurality of spacer residues do not interfere with reading of the convertible residues. In some embodiments, the plurality of spacer residues in the polymer are the same spacer residues. In some embodiments, the plurality of spacer residues comprises two or more different spacer residues (e.g., different nucleotides such as different occurring nucleotides).

In some embodiments, two or more different convertible residues fall within the resolution of the writing mechanism. In some embodiments, the two or more different convertible residues falling within the resolution of the writing mechanism respond to different wavelengths of light, occurring within two simultaneous light pulses.

In some embodiments, the polymers described herein are blank tapes. In some embodiments, the polymers described herein are blank tapes of DNA. Blank tape used herein refers to a writable nucleic acid polymer that comprises convertible residues iteratively spaced along the writable nucleic acid polymer, such that conversion of convertible residue from a first state into a second state results in encoding of data. The blank tape itself contains no data, but is capable of being encoded with data by use of an appropriate writing system (e.g., by light) via converting the convertible residue.

In some embodiments, the blank tape is writable over its entire length. In some embodiments, each convertible residue in the blank tape is independently and individually writable.

In some embodiments, the polymers described herein (e.g., nucleic acid polymers) consist essentially of spacer residues.

In some embodiments, the polymers described herein (e.g., nucleic acid polymers) comprise no delimiter or data tag.

In some embodiments, the polymers described herein (e.g., nucleic acid polymers) consist of spacer residues and convertible residues.

In some embodiments, each of the plurality of convertible residues are separated by 2, 3, 4, 5, 6, 7, 8, 9, 10, 20, 30, 40, or 50 spacer residues. In some embodiments, each of the plurality of convertible residue are separated by 6 spacer residues. In some embodiments, the plurality of spacer residues comprise naturally occurring nucleobases, non-natural nucleobases, tetrahydrofuran abasic residues, or ethylene glycol residues. In some embodiments, the plurality of spacer residues comprise naturally occurring residues.

In some embodiments, the polymers described herein (e.g., nucleic acid polymers) further comprise one or more delimiters linked to the backbone of the polymer. In some embodiments, each of the one or more delimiters comprises one or more naturally occurring nucleobases or non-naturally nucleobases. In some embodiments, the one or more delimiters comprise naturally occurring nucleobases. In some embodiments, the one or more delimiters separate two or more adjacent data fields within the polymer. In some embodiments, the delimiter may be a fluorophore. In some embodiments, the delimiter is a fluorophore to serve as a detectable marker of distance or position.

In some embodiments, the polymers described herein (e.g., nucleic acid polymers) further comprise one or more data tags. In some embodiments, the one or more data tags comprise one or more naturally occurring residues (e.g., a naturally occurring residue) or non-natural residues (e.g., a non-natural residue). In some embodiments, the polymer is a nucleic acid polymer and the one or more data tags are present at the 5′ or 3′ end of the nucleic acid polymer. In some embodiments, the one or more data tags are incorporated to the nucleic acid polymer during the nucleic acid polymer is synthesized, during the plurality of convertible residues are converted to the second state, or via ligation after the plurality of convertible residues are converted to the second state.

In some embodiments, the polymer can have any number or length of monomeric units, for example, from as short as 10 monomeric units to longer than 100,000 monomeric units. In various embodiments, the polymer has greater than 500 monomeric units, greater than 1,000 monomeric units, greater than 5000 monomeric units, greater than 10,000 monomeric units, greater than 50,000 monomeric units, or greater than 100,000 monomeric units.

In some embodiments, the nucleic acid polymer comprises greater than 10 convertible residues. In some embodiments, the nucleic acid polymer comprises greater than 100 convertible residues. In some embodiments, the nucleic acid polymer comprises greater than 500 convertible residues. In some preferred embodiments, the nucleic acid polymer comprises greater than 1,000 convertible residues. In some embodiments, the nucleic acid polymer comprises greater than 10,000 convertible residues. In some embodiments, the nucleic acid polymer comprises greater than 100,000 convertible residues.

In some embodiments, the ratio of the total number of monomeric units (e.g., nucleotides) to the convertible residues in the polymer (e.g., nucleic acid polymer) is between 2 to 500. In some embodiments, the ratio of the total number of monomeric units (e.g., nucleotides) to the convertible residues in the polymer (e.g., nucleic acid polymer) is between 2 to 200. In some embodiments, the ratio of the total number of monomeric units (e.g., nucleotides) to the convertible residues in the polymer (e.g., nucleic acid polymer) is between 2 to 100. In some embodiments, the ratio of the total number of monomeric units (e.g., nucleotides) to the convertible residues in the polymer (e.g., nucleic acid polymer) is between 2 to 10. In some embodiments, the ratio of the total number of monomeric units (e.g., nucleotides) to the convertible residues in the polymer (e.g., nucleic acid polymer) is between 10 to 50.

In some embodiments, the ratio of the total number of monomeric units (e.g., nucleotides) to the convertible residues in the polymer (e.g., nucleic acid polymer) is between 10 to 100. In some embodiments, the ratio of the total number of monomeric units (e.g., nucleotides) to the convertible residues (e.g., convertible in the polymer (e.g., nucleic acid polymer) is between 20 to 100. In some embodiments, the ratio of the total number of monomeric units (e.g., nucleotides) to the convertible residues in the polymer (e.g., nucleic acid polymer) is between 20 to 50. In some embodiments, the ratio of the total number of monomeric units (e.g., nucleotides) to the convertible residues in the polymer (e.g., nucleic acid polymer) is greater than 100.

Compounds in accordance with embodiments of the disclosure are based on iteratively charged polymers having a plurality of modifiable fluorophores, which are akin to writable data bits. Each “fluorophore bit” can exist in two or more states, a first state akin to a “0”, and at least a second state akin to a “1”, and in some embodiments, additional states. Alternatively, a first state can encode a “blank” (unwritten) state, a second state can encode a “0” bit, and a third state can encode a “1” bit. In several embodiments, writable polymers are synthesized with a plurality of modifiable fluorophore bits in an unwritten state that are capable of being modified, akin to a “blank tape”. In some embodiments, a data bit consists of a single modifiable fluorophore. In some embodiments, a single modifiable fluorophore and a constitutive fluorophore are employed as a pair for encoding a single bit; when only the constitutive fluorophore is fluorescent the bit encodes a “0”; whereas when the constitutive fluorophore is fluorescent and the modifiable fluorophore is modified to be fluorescent, the data bit encodes a “1”. In some embodiments, two different modifiable fluorophores are employed as a pair for encoding a single data bit; modifying of a first modifiable fluorophore encodes a “0” while modifying of the second fluorophore encodes a “1”. And in some embodiments, two different modifiable fluorophores are employed as a pair for encoding a single data bit; modifying of a first modifiable fluorophore encodes a “0” while modifying of the first and second fluorophore encodes a “1”.

Writable polymers can be created having long lengths (e.g., 5 to 100,000 monomers, or more) and can be produced in bulk, prior to data writing.

In several embodiments, a writable polymer comprises a plurality of monomers with modifiable fluorophores that are linked by the polymer backbone. In certain embodiments, modifiable fluorophore bits are iteratively spaced apart to provide spatial resolution such that each fluorophore bit can be independently modified. The spatial resolution depends, at least in part, on the writing mechanism. For instance, if an optical light source and device with 1 nm of resolution is used to modify fluorophores, then each fluorophore bit can be separated by at least 1 nm. Any appropriate spacer between the fluorophore bits can be utilized. In some embodiments, residues linked by the polymer backbone can be utilized as spacers. In some embodiments in which nucleic acids are utilized as the polymer, spacers are nucleobases without an attached fluorophore. In various embodiments, a writable polymer can further include delimiters and/or data tags for labeling, marking, or separating the data, each of which can be provided by a particular sequence of residues.

FIG. 1A illustrates an exemplary concept of a writable polymer 101 having a plurality of modifiable fluorophore bits (e.g., modifiable fluorophore or “F”) 103. The writable polymer comprises a number of repeating modifiable fluorophore bits that can undergo structural changes from one fluorescent state to a second fluorescent state, akin to a switch from a “0” state to a “1” state. Each of these modifiable fluorophore bits is akin to a “bit” for data encoding. It is understood that the definition of “1” and “0” is arbitrary, and simply meant to signify binary code. Prior to any data writing, modifiable fluorophore bits are initially provided in an initial, unwritten state. In some embodiments, the writable polymer comprises data fields that include a plurality of modifiable fluorophore bits and may also contain spacers 104 or delimiters. FIG. 1B provides an exemplary concept of a polymer sequence having a plurality of modifiable fluorophore bits separated by spacers. In some embodiments, a writable polymer includes one or more unique data tag sequences, denoting documentation such as type of data, date, or other information. A unique data tag may be added on to an end via a chemical or enzymatic attachment mechanism, or by further extending the polymer in a new block. See FIG. 1A for an example of a data tag (ID) 102 attached to the polymer.

In various embodiments, writable polymers can be any length, for example, from as short as 15 monomers to longer than 5000 monomers in most organic polymers, to longer than 100,000 monomers in nucleic acid polymers. In various embodiments, a writable polymer is greater than 100 monomers long, is greater than 200 monomers, is greater than 300 monomers, is greater than 400 monomers, is greater than 500 monomers, is greater than 1000 monomers, is greater than 5000 monomers, is greater than 10,000 monomers, is greater than 50,000 monomers, or is greater than 100,000 monomers. Maximum lengths are only limited by the stability of the selected polymer type, by the method used to make them, and by the method used to read the written data. Longer strands containing more bits have the advantage of containing more data per molecule.

In some embodiments, the plurality of convertible residues are capable of being incorporated into the nucleic acid polymer by one or more polymerase enzymes.

In some embodiments, the plurality of convertible residues are non-naturally occurring residues (e.g., non-naturally occurring nucleobase). In some embodiments, the plurality of convertible residues are modified naturally occurring residues (e.g., naturally occurring nucleobases) or derivatives of naturally occurring residues.

In some embodiments, each of the plurality of convertible residues comprises a chemically modifiable moiety (e.g., a fluorophore). In some embodiments, in each of the plurality of convertible residues the chemically modifiable moiety (e.g., a fluorophore) is directly attached to the convertible residue. In some embodiments, in each of the plurality of convertible residues the chemically modifiable moiety (e.g., a fluorophore) is attached to the convertible residues without a linker or a sidechain. In some embodiments, the plurality of convertible residues are covalently linked to the backbone of polymer.

In some embodiments, a linker comprises at least one atom. In some embodiments, linking of two moieties without a linker refers to linking without any intervening atoms.

In some embodiments, the convertible residues are linked to the backbone of the polymer (e.g., a nucleic acid polymer) in the same way that a nucleobase in a native nucleotide is linked to the backbone of the nucleic acid polymer (via the sugar in a nucleotide), without an intervening linker or as a sidechain.

In one embodiment, the chemically modifiable moiety is activatable by light, thereby converting from the first fluorescent state into the second fluorescent state. In some embodiments, the conversion from the first fluorescent state into the second fluorescent state occurs via an irreversible reaction. In some embodiments, the convertible residue has a caged fluorophore in the first fluorescent state and has an uncaged fluorophore after conversion into the second fluorescent state. In some embodiments, the convertible residue has a quencher-bearing fluorophore in the first fluorescent state and the quencher is released after conversion into the second fluorescent state. In some embodiments, the backbone of the polymer (e.g., phosphate and sugar in nucleic acid polymer) remain unchanged during the conversion from the first fluorescent state into the second fluorescent state. In some embodiments, the chemically modifiable moiety is activatable by light, voltage, enzymatic agent, chemical reagent, or a redox agent or redox electrode, thereby converting from the first state into the second state. In some embodiments, the chemically modifiable moiety comprises one or more photo-removable groups.

Several embodiments are directed to modifiable fluorophore bits, which can be incorporated into a writable polymer. A modifiable fluorophore bit, in accordance with various embodiments, is a one or more fluorophores that are capable of being structurally altered from a first fluorescent state (e.g., fluorescence off or low) into a second fluorescent state (e.g., fluorescence on or high) by controlled light reaction chemistry. In some embodiments, a modifiable fluorophore is a fluorophore that is caged, where the caged fluorophore is a first fluorescent state and release of the cage results in a second fluorescent state. In some embodiments, a modifiable fluorophore is a quenched fluorophore, where the quenched fluorophore is a first fluorescent state and release of the quencher results in a second, usually brighter, fluorescent state. In some embodiments, a modifiable fluorophore is a fluorophore capable of being converted from a first state having a particular fluorescent quality (e.g., a first emission or absorption wavelength) into a second state having a second distinguishable fluorescent quality (e.g., a second emission or absorption wavelength). In some embodiments, a modifiable fluorophore is paired with a constitutive fluorophore to provide a bit. A constitutive fluorophore is a fluorophore that provides consistent and unregulated fluorescence on its own. Constitutive fluorophores can be utilized as a positive control and/or provide a means to detect the location of bit when the bit includes a modifiable fluorophore that remains nonfluorescent (e.g., due to caging or quenching). A constitutive fluorophore may also be modified indirectly by the presence or absence of a quencher nearby, and modifying or releasing the quencher may thus indirectly cause the constitutive fluorophore to change its signal.

In some embodiments, the change of a (fluorescence) state, upon conversion of the convertible residue from the first (fluorescence) state to the second (fluorescence) state includes, but not limited to, a change in intensity, a change in absorption wavelength, a change in emission wavelength, a change in fluorescence lifetime or a combination thereof.

In some embodiments, converting a convertible residue may comprise releasing a cage from a caged fluorophore of the convertible residue. In some embodiments, converting a convertible residue may comprise releasing quencher of a second convertible residue in close proximity of a first convertible residue comprising a fluorophore. In some embodiments, converting a convertible residue may comprise photobleaching a fluorophore the convertible residue. In some embodiments, converting a convertible residue may comprise releasing a fluorophore of the convertible residue. In some embodiments, converting a convertible residue may comprise photoactivating the fluorophore of the convertible residue. In some embodiments, converting a convertible residue may comprise photo-inactivating the fluorophore of the convertible residue.

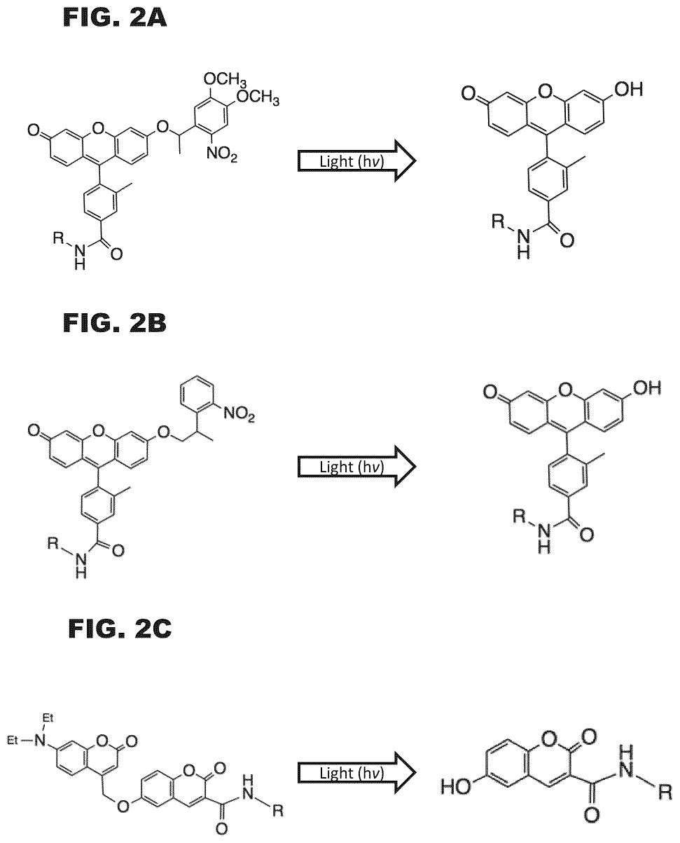

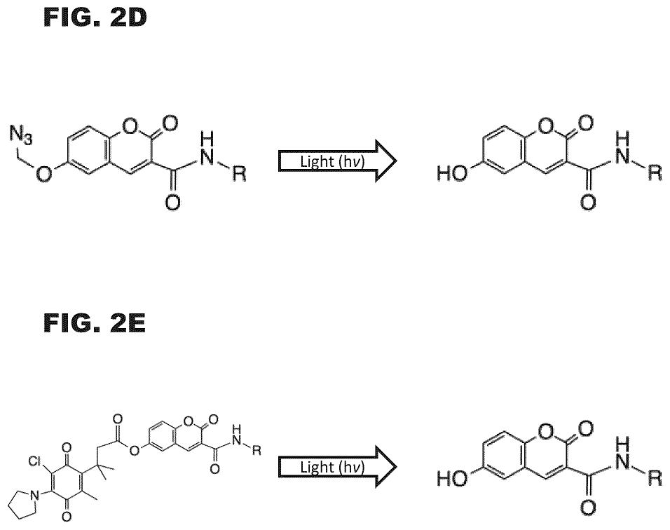

Provided in FIGS. 2A to 2E are examples of caged fluorophores in their caged and uncaged states, which can be utilized in association with monomers in a polymer linked by backbone. In several embodiments, caged fluorophores can be utilized in manner akin to “bits” of data, enabling conversion from a first structure state (e.g., caged) to a second structure state (e.g., uncaged), akin to “0” or “1” digital bit designations. In some embodiments, a constitutive fluorophore is utilized in conjunction with the caged fluorophore to help locate the detection of the caged fluorophore. Alternatively, a caged fluorophore can be utilized with a second modifiable fluorophore to provide a bit. The fluorescent state of the bit is detectable utilizing a nanopore device capable of detecting fluorescence of individual fluorophore molecules, or by a nanopore device that can detect the structural differences in the written bits.

In some embodiments, the plurality of modifiable fluorophores are caged fluorophores that are capable of being converted to uncaged fluorophores by light.

In some embodiments, each of the caged fluorophores comprises:

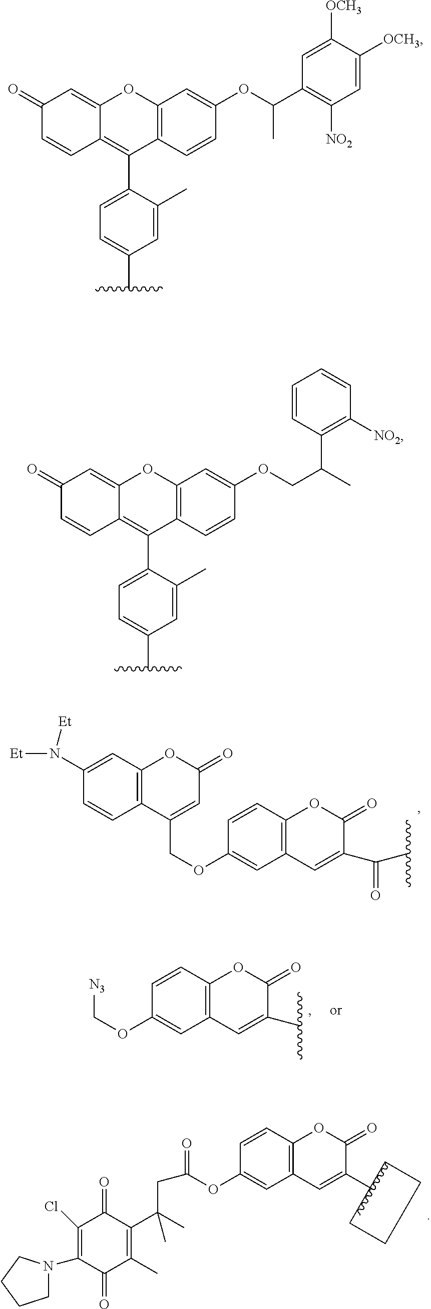 [SMILES: *c1ccc(-c2c3ccc(=O)cc-3oc3cc(OC(C)c4cc(OC)c(OC)cc4[N+](=O)[O-])ccc23)c(C)c1 ; *c1ccc(-c2c3ccc(=O)cc-3oc3cc(OCC(C)c4ccccc4[N+](=O)[O-])ccc23)c(C)c1 ; *C(=O)c1cc2cc(OCc3cc(=O)oc4cc(N(CC)CC)ccc34)ccc2oc1=O ; *c1cc2cc(OCN=[N+]=[N-])ccc2oc1=O ; *c1cc2cc(OC(=O)CC(C)(C)C3=C(C)C(=O)C(N4CCCC4)=C(Cl)C3=O)ccc2oc1=O]

Each of the caged fluorophores provided in FIGS. 2A to 2E are examples of cages that can released by localized pulses of light, resulting in a fluorophore capable of providing detectable fluorescence. Various photo removable groups can be incorporated into caged fluorophores (see, e.g., Y. Zhao, et al., J Am Chem Soc. 2004; 126:4653-63; the disclosure of which is incorporated herein by reference). As can be seen in the figures, each fluorophore has a caging constituent that is linked by a linker (e.g., ether group) that is cleavable with light energy. The fluorophores can be attached directly to the polymer backbone or to a residue attached to the backbone, as depicted in the figures by the R group. While a few examples are provided, it is understood that any appropriate photo-removable group and fluorophore may be used in accordance with the various embodiments. In some embodiments, a photo-removable group may be referred to as a photo-cleavable group or a cage.

In some embodiments, the one or more photo-removable or photo-cleavable groups are:

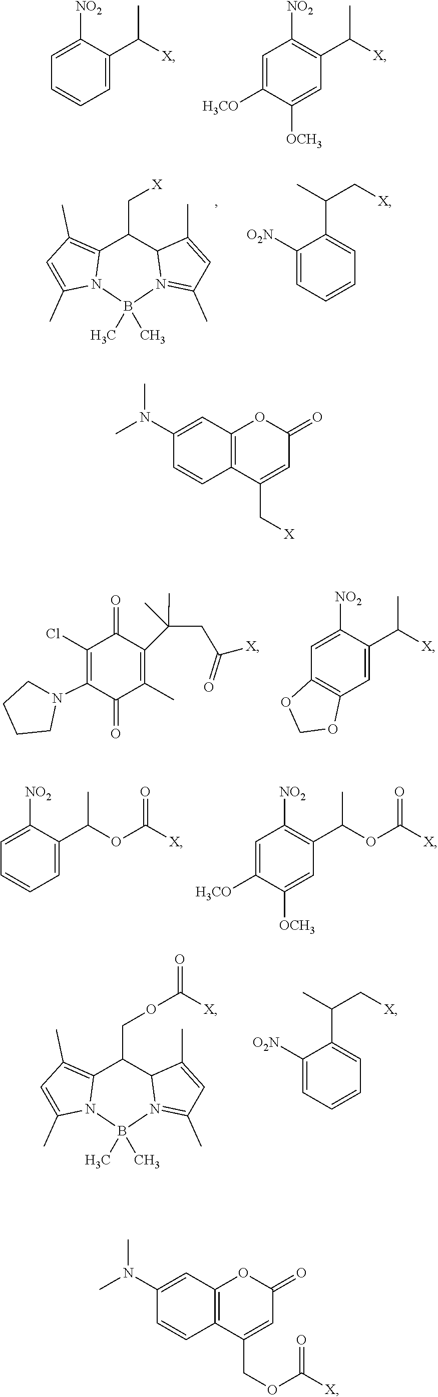

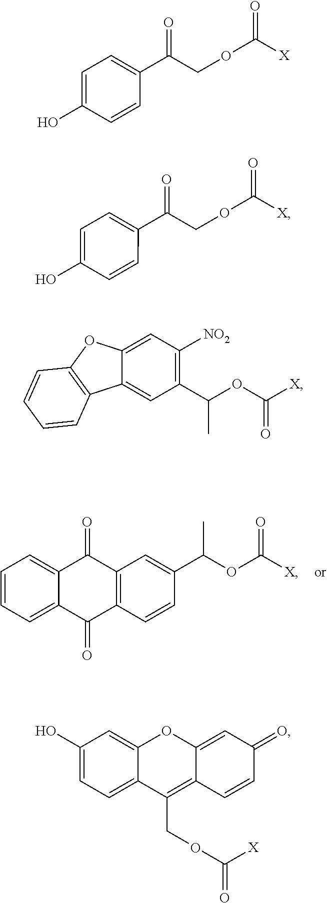

wherein X represents NR2, NHR, OR, or SR, and wherein R is the atom to which the photo-removable or photo-cleavable group is attached in the polymer.

In some embodiments, each of the plurality of convertible residues comprises a chemically modifiable moiety that is activatable by redox. In some embodiments, the chemically modifiable moiety is capable of being activated by localized oxidation. In some embodiments, the chemically modifiable moiety is capable of being activated by oxidation using electrodes.

In some embodiments, each of the plurality of convertible residues comprises a modifiable moiety (e.g., modifiable fluorophore, caged fluorophore, or a quencher) that is modifiable by light.

In some embodiments, a nucleotide comprising the convertible residue (e.g., convertible nucleobase) is selected from the group consisting of

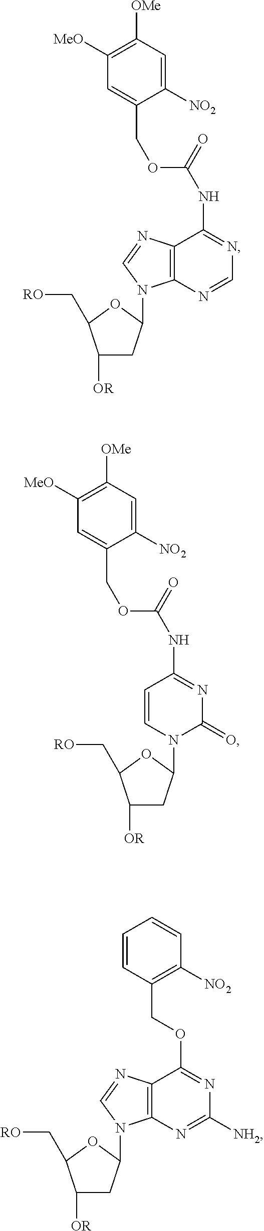

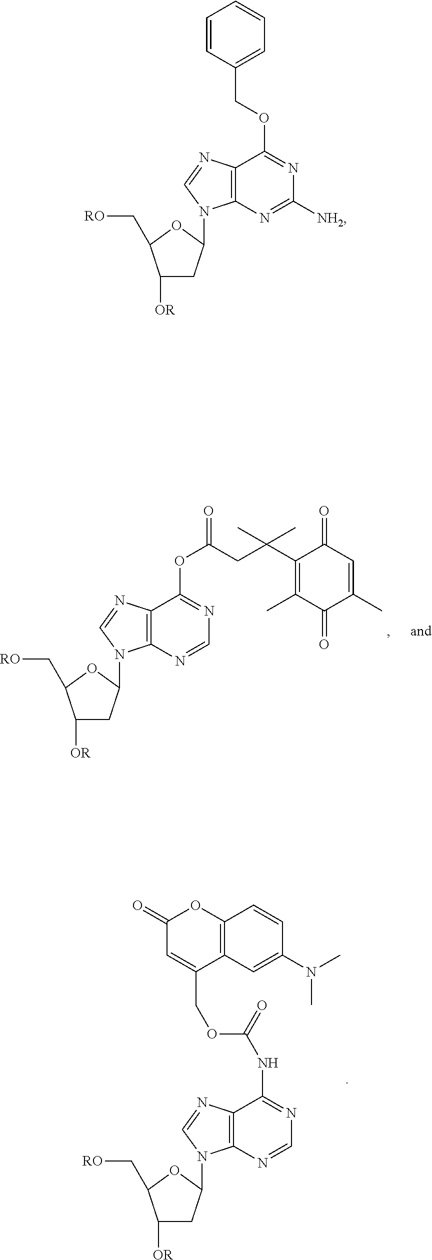

In some embodiments, the convertible residue is selected from the group consisting of O6-guanine, N2-guanine, N7-guanine, N6-adenine, N5-adenine, O4-thymine, N3-thymine, 2-thio-thymine, 4-thio-thymine, N4-cytosine, or N3-cytosine.

In some embodiments, the first state and the second state of the plurality of convertible residues are readable by a sequencing method capable of detecting and differentiating non-naturally occurring and/or modified nucleobases. In some embodiments, the plurality of convertible residues in the first state and the second state are readable by a polymerase enzyme. In some embodiments, the first state and the second state of the plurality of convertible residues are readable by nanopore sequencing. In some embodiments, the first state and the second state of the plurality of convertible residues are readable by sequencing by synthesis. In some embodiments, when the plurality of convertible residues is converted to the second state, properties of the plurality of convertible residues are modified (e.g., having reduced size, altered shape, modified H-bonding, and/or modified polymerase substrate ability) as compared to the first state. In some embodiments, one or more of the plurality of convertible residues are capable of being converted from the second state into a third state; wherein the one or more of the plurality of convertible residues are attached covalently to the nucleic acid polymer in the third state. In some embodiments, each of the plurality of convertible residues is capable of being independently and selectively converted.

In some embodiments, the polymers described herein (e.g., nucleic acid polymers) comprise two or more different sets of convertible residues, each set of convertible residues has a first state and is capable of being converted from the first state into a second state, the first state and the second state being different. In some embodiments, each of the plurality of convertible residues comprises a chemically modifiable moiety that can be modified by light, and the two or more different sets of convertible residues are modifiable by light of a different wavelength. In some embodiments, a first set of convertible residues is modifiable by light of a first wavelength, and a second set of convertible residues is modifiable by light of a second wavelength, the first wavelength and the second wavelength being different.

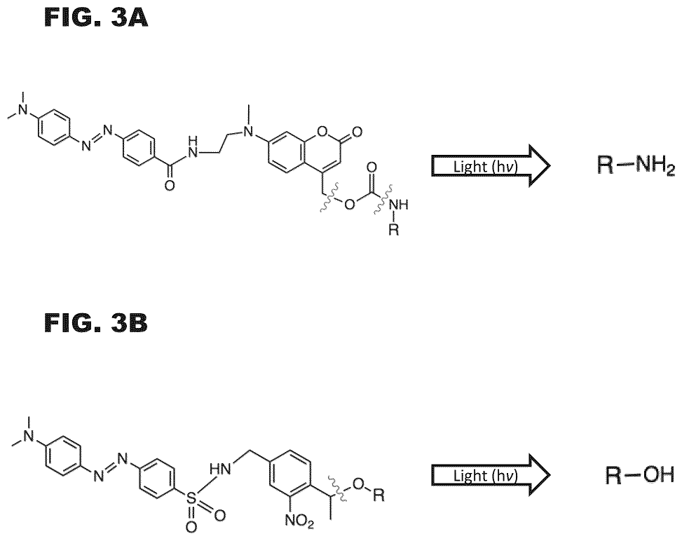

Provided in FIGS. 3A and 3B are examples of releasable quenchers in which the quencher can be released by light. In several embodiments, a quencher can be used with fluorophores such that quenched fluorophores can be utilized in manner akin to “bits” of data, enabling conversion from a first structure state (e.g., quenched) to a second structure state (e.g., unquenched), akin to “0” or “1” digital bit designations. In some embodiments, a quenched fluorophore can be utilized with a second modifiable fluorophore to provide a bit.

The fluorescent state of the bit may be detectable utilizing a nanopore device capable of detecting fluorescence of individual fluorophore molecules, or by a nanopore device that can detect the structural differences in the written bits.

In some embodiments, each convertible residue of a subset of the plurality of convertible residues comprises the quencher.

In some embodiments, the quencher comprises:

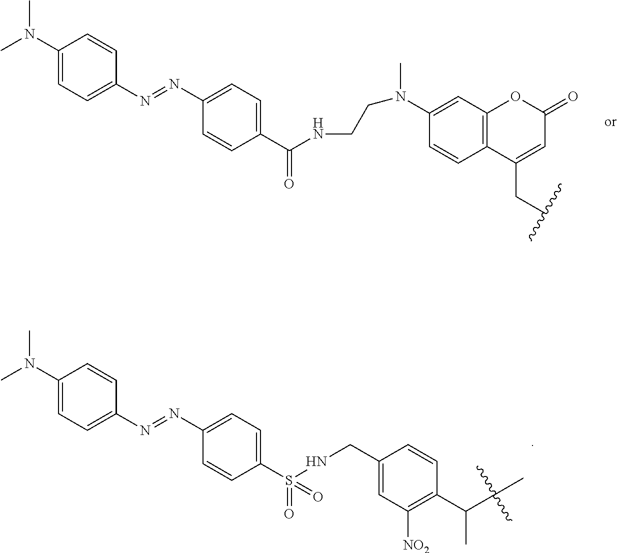 [SMILES: *Cc1cc(=O)oc2cc(N(C)CCNC(=O)c3ccc(/N=N/c4ccc(N(C)C)cc4)cc3)ccc12 ; *C(C)c1ccc(CNS(=O)(=O)c2ccc(/N=N/c3ccc(N(C)C)cc3)cc2)cc1[N+](=O)[O-]]

Each of the releasable quenchers provided in FIGS. 3A and 3B are examples of quenchers that can be released by localized pulses of light, such that when used in conjunction with a nearby fluorophore results in the fluorophore providing detectable fluorescence. Various quenchers groups may be incorporated into caged fluorophores (see, e.g., J. R. Lakowicz, (Ed.). (2013). Principles of fluorescence spectroscopy. Springer science & business media. pp. 277-330 “Quenching of Fluorescence”; and M. K. Johansson, Methods Mol Biol. 2006; 335:17-29; the disclosures of which are each incorporated herein by reference). As can be seen in the figures, each quencher has a linker (e.g., nitrobenzyl group) that may be cleavable with light energy. The quencher may be attached directly to the polymer backbone or to a residue attached to the backbone, as depicted in the FIGS. 3A and 3B, by the R group. While a few examples are provided, it is understood that any appropriate quencher, releasing mechanism, and fluorophore may be used in accordance with the various embodiments.

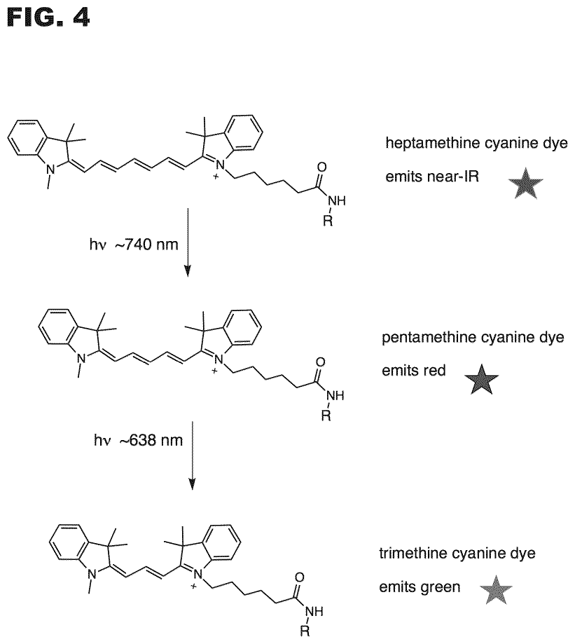

Provided in FIG. 4 provides an example of a photoconvertible fluorophore in which light at particular wavelengths may convert the fluorophore to emit a second and/or third fluorescent wavelength. In several embodiments, a photoconvertible fluorophore can be utilized in manner akin to “bits” of data, enabling conversion from a first structural state (e.g., first emission wavelength) to a second structural state (e.g., second emission wavelength), akin to “0” or “1” digital bit designations. In some embodiments, a photoconvertible fluorophore can be converted from a first emission state (e.g., first emission wavelength) to a second emission state (e.g., second emission wavelength) and further to a third emission state (e.g., third emission wavelength), which allows a ternary code or positively written binary code (i.e., the first emission state is unwritten, the second emission state is akin to “0” and the third emission state is akin to “1”). The fluorescent state of the bit is detectable utilizing a nanopore device capable of detecting fluorescence of individual fluorophore molecules.

In some embodiments, the conversion of the photoconvertible fluorophores from the first structural state into a second structural state is via light pulses at a first wavelength; and wherein the photoconvertible fluorophores are capable of being converted into a third structural state having a third emission wavelength via light pulses at a second wavelength.

In some embodiments, the plurality of convertible residues are capable of being converted by light of a wavelength of 325 nm, 360 nm, 400 nm, 500 nm, 600 nm, 700 nm, or 800 nm. In some embodiments, the plurality of convertible residues are capable of being converted by light of a wavelength of 325 nm, 360 nm, or 400 nm. In some embodiments, the plurality of convertible residues are capable of being converted by light of a wavelength of 325 nm. In some embodiments, the plurality of convertible residues are capable of being converted by light of a wavelength of 360 nm. In some embodiments, the plurality of convertible residues are capable of being converted by light of a wavelength of 400 nm.

In some embodiments, the photoconvertible fluorophore comprises a polymethine cyanine dye. In some embodiments, the photoconvertible fluorophore comprises

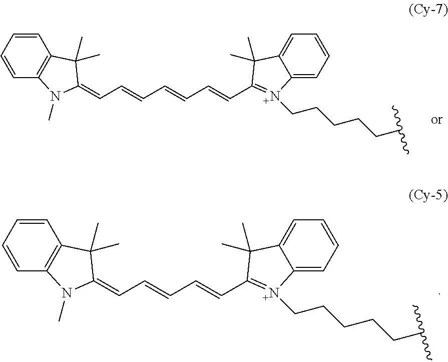 [SMILES: *CCCCC[N+]1=C(/C=C/C=C/C=C/C=C2/N(C)c3ccccc3C2(C)C)C(C)(C)c2ccccc21 ; *CCCCC[N+]1=C(/C=C/C=C/C=C2/N(C)c3ccccc3C2(C)C)C(C)(C)c2ccccc21]

In some embodiments, the photoconvertible fluorophore comprises:

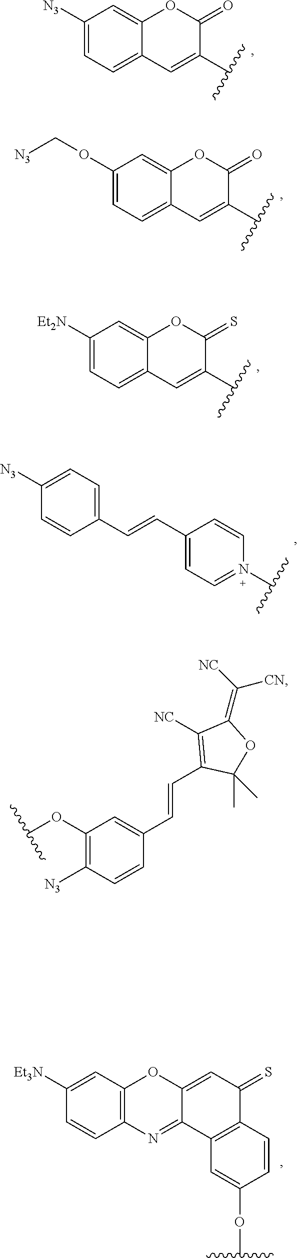

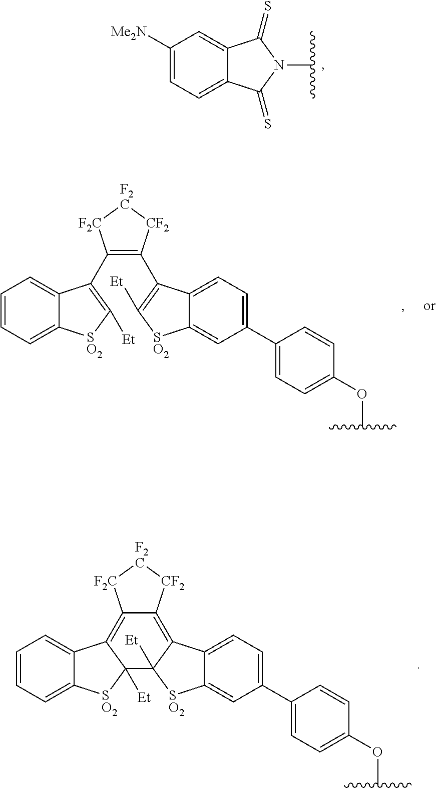

In some embodiments, data writing comprises uncaging of a caged fluorophore, releasing a quencher, releasing a fluorophore, photoinactivation of a fluorophore, photoactivation of fluorophore, bleaching of the fluorophore, or a combination thereof. In some embodiments, wherein the uncaging of the caged fluorophore comprises releasing of a cage from the fluorophore. In some embodiments, the bleaching of the fluorophore comprises bleaching with an additive. In some embodiments, the additive comprises a phosphine.

Various photoconvertible fluorophores groups can be utilized (see, e.g., T. J. Chozinski, L. A. Gagnon, and J. C. Vaughan, FEBS Lett. 2014; 588:3603-12; the disclosure of which is incorporated herein by reference). As can be seen in the example of FIG. 4, a heptamethine cyanine dye can be converted to either a pentamethine cyanine dye or a trimethine cyanine dye by specific wavelengths of light. In this example, the heptamethine cyanine dye having a 7-carbon alkene-containing chain emits near-IR fluorescence. Light energy at approximately 740 nm modifies the alkene-containing chain of the heptamethine cyanine dye resulting in pentamethine cyanine dye having a 5-carbon chain. The pentamethine cyanine dye emits red fluorescence. Furthermore, light energy at approximately 638 nm modifies the alkene-containing chain of the heptamethine cyanine dye or pentamethine cyanine dye resulting in trimethine cyanine dye having a 3-carbon chain. The trimethine cyanine dye emits yellow/green light. Thus, in this approach, data can be encoded as a binary code of red and yellow/green emission signals. The fluorophore can be attached directly to the polymer backbone or to a residue attached to the backbone, as depicted in the figures by the R group. While a few examples are provided, it is understood that any appropriate photoconvertible fluorophore may be used in accordance with the various embodiments.

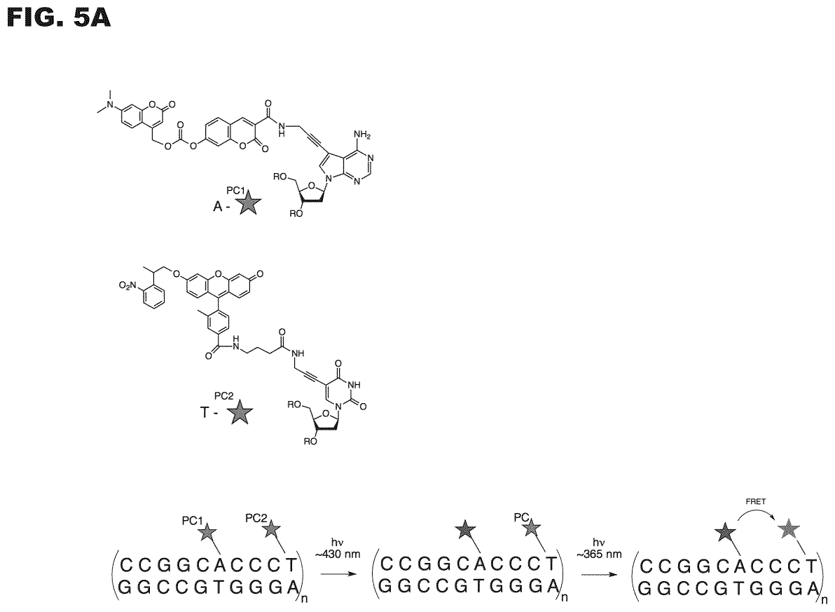

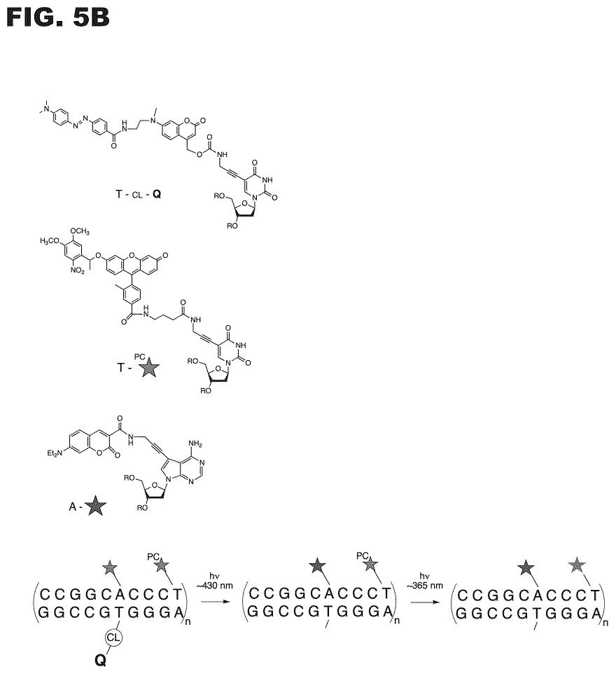

FIG. 5A illustrates an example of a writable two-fluorophore bit utilizing two distinct caged fluorophores. In some embodiments, a two-fluorophore bit may have two fluorophores within approximately 30 angstroms of one another. Exposure of long wavelength light energy to this two-fluorophore bit may result in uncaging of one of the fluorophores, thus resulting in blue florescence. Alternatively, exposure of short wavelength light to this two-fluorophore bit results in uncaging of both of the fluorophores, resulting in a combination of blue and green light. When both fluorophores are uncaged, the blue fluorophore may donate energy to the green fluorophore, resulting in Foerster energy transfer, enhancing the green fluorescence and decreasing the blue florescence. By utilizing two different wavelengths, each resulting in a unique fluorescence signal, a binary code can be generated to store data. It is understood that digital assignment of zero and one is arbitrary and either fluorescent signal can correspond to “zero” or “one”.

FIG. 5B illustrates an example of a writable two-fluorophore bit utilizing a combination of a releasable quencher and a caged fluorophore. In some embodiments, the two-fluorophore bit comprises two fluorophores within approximately 30 angstroms of one another. In some examples, each bit utilizes two fluorophores of distinct color: unmodified coumarin and the photocaged (PC) Tokyo green. In addition, a photoreleasable fluorescence quencher (Q) with photocleavable linker (CL) is also incorporated near the two dyes. Before data writing, the polymer may have little or no fluorescence emission due to the fluorescence quencher and the caging of one fluorophore. To write a bit of data, either a single pulse of approximately 430 nm light may be employed to release the fluorescence quencher, resulting in blue fluorescence from the unmodified coumarin, or dual pulses of ˜430 nm and ˜365 nm are employed to release the quencher release and uncage the Tokyo green, resulting in a coumarin blue plus green Tokyo green emission signal. Alternatively, a single pulse of ˜365 nm light may be sufficient to release the fluorescence quencher and uncage the Tokyo green fluorophore. When both fluorophores are unquenched and uncaged, the blue fluorophore can donate energy to the green fluorophore, resulting in Foerster energy transfer, enhancing the green florescence and decreasing the blue florescence. Thus, in this approach, data may be encoded as a binary code of blue and green emission signals in sequence along the polymer.

Numerous embodiments are also directed to a writable polymer (especially biological polymers) further incorporating one or more of spacers, delimiters, and data tags. In accordance with various embodiments, a spacer may be a residue incorporated within a writable polymer that provides a requisite space between fluorophore bits in accordance with spatial resolution of the data writing mechanism. In many embodiments, a spacer may lack fluorescence capability such that the spacer may not interfere with the ability to read the fluorophore bits. In some embodiments, a spacer may be unreactive with the data writing mechanism. In some embodiments, a writable nucleic acid polymer may utilize the same residue repeatedly for each and every spacer. In some embodiments, however, a writable nucleic acid polymer may utilize two or more different residues as spacers. Any appropriate residue lacking ability to fluoresce may be utilized as spacers, including naturally occurring nucleobases, unnatural nucleobases, tetrahydrofuran abasic residues, and/or ethylene glycol residues.

A delimiter, in accordance with various embodiments, may be a residue that signifies a boundary. In some embodiments, a delimiter may be utilized to separate two adjacent data fields. Any appropriate residue lacking ability to fluoresce may be utilized as a delimiter, including naturally occurring nucleobases, unnatural nucleobases, tetrahydrofuran abasic residues, and/or ethylene glycol residues.

In several embodiments, a data tag may be a string of monomers (typically 4 or more residues) that signifies certain data. For instance, a data tag can signify type of data, date, data source, or any other information. Any appropriate residues lacking ability to fluoresce may be utilized as data tag residues, including naturally occurring nucleobases, unnatural nucleobases, tetrahydrofuran abasic residues, and/or ethylene glycol residues.

Writable polymers can be generated by any appropriate method for generating long polymers. Generally, organic and inorganic polymers are generated by an appropriate polymerization chemistry that controllably produces a linear polymer, including (but not limited to) free radical polymerization, group transfer polymerization, ring opening metathesis polymerization, RAFT polymerization, and condensation polymerization (see, e.g., S. K. Samal, et al., Chem Soc Rev. 2012; 41:7147-94; S. L. Baker, et al., Biomacromolecules. 2019; 20:2392-240; and P. Strasser and I. Teasdale, Molecules. 2020; 25:1716; the disclosures of which are each incorporated herein by reference). Methods for incorporating fluorescent labels into polymers are known to those familiar with the art (see, e.g., W. Zhaoqiang and M. Lingzhi, Progress in Chemistry. 2007; 19:1381-1392; and E. K. Riga, et al., Macromol Chem Phys. 2017; 218:1700273; the disclosures of which are each incorporated herein by reference). Modifiable fluorescent labels can be incorporated by including them in the monomer(s) of the polymer during polymerization, or they may be attached subsequently. For example, if a polymer after synthesis contains a reactive thiol group in a repeating sequence, it can be reacted with maleimide-conjugated alterable fluorophore.

FIGS. 6A to 6C provide examples of organic repeating polymers that contain an iteratively repeated charged group for solubility and mobility through a nanopore. These exemplary polymers further incorporate modifiable fluorophores for writing data. The exemplary structures are not to be interpreted as limiting. Any polymer having one or more charged groups per repeating unit for nanopore mobility and incorporation of one or more fluorescent activatable dyes in the repeating unit can be utilized in accordance with several embodiments. In various embodiments, an organic polymer comprises between 100 and 5000 monomers, as dependent on the method of synthesis and monomer type. In some embodiments, an organic polymer may be a homopolymer. In some embodiments, an organic polymer may be an alternating copolymer.

For biological polymers, in accordance with various embodiments, polymerase extension or chemical synthesis is utilized to generate writable biological polymers. If polymerase extension is utilized, appropriate nucleotides, nucleobases and residues that can be polymerized by the polymerase are to be utilized. If chemical synthesis is utilized, a broader range of nucleobases and residues can be utilized, but generally synthesis results in shorter nucleic acid strands (e.g., between 10 and 200 residues), which can be ligated together to generate longer polymers. It is understood that both polymerase and ligation methods can construct repeating writable nucleic acid polymers in either single-stranded or double-stranded states.

### Modes of Data Writing and Reading

Several embodiments are directed towards writing and reading data on polymers utilizing modifiable fluorophores. In many embodiments, a writable polymer may be provided having modifiable fluorophores and charges iteratively spaced along the polymer.

The provided writable polymer may also have spacers, delimiters, and data tags, as described herein. To write data upon a polymer, in accordance with various embodiments, an individual strand is passed through a device having a nanopore. Various nanopore devices can enable the photoexcitation of single fluorophores as they pass through the pore (see, e.g., D. Garoli, et al., Nano Lett. 2019; 19:7553-7562; and M. Rahman, et al., Lab Chip. 2021; 21:3030-3052; the disclosures of which are each incorporated herein by reference). Photoexcitation of photocages or cleavable linkers results in breaking chemical bonds to yield a binary code of fluorescent bits. Such devices can include structures near the nanopore that amplify light energy to highly localized positions. Examples include (but are not limited to) plasmonic amplifiers such as metallic bowties, metallic nanorods, or zero mode waveguides (see, e.g., J. D. Spitzberg, et al., Adv Mater. 2019; 31:e1900422, the disclosure of which is incorporated herein by reference). Shining light on such plasmonic structures can yield resolution of as small as one nanometer. Alternatively, the use of two lasers can be employed via the STED technique to achieve highly localized illumination (see, e.g., S. J. Sahl and S. W. Hell High-Resolution 3D Light Microscopy with STED and RESOLFT. 2019 Aug. 14. In: Bille J F, editor. High Resolution Imaging in Microscopy and Ophthalmology: New Frontiers in Biomedical Optics. Chain (CH): Springer; 2019. Chapter 1, the disclosure of which is incorporated herein by reference).

In some embodiments, modification of fluorophores may not be required to be performed at every repeating unit of the polymer. Units may be skipped during the writing process. When reading polymers with skipped units, such skipped units can be interpreted as blank or null and the reader progresses to the next fluorescent position to find the next bit in the string. Skipping of a repeating unit during writing and reading may be performed intentionally to space out bit writing to best suit the resolution of the writing method; skipping may also occur in random fashion due to stochastic movements and alteration of the rate of the molecule passing through the pore.

In many embodiments, the writing device is provided a software code for writing the data into the polymer. Accordingly, the writing device, directed by this code, will control pulses of light by time and/or wavelength to selectively modify the fluorophores or quenchers of the polymer to yield a data code (e.g., binary code). After writing a code of data into the polymer, it can be stored in the dark and by any appropriate means for maintaining integrity of the polymeric molecule. For instance, data written polymers can be stored dry, as a precipitate, or in an appropriate solution at room temperature, or at colder temperatures (e.g., −20 C). Stabilizers such as (for example) alcohol, antioxidants, chelating agents and biological inhibitors (e.g., nuclease inhibitors or protease inhibitors), may be included with the stored polymer.

Polymers most efficiently store data at the single molecule level, providing the highest potential density of information. In some embodiments, however, if redundancy of data is required for better accuracy of data storage, then a plurality of polymers could be used to redundantly write the same data on each polymer of the plurality. Error correction algorithms are already well developed for digital data storage, and some of these algorithms can be applied in the present approach (see J. Li, et al., IEEE Transactions on Emerging Topics in Computing. 2021; 9:651-663, the disclosure of which is incorporated herein by reference).

Highly localized light excitation can be achieved via specialized sub-wavelength microscopic focusing strategies such as STEDX, or by the use of nanoplasmonic structures such as bow ties or by the use of zero-mode waveguides (see Y. Fang and M Sun, Light Sci Appl. 2015; 4:e294; and X. Shi, et al. Small. 2018; 14:e1703307; the disclosures of which are each incorporated herein by reference). Timing of light pulses and controlled passage of the writable polymer can be in concert with appropriate spacing such that data is encoded with fidelity.

In many embodiments, to read the data on written nucleic acid polymers, any appropriate nanopore capable of reading fluorescence of single fluorophores or capable of analyzing structural differences in monomers can be utilized. In certain embodiments, a device is capable both of writing and reading nucleic acid or organic polymers. In certain embodiments, a single nanopore has dual functionality for both writing and reading polymers, however, some devices may include distinct nanopores for performing writing and reading.

Various nanopore devices are available, or alternatively, a nanopore device can be fabricated or manufactured for writing and/or reading the data. The nanopore can be comprised of solid-state materials.

In addition to utilizing a nanopore device for reading, a polymer may be stretched and imaged utilizing a device capable or reading fluorophores along stretched polymers. Polymer (especially DNA) stretching or combing methods are known to practitioners of the art (see, e.g., Z. E. Nazari and L. Gurevich, J. Self-Assembly and Molecular Electronics 2013; 1:125-148; A. Kaykov, et al., Sci Rep. 2016; 6:19636; the disclosures of which are incorporated herein by reference). Alternatively, in some embodiments, super resolution microscopy can be employed (via the STED approach or other known methods) for achieving high spatial resolution. Imaging of a single DNA strand by STED can yield a sequence of dye colors, which represents a string of bits. In some embodiments, the method may be automated for high throughput, with many strands stretched in one field of view, and automated imaging software that reads a string of dyes (bits) and converts it into digital information.

In some embodiments, the use of light to convert or otherwise alter dyes and/or quenchers in a repeating polymer may result in detectable structural changes in the repeating units of the polymers. Accordingly, in addition to the above fluorescence methods, written polymers can also be read by non-fluorescence methods. In some embodiments, nanopore sequencing by the use of alterations of ion flow may be used. Another method involves the reading of optical absorption or other spectral signatures in the altered dyes (such as vibrational modes) as the strand passes though the pore.

In some embodiments, the plurality of converted residues are read in sequence.

In some embodiments, the plurality of converted residues are read in sequence from one end of the polymer to the other end of the polymer encoded with data.

### Methods of Installing Bits

Described herein are compositions and systems for data storage utilizing polymers, and methods of installing data bits and methods of use, in accordance with various embodiments. In several embodiments, a system of data storage comprises writable polymers having one or more sets of chemically modifiable structures that have been installed onto the polymer. In some embodiments, the chemically modifiable structures may be modifiable fluorophores. Accordingly, a writable polymer is akin to a blank tape that is encodable, wherein the writable polymer is encoded by modifying the sets of modifiable structures along the polymer strand, which can be done by any writing method for modification, including (but not limited to) localized light or redox energy. In many embodiments, a set comprises one or more chemically modifiable structures that can be modified to encode a bit of data. In various embodiments, a chemically modifiable structure may be a caging group (e.g., cage or photo-removable group), a quencher, a photoconvertible fluorophore, or a stable secondary structure in a nucleic acid. Accordingly, in various embodiments, a chemically modifiable structure may be modified by uncaging, releasing a quencher, photoconverting a fluorophore, or altering the stable secondary structure of the nucleic acid. Modification of sets of one or more chemically modifiable structures can be thought of as a data code, where a set of one or more chemically modifiable structures may be akin to a “bit;” one structural state of the set is akin to a “0,” and a second structural state of the set is akin to a “1”. For instance, in one example, a set of chemically modifiable structures can comprise a caged fluorophore and quenched fluorophore; releasing only the quencher can be akin to “0” and releasing the quencher and uncaging the caged fluorophore can be akin to a “1”. It should be understood, however, that a binary code is not the only possibility, and data codes can be written in ternary, quaternary, or other numeral system code, which can be done utilizing multiple types of chemically modifiable structures or performing multiple writings/modifications. The modification of a set of one or more chemically modifiable structures can be stable, or permanent, which allows for long-term archiving, especially if kept in a dark storage location.

In many embodiments, a polymer incorporates a reactive group iteratively spaced along the polymer, which can be utilized to install sets of one or more chemically modifiable structures via a bonding reaction. Likewise, in several embodiments, each set of one or more chemically modifiable structures can include a reactive group, which can be utilized to bond with one of the iteratively spaced reactive groups of the polymer such that each set can be installed. In many embodiments, installation of the sets of one or more chemically modifiable structures onto the polymer converts the polymer into a writable polymer capable of encoding data via the sets of one or more chemically modifiable structures.

Many embodiments are directed towards compositions of writable polymers. Any appropriate polymer can be utilized, including (but not limited to) biological polymers, organic polymers, and inorganic polymers. Biological polymers (and their analogues) include (but are not limited to) DNA, RNA, phosphorothiate DNA, glycerol nucleic acids (GNA), threose nucleic acids (TNA), 2′-fluoro-DNA 2′-O-methyl RNA, locked nucleic acids (LNA), peptide chains, and peptoid chains. A nucleic acid polymer may be single stranded or double stranded. Further, a nucleic acid polymer may utilize any enantiomer (e.g., d-DNA, 1-DNA). Similarly, a peptide polymer can be any enantiomer.

In several embodiments, the polymer may incorporate a charged group iteratively spaced along the polymer. In some embodiments, a polymer may be iteratively charged via a charged backbone. In some embodiments, a polymer may be iteratively charged by incorporating monomers having a charged constituent. In various embodiments, iterative charge of a polymer may be provided by negatively charged phosphate groups, negatively charged sulfate groups, negatively charged carboxylate groups, or positively charged ammonium groups.

In several embodiments, a writable nucleic acid polymer comprises a plurality of sets of one or more chemically modifiable structures that are linked by a polymer backbone. In certain embodiments, the sets of one or more chemically modifiable structures may be spaced apart to provide spatial resolution such that each set can be independently and selectively modified in accordance with data encoding. In some embodiments, spacer residues linked via the polymer backbone are utilized to provide spaces between the modifiable fluorophore bits. In various embodiments, a writable nucleic acid polymer can further include delimiters and/or data tags for labeling or locating the data.

In several embodiments, a writing procedure is utilized to encode a writable polymer with data. Data encoding can be performed by selectively modifying the sets of one or more chemically modifiable structures of a polymer such that the written polymer contains a sequence of structural modifications, akin to a binary code of “zeros” and “ones”.

In some embodiments, the plurality of convertible residues are selectively modified in sequence.

In some embodiments, the plurality of convertible residues are selectively modified in sequence from one end of the polymer to the other end of the polymer.

In accordance with many embodiments, data written polymers are stored in the dark free of photobleaching light. In some embodiments, data written polymers are stored in environments that exclude air or oxygen, which may enhance stability. Stabilizers such as (for example) alcohol, antioxidants, chelating agents and biological inhibitors (e.g., nuclease inhibitors or protease inhibitors), may be included with the stored polymer. To read the data on written nucleic acid polymers, a nanopore capable of analyzing structural differences in monomers can be utilized, such as Oxford Nanopore Technologies PromethION, MinION, and GridION sequencing platforms (Oxford, UK). Also, a nanopore device can be fabricated or manufactured for reading the data. The nanopore can be comprised of solid-state materials, or can contain one or more proteins. Alternatively, when fluorophores are utilized in the sets of one or more chemically modifiable structures, any appropriate nanopore capable of detecting fluorescence of the fluorophores can be utilized, such as Pacific Bioscience's Single Molecule, Real-Time (SMRT) sequencing platform (Menlo Park, CA).

This disclosure provides methods to install chemically modifiable structures onto existing polymers, such as, for example, long organic polymers and/or DNA, in accordance with various embodiments. The installation of chemically modifiable structures can enable these polymers to have data written into them via localized light or redox signals. These “writable” groups can be iteratively added along the polymer before data writing rendering the polymeric molecule into a data encodable polymer. Localized optical or electronic signals can be utilized along the polymer at the iterative groups to chemically modify these writable groups into modified structural states akin to a binary code of ones and zeros that can be read by fluorescence imaging or by time-resolved current measurements. This approach makes it possible to encode data in very long polymers, achieving high data density and providing a mechanism for rapid data writing.

Installation of Modifiable Data Bits onto Polymers to Store Data

Compounds and methods of synthesis in accordance with embodiments of the disclosure are based on installing a plurality of sets of one or more chemically modifiable structures onto a pre-existing polymer to generate a data encodable polymer. A set of one or more chemically modifiable structures can be added to a monomer to create a data “bit,” such that polymer can be encoded in a code (e.g., binary code). Each set of chemically modifiable structures (i.e., bit) can exist in two or more states, an unwritten state, and at least a first written state, and in some embodiments, additional written states. In some embodiments, the unwritten state is akin to a “zero” in binary code and the written state is akin to “one.” In some embodiments, a first written state is akin to a “zero” in binary code and a second written state is akin to “one.” Writable polymers can be generated having long lengths (e.g., 5 to 100,000 monomers with added sets of chemically modifiable structures, or more) and can be produced in bulk, prior to data writing.

In several embodiments, a writable polymer comprises a plurality of monomers with added sets of chemically modifiable structures that may be linked by the polymer backbone. In certain embodiments, the sets of chemically modifiable structures may be iteratively spaced apart to provide spatial resolution such that each set of chemically modifiable structures can be independently modified. The spatial resolution depends, at least in part, on the writing mechanism. For instance, if an optical light source and device with 1 nm of resolution is used to modify chemically modifiable structures, then each set of chemically modifiable structure bits can be separated by at least 1 nm. Any appropriate spacer between the fluorophore bits can be utilized. In some embodiments, residues linked by the polymer backbone can be utilized as spacers. In some embodiments in which nucleic acids are utilized as the polymer, spacers may be nucleobases without an installed set of chemically modifiable structures. In various embodiments, a writable polymer may further include delimiters and/or data tags for labeling the data, each of which may be provided by a particular sequence of residues.

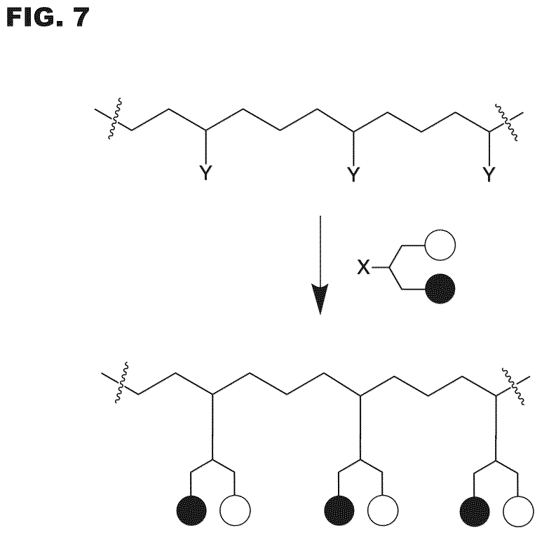

FIG. 7 provides an exemplary schematic for installing a plurality of chemically modifiable structures to a polymer to generate a writable polymer in accordance with various embodiments. In several embodiments, chemically modifiable structures are modifiable by light or redox signals into at least a second distinct state to yield a data code (e.g., a binary code akin to “0” and “1”). Shown in FIG. 7 is a generic polymer chain with repeating monomers that comprise a reactive group Y. Note that the polymer chemical structure shown here is generic, and represents virtually any polymer, including DNA. Y denotes a reactive group that may be incorporated into the polymer, which can be incorporated during polymer synthesis, and X denotes a complementary reactive group on the chemically modifiable structures. X may react with Y to form a chemical bond to yield iteratively spaced chemically modifiable structures along the polymer. In many embodiments, data may be written by providing a localized light or redox signal sequentially along the strand, such as (for example) by superresolution laser excitation (S. J. Sahl and S. W. Hell High-Resolution 3D Light Microscopy with STED and RESOLFT. 2019 Aug. 14. In: Bille J F, editor. High Resolution Imaging in Microscopy and Ophthalmology: New Frontiers in Biomedical Optics. Chain (CH): Springer; 2019. Chapter 1, the disclosure of which is incorporated herein by reference), or by the use of a nanopore device (D. Garoli, et al., Nano Lett. 2019; 19:7553-7562; and M. Rahman, et al., Lab Chip. 2021; 21:3030-3052; the disclosures of which are each incorporated herein by reference), or (for DNA) by a sensitized protein that progresses along the strand (see Pat. Appl. No. 63/248,407, the disclosure of which is incorporated herein by reference).

In various embodiments, polymers for installing sets of chemically modifiable structures can be any length, for example, from as short as 15 monomers to longer than 5000 monomers in most organic polymers, to longer than 100,000 monomers in nucleic acid polymers. In various embodiments, a polymer is greater than 100 monomers long, is greater than 200 monomers, is greater than 300 monomers, is greater than 400 monomers, is greater than 500 monomers, is greater than 1000 monomers, is greater than 5000 monomers, is greater than 10,000 monomers, is greater than 50,000 monomers, or is greater than 100,000 monomers. Maximum lengths are only limited by the stability of the selected polymer type, by the method used to make them, and by the method used to read the written data. Longer strands containing more bits have the advantage of containing more data per molecule.

Several embodiments are directed to sets of one or more chemically modifiable structures, which can be incorporated into an existing polymer to create a writable polymer. A chemically modifiable structure, in accordance with various embodiments, comprises one or more chemical groups that are capable of being structurally altered from a first structure state (e.g., caged) into a second structural state (e.g., uncaged) by controlled reaction chemistry (e.g., light energy or redox energy). In some embodiments, a chemically modifiable structure incorporates a fluorophore that may be caged, where the caged fluorophore is a first structural state and release of the cage results in a second structural state. In some embodiments, a chemically modifiable structure incorporates a quenched fluorophore, where the quenched fluorophore has a first structural state and release of the quencher results in a second structural state, may result in brighter fluorescence. In some embodiments, a chemically modifiable structure incorporates a fluorophore capable of being converted from a first state having a particular fluorescent quality (e.g., a first emission or absorption wavelength) into a second state having a second distinguishable fluorescent quality (e.g., a second emission or absorption wavelength). In some embodiments, a chemically modifiable structure incorporates two structurally alterable groups to provide a bit that is writable into at least two different states.

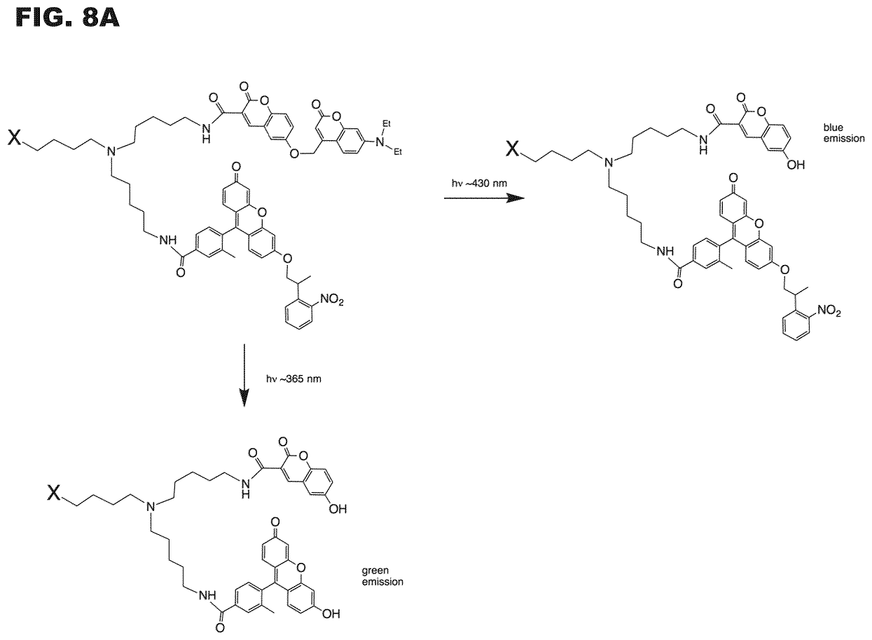

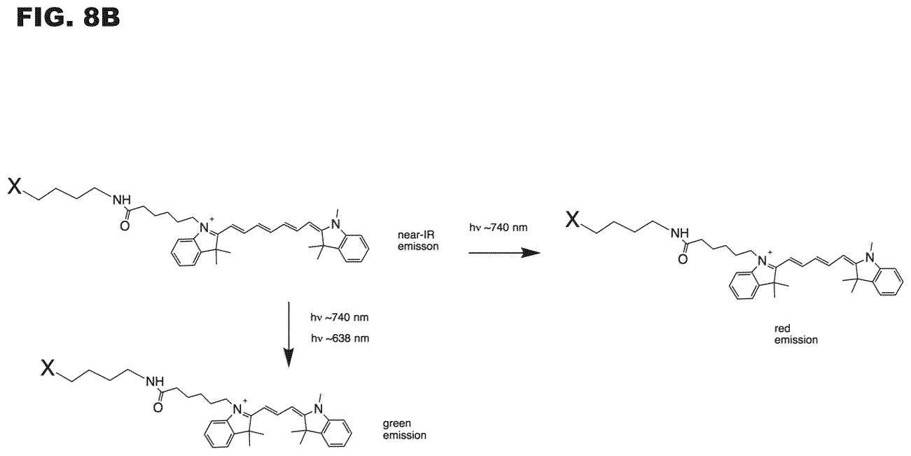

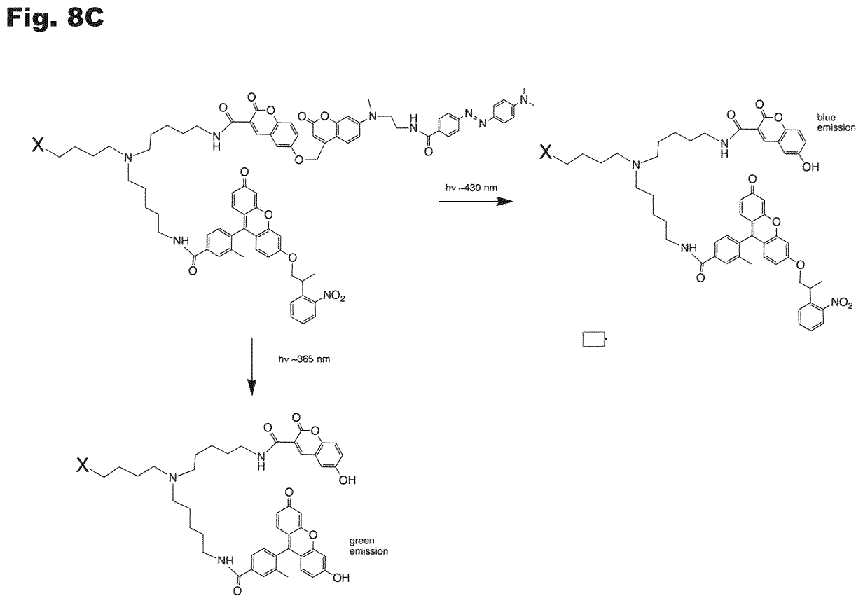

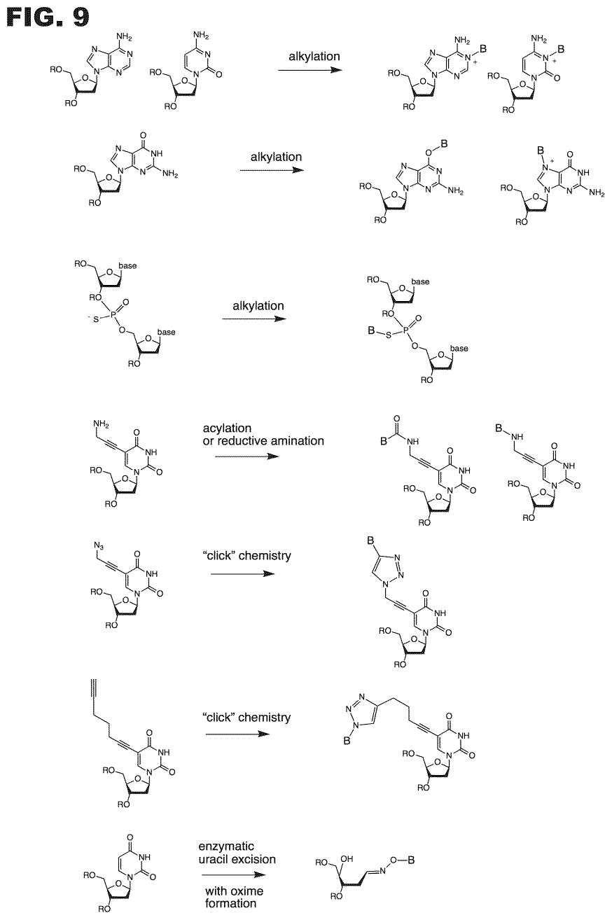

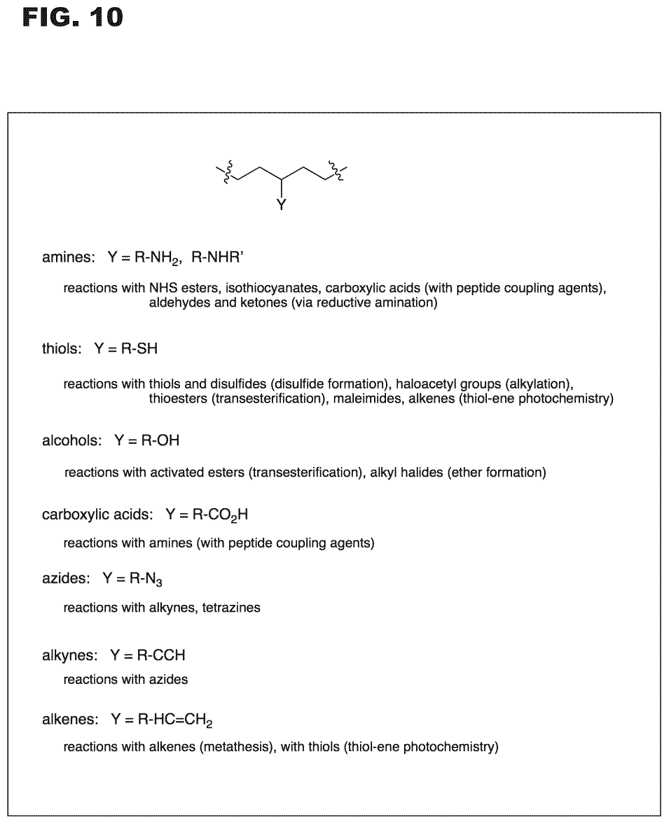

FIG. 8A to 8C each provide an example of sets of chemically modifiable structures that can be utilized as writable “bits,” in accordance with various embodiments. In many embodiments, the of sets of chemically modifiable structures can be added along the polymer strand. As shown in FIG. 8A to 8C, these sets of chemically modifiable structures have the capacity for dual bit writing. Accordingly, each set of chemically modifiable structures is added to a monomer in an unwritten state and then converted into a first written state and/or a second written state. The “X” denotes a reactive group that can form a covalent bond with polymer monomer. The polymer can be DNA or modified DNA (see FIG. 9), or a polymer with reactive groups (see FIG. 10).

The example in FIG. 8A is set of chemically modifiable structures that comprises two caged fluorophores. In some embodiments, the two-fluorophore bit comprises two fluorophores within approximately 30 angstroms of one another. In this example, two fluorophores of distinct color are utilized in the set of chemically modifiable structures: coumarin and Tokyo green. In addition, each fluorophore may be caged with a caging group. Before data writing, the polymer may have little or no fluorescence emission due to the caging of the fluorophores. to write a bit of data, one or more pulses of violet light (e.g., approximately 430 nm) light may be employed to singularly uncage a blue coumarin fluorophore, resulting in blue fluorescence. Alternatively, one or more pulses of UV light (e.g., approximately 365 nm) may be utilized to uncage both the blue coumarin fluorophore and the Tokyo green fluorophore, resulting in a coumarin blue plus green Tokyo green emission signal. Or alternatively, dual pulses of violet light (e.g., approximately 430 nm) and UV light (e.g., approximately 365 nm) uncage both the blue coumarin fluorophore and the Tokyo green fluorophore, resulting in a coumarin blue plus green Tokyo green emission signal. In some embodiments, where both fluorophores where may be uncaged, the blue fluorophore may donate energy to the green fluorophore, resulting in Forster energy transfer, enhancing the green florescence and decreasing the blue florescence. Thus, in this approach, data may be encoded as a binary code of blue and green emission signals in sequence along the polymer. In some embodiments, the set of chemically modifiable structures can be installed onto the polymer via a reactive residue attached to the backbone, as depicted in the FIGS. 8A-8C by the X. While a few examples are provided, it is understood that any appropriate fluorophores and cages may be used in accordance with the various embodiments.

Various photo removable groups can be incorporated into caged fluorophores (see, e.g., Y. Zhao, et al., J Am Chem Soc. 2004; 126:4653-63; the disclosure of which is incorporated herein by reference). In some embodiments, each fluorophore may have a caging constituent that is linked by a linker (e.g., ether group) that is cleavable with energy (e.g., light or redox). The fluorophores can be attached directly to the polymer backbone via a reaction group on a residue attached to the backbone. While a few examples are provided, it is understood that any appropriate photo-removable group and fluorophore may be used in accordance with the various embodiments.

The example in FIG. 8B is set of chemically modifiable structures that comprises a photoconvertible fluorophore in which light at particular wavelengths can convert the fluorophore to emit a second and/or third fluorescent wavelength. In several embodiments, a photoconvertible fluorophore can be utilized in manner akin to “bits” of data, enabling conversion from a first structural state (e.g., first emission wavelength) to a second structural state (e.g., second emission wavelength), akin to “0” or “1” digital bit designations. In some embodiments, a photoconvertible fluorophore can be converted from a first emission state (e.g., first emission wavelength) to a second emission state (e.g., second emission wavelength) and further to a third emission state (e.g., third emission wavelength), which allows a ternary code or positively written binary code (i.e., the first emission state is unwritten, the second emission state is akin to “0” and the third emission state is akin to “1”). The fluorescent state of the bit is detectable utilizing a nanopore device capable of detecting fluorescence of individual fluorophore molecules.

Various photoconvertible fluorophores groups can be utilized (see, e.g., T. J. Chozinski, L. A. Gagnon, and J. C. Vaughan, FEBS Lett. 2014; 588:3603-12; the disclosure of which is incorporated herein by reference). As can be seen in the example of FIG. 8B, a heptamethine cyanine dye can be converted to either a pentamethine cyanine dye or a trimethine cyanine dye by pulses of specific wavelengths of light. In some embodiments, the heptamethine cyanine dye having a 7-carbon alkene-containing chain emits near-IR fluorescence. In some embodiments, light energy at approximately 740 nm modifies the alkene-containing chain of the heptamethine cyanine dye resulting in pentamethine cyanine dye having a 5-carbon chain. In certain cases, the pentamethine cyanine dye emits red fluorescence. Furthermore, for some embodiments, light energy at approximately 638 nm may modify the alkene-containing chain of the heptamethine cyanine dye or pentamethine cyanine dye resulting in trimethine cyanine dye having a 3-carbon chain. For some examples, the trimethine cyanine dye may emit yellow/green light. Thus, in this approach, data can be encoded as a binary code of red and yellow/green emission signals. The set of chemically modifiable structures can be installed onto the polymer via a reactive residue attached to the backbone, as depicted in FIG. 8A-8C by the X. While a few examples are provided, it is understood that any appropriate photoconvertible fluorophore may be used in accordance with the various embodiments.

In some embodiments, as depicted in FIG. 8C, a set of chemically modifiable structures may comprise a combination of a releasable quencher and a caged fluorophore. In some embodiments, the two-fluorophore bit comprises two fluorophores within approximately 30 angstroms of one another. In some examples, two fluorophores of distinct color are utilized in the set of chemically modifiable structures: unmodified coumarin and the photocaged Tokyo green. In addition, a photoreleasable fluorescence quencher with a photocleavable linker can be incorporated near the two dyes (and in this example is attached to the coumarin blue fluorophore). In some embodiments, before data writing, the polymer may have little or no fluorescence emission due to the fluorescence quencher and the caging of the fluorophore. To write a bit of data, one or more pulses of violet light (e.g., approximately 430 nm) light may be employed to singularly release the quencher, resulting in blue fluorescence from the blue coumarin. Alternatively, one or more pulses of UV light (e.g., approximately 365 nm) may be utilized to release the quencher and uncage the Tokyo green fluorophore, resulting in a coumarin blue plus green Tokyo green emission signal. Or alternatively, dual pulses of violet light (e.g., approximately 430 nm) and UV light (e.g., approximately 365 nm) may release the quencher and uncage the Tokyo green fluorophore, resulting in a coumarin blue plus green Tokyo green emission signal. When both fluorophores are unquenched and uncaged, the blue fluorophore may donate energy to the green fluorophore, resulting in Forster energy transfer, enhancing the green florescence and decreasing the blue florescence. Thus, in this approach, data can be encoded as a binary code of blue and green emission signals in sequence along the polymer. The set of chemically modifiable structures can be installed onto the polymer via a reactive residue attached to the backbone, as depicted in FIGS. 8A-8C by the X. While a few examples are provided, it is understood that any appropriate fluorophores and cages may be used in accordance with the various embodiments.

Various quencher groups can be utilized (see, e.g., J. R. Lakowicz, (Ed.). (2013). Principles of fluorescence spectroscopy. Springer science & business media. pp. 277-330 “Quenching of Fluorescence”; and M. K. Johansson, Methods Mol Biol. 2006; 335:17-29; the disclosures of which are each incorporated herein by reference). In some embodiments, a quencher has a linker (e.g., nitrobenzyl group) that is cleavable with light energy. The quencher can be attached directly to the polymer backbone, to a residue attached to the backbone, or to a fluorophore. While a few examples are provided, it is understood that any appropriate quencher, releasing mechanism, and fluorophore may be used in accordance with the various embodiments.

FIG. 9 provides examples of reactions with nucleic acid and modified nucleic acid polymers that, for some embodiments, may be used to install sets of chemically modifiable structures (e.g., fluorophores). In some embodiments, reactions to install sets of chemically modifiable structures include (but are not limited to) alkylation, acylation, reductive amination, “click” chemistry, and enzymatic uracil excision with oxime formation. While a few examples are provided, it is understood that any appropriate bond-forming reaction may be used in accordance with the various embodiments. Any nucleic acid polymer can be utilized, including (but not limited to) DNA, RNA, phosphorothiate DNA, glycerol nucleic acids (GNA), threose nucleic acids (TNA), 2′-fluoro-DNA 2′-O-methyl RNA, and locked nucleic acids (LNA). A nucleic acid polymer may be single stranded or double stranded. Further, a nucleic acid polymer may utilize any enantiomer (e.g., R-DNA, L-DNA). In many embodiments, sets of chemically modifiable structures are installed on a naturally occurring DNA polymer (e.g., extracted from a biological source).

In many embodiments, polymerase extension or chemical synthesis can be utilized to generate biological polymers. Alternatively, in many embodiments, biological polymers can be extracted from a biological source. If polymerase extension is utilized, appropriate nucleotides, nucleobases and residues that can be polymerized by the polymerase are to be utilized. If chemical synthesis is utilized (e.g., phosphoramidite synthesis), a broader range of nucleobases and residues can be utilized, but generally synthesis results in shorter nucleic acid strands (e.g., between 10 and 200 residues), which can be ligated together to generate longer polymers. It is understood that both polymerase and ligation methods can construct repeating nucleic acid polymers in either single-stranded or double-stranded states.

FIG. 10 provides examples of polymer sidechain functional groups that can react with suitably functionalized sets of chemically modifiable structures. Functional groups that can be utilized include (but are not limited to) amines, thiols, alcohols, carboxylic acids, azides, alkynes, and alkenes. While a few examples are provided, it is understood that any appropriate bond-forming functional group and reaction may be used in accordance with the various embodiments. Any polymer having the available functional groups can be utilized and can be generated by any appropriate method for generating long polymers. Generally, organic and inorganic polymers are generated by an appropriate polymerization chemistry that controllably produces a polymer, including (but not limited to) free radical polymerization, group transfer polymerization, ring opening metathesis polymerization, RAFT polymerization, and condensation polymerization (see, e.g., S. K. Samal, et al., Chem Soc Rev. 2012; 41:7147-94; S. L. Baker, et al., Biomacromolecules. 2019; 20:2392-240; and P. Strasser and I. Teasdale, Molecules. 2020; 25:1716; the disclosures of which are each incorporated herein by reference). For example, if a polymer after synthesis contains a reactive thiol group in a repeating sequence, it can be reacted with maleimide-conjugated sets of chemically modifiable structures.

Any bond-forming reaction can be utilized to install a writable bit onto a polymer. In some embodiments, the bond-forming reaction is a nucleophilic attack by an amine, a thiol, or an alcohol onto an sp3 carbon with a leaving group (e.g., iodide, bromide, chloride, tosylate, triflate, mesylate, or similar). The nucleophile reactive group can be on the polymer with the electrophile on the writable bit, or vice versa.

In some embodiments, the bond-forming reaction is an amide bond formation reaction between an amine and a carboxylic acid group. The amide bond formation can be promoted by a condensing or coupling reagent such as dicyclohexylcarbodiimide, or water soluble carbodiimides such as EDC, or uronium and phosphonium peptide coupling reagents such as PyBOP and HBTU. The carboxylic acid can be on the polymer with the amine on the writable bit, or vice versa.

In some embodiments, the bond-forming reaction is an amide bond formation between an amine and an activated ester. Activated esters include (but are not limited to) NHS ester or thioester. The activated ester can be on the polymer with the amine on the writable bit, or vice versa.

In some embodiments, the bond-forming reaction is a urea or a thiourea formation by reaction of an amine with an isocyanate or isothiocyanate. The isocyanate or isothiocyanate may be on the polymer with the amine on the writable bit, or vice versa.

In some embodiments, the bond-forming reaction is a “click” electrocyclization reaction between an azide and an alkyne, resulting in azide-alkyne cycloaddition. The azide can be on the polymer with the alkyne on the writable bit, or vice versa.

In some embodiments, the bond-forming reaction is a tetrazine cycloaddition between a tetrazine and a strained alkene. The tetrazine can be on the polymer with the strained alkene on the writable bit, or vice versa.

In some embodiments, the bond-forming reaction is oxime or hydrazine formation between an aldehyde or a ketone with an aminooxy group or a hydrazine group. The aldehyde or the ketone can be on the polymer with the aminooxy group or the hydrazine group on the writable bit, or vice versa.

In some embodiments, the bond-forming reaction is reductive amination by a reaction between an amine with an aldehyde or a ketone followed by reduction with borohydride reagents. The aldehyde or the ketone can be on the polymer with the amine on the writable bit, or vice versa.

In some embodiments, the bond-forming reaction is a thiol addition to a Michael acceptor. Michael acceptors include (but are not limited to) maleimides and bromomaleimides. The thiol can be on the polymer with the Michael acceptor on the writable bit, or vice versa.

In some embodiments, the bond-forming reaction is a thiol exchange reaction with disulfides. The disulfides can be on the polymer with the thiol on the writable bit, or vice versa.

In some embodiments, the bond-forming reaction is a thiol addition to an alkene mediated by light (sometimes referred to as “thiol-click” reaction). The thiol can be on the polymer with the alkene on the writable bit, or vice versa.

In some embodiments, the bond-forming reaction is a nucleophilic attack by an amine, a thiol, or an alcohol onto an activated sulfonyl species. Sulfonyl species include (but are not limited to) sulfur(VI) exchange substrates, sulfuryl fluorides, and iminosulfur oxydifluorides. The nucleophile reactive group can be on the polymer with the electrophile on the writable bit, or vice versa.

In some embodiments, the bond-forming reaction is a metal-catalyzed reaction between alkenes via metathesis. A first alkene can be on the polymer with a second alkene on the writable bit.

In some embodiments, the bond-forming reaction is a nucleophilic attack by an amine, a thiol, or an alcohol onto an activated aryl group. Aryl groups include (but are not limited to) pentafluorobenzene and para-chloro-acylbenzene. The nucleophile reactive group can be on the polymer with the electrophile on the writable bit, or vice versa.

In various embodiments, an organic polymer comprises between 100 and 5000 monomers, as dependent on the method of synthesis and monomer type. In some embodiments, an organic polymer is a homopolymer. In some embodiments, an organic polymer is an alternating copolymer.

In many embodiments, a polymer is iteratively charged along the polymer, which may help in reading the data of polymer in a nanopore device that utilizes charge to transmit the polymer through the pore. In some embodiments, polymer is iteratively charged by having a charged backbone. In some embodiments, a polymer is iteratively charged by incorporating monomers having a charged constituent. In various embodiments, iterative charge of a polymer is provided by negatively charged phosphate or phosphonate groups, negatively charged sulfate or sulfonate groups, negatively charged carboxylate groups, or positively charged ammonium groups.

Numerous embodiments are also directed to a polymer (especially biological polymers) further incorporating one or more of spacers, delimiters, and data tags. In accordance with various embodiments, a spacer is a residue incorporated within a polymer that provides a requisite space between chemically modifiable structure bits in accordance with spatial resolution of the data writing mechanism. In many embodiments, a spacer will lack ability to attach sets of chemically modifiable structures such that the spacer does not interfere with the installation of chemically modifiable structures to the polymer. In some embodiments, a spacer is unreactive with the type of reaction to install the sets of chemically modifiable structures. In some embodiments, a writable nucleic acid polymer will utilize the same residue repeatedly for each and every spacer. In some embodiments, however, a nucleic acid polymer will utilize two or more different residues as spacers. Any appropriate residue lacking ability to install bits may be utilized as spacers, including naturally occurring nucleobases, unnatural nucleobases, tetrahydrofuran abasic residues, and/or ethylene glycol residues.

A delimiter, in accordance with various embodiments, is a residue that signifies a boundary. In some embodiments, a delimiter is utilized to separate two adjacent data fields. Any appropriate residue lacking ability to install bits may be utilized as a delimiter, including naturally occurring nucleobases, unnatural nucleobases, tetrahydrofuran abasic residues, and/or ethylene glycol residues.

In several embodiments, a data tag is a string of monomers (typically 4 or more residues) that signifies certain data. For instance, a data tag can signify type of data, date, data source, or any other information. Any appropriate residues lacking ability to install bits may be utilized as data tag residues, including naturally occurring nucleobases, unnatural nucleobases, tetrahydrofuran abasic residues, and/or ethylene glycol residues.

In many embodiments, any existing nucleic acid molecule can be utilized as a substrate to add sets of chemically modifiable structures. In some embodiments, a set of chemically modifiable structures is installed onto an existing nucleic acid molecule by a chemical reaction with nucleobase (e.g., see FIG. 9). In some embodiments, polymerase extension is performed on an existing nucleic acid molecule to incorporate residues with a reactive functional group (see e.g., FIG. 10). Any appropriate means to perform polymer extension can be utilized. In several embodiments, when performing polymer extension, one or more nucleoside triphosphates are modified with a functional group such that the functional group is incorporated into the nucleic acid polymer throughout the polymer chain. Incorporation of reactive functional groups permits installation of sets of chemically modifiable structures at each of the functional groups along the polymer.

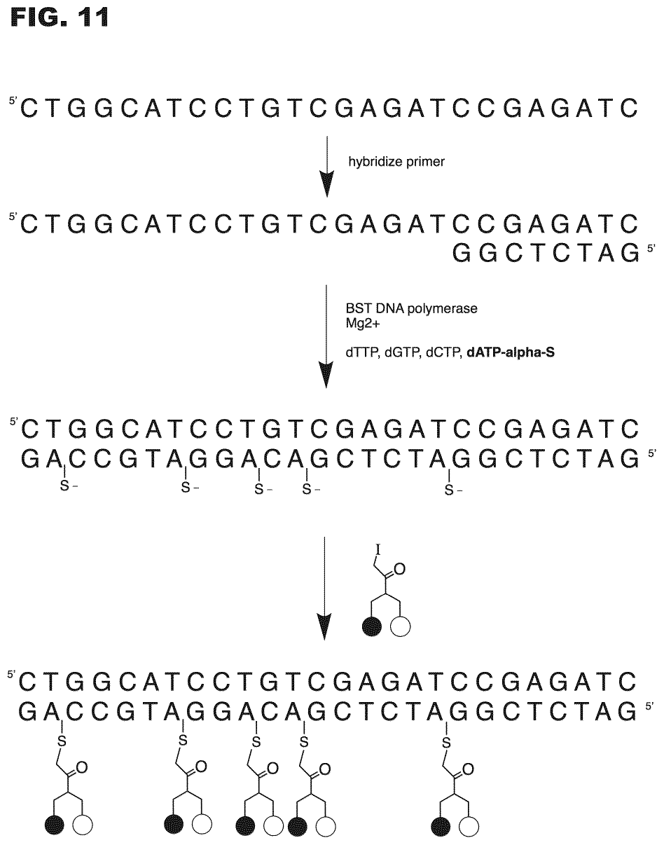

Provided in FIG. 11 is a schematic showing an exemplary approach for converting a naturally-derived DNA molecule into an optically writable polymer via polymer extension and incorporation of alpha-S nucleotides. In some embodiments, DNA can be isolated from cells, and is denatured, resulting in two single strands. A primer complementary to one strand can be added along with a polymerase and nucleoside triphosphates in a polymerase-supportive buffer containing Mg2+. In some embodiments, one or more of the nucleoside triphosphates is an alpha-S nucleotide, thus resulting in a DNA polymer with nucleophilic thiolate reactive groups iteratively spaced along the polymer. Each nucleophilic thioate may provide a means for forming phosphorothioate linkages wherever the reactive group is inserted by the polymerase (see Z. Yang, A. M. Sismour, and S. A. Benner, Nucleic Acids Res. 2007; 35:3118-27, the disclosure of which is incorporated herein by reference). The resulting DNA polymer can be reacted with alkylating groups to install the sets of chemically modifiable structures along the strand. The same process can be applied to non-naturally derived long DNAs such as (for example) repeating DNAs obtained from rolling circle reaction amplification or chemical synthesis with ligation, resulting in more controlled spacing of reactive groups as desired. For more on rolling circle reaction amplification or chemical synthesis with ligation, see Pat. Appl. No. 63/226,720.

Several embodiments are directed to the use of stable and alterable secondary conformations of nucleic acids to delineate data bits. In many embodiments, a data bit can comprise a relatively short nucleic acid string (e.g., between 2 and 200 bases) that can be stably conformed in one, two, or more secondary conformations. In several embodiments, the stable secondary conformations are alterable via light or redox energy. In some embodiments, the stable secondary conformations are locked into its conformation until altered by light or redox energy. To generate stable conformations, in accordance with many embodiments, the nucleic acid polymer incorporates pairs of residues, each residue having an available reactive group such that the residue pairs can form a stable bond with one another. In several embodiments, the pair of residues are within proximity of one another in a secondary conformation such that the reactive groups are able to form the stable bond with one another.

In some embodiments, a data bit comprises one pair of linkable residues capable of two conformations. In some embodiments, where data bits may be capable of two conformations, the two conformations may be a hairpin made stable with bonding between nearby residues in hairpin and simple duplex (i.e., no hairpin). In some embodiments, a data bit comprises two pairs of linkable residues capable of three conformations. In some embodiments of data bits capable of three conformations, the three conformations are a first hairpin made stable with bonding between nearby residues, a second hairpin made stable with bonding between nearby residues, and simple duplex (i.e., no hairpin). In some embodiments of data bits capable of three conformations, the three conformations are a first hairpin and a second hairpin that are each made stable with bonding between nearby residues, only a first hairpin made stable with bonding between nearby residues (i.e., one pair of residues linked to form a stable hairpin and the other pair of residues is have been unlinked into a simple duplex conformation), and simple duplex (i.e., no hairpin). In some embodiments that utilize two pairs of linkable residues to form two hairpins, the two hairpins may be distinguishable from another (e.g., one hairpin is longer than the other hairpin).

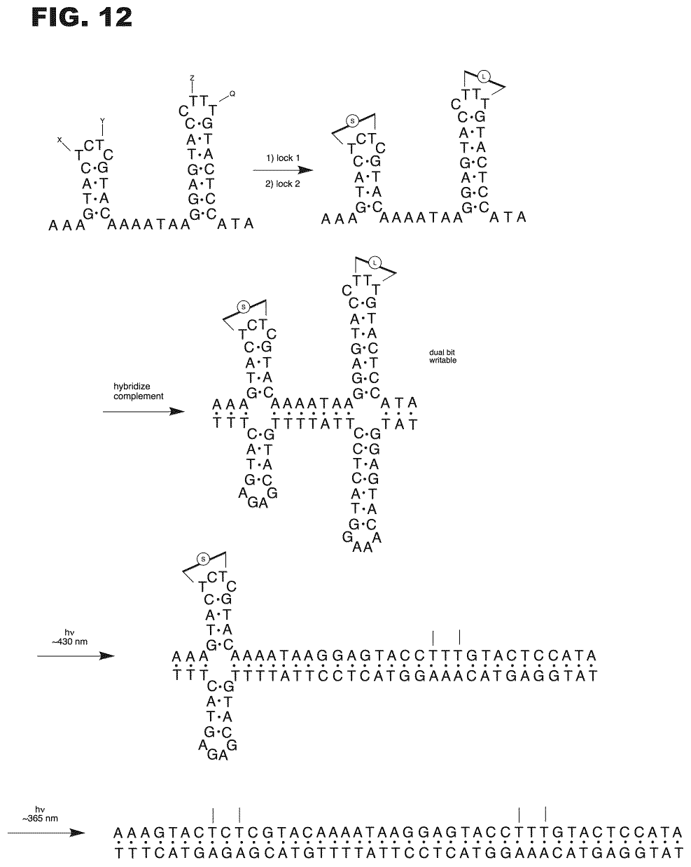

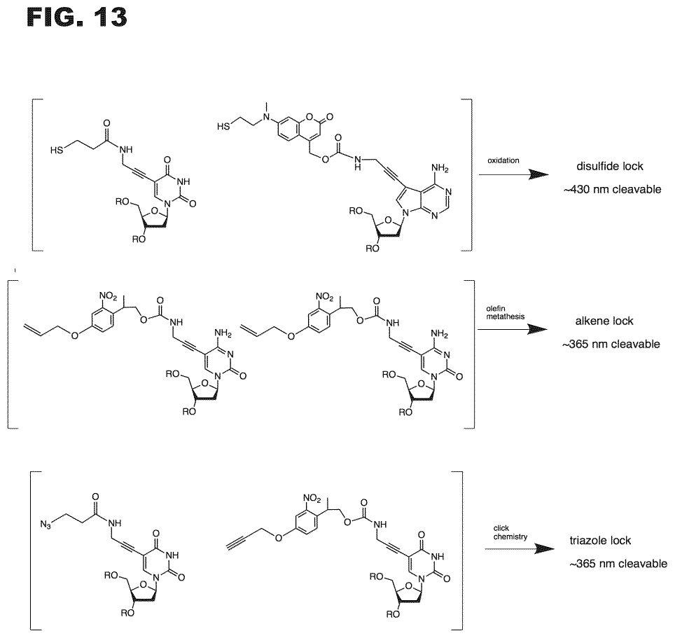

Provided in FIG. 12 is a schematic showing the conversion of a repeating DNA polymer into a stable structurally folded polymer with three alterable, yet stable until writing, structural states, each structural state corresponding to a data code (e.g., binary code). Within the FIG. 12, “X”, “Y”, “Z”, and “Q” denote available reactive groups on residues incorporated into the DNA via oligonucleotide chemical synthesis or via polymerase extension. Examples of such reactive groups are provided in FIGS. 10 and 13. Further, in some embodiments, the reactive groups are provided in pairs that are appropriately spaced to form stable secondary structures (e.g., hairpins). As shown, X and Y, and Z and Q can each act as a pair, and the two pairs can be utilized in conjunction as a data bit. The reactive group X is within two residues of reactive group Y and similarly reactive group Z is within two residues of reactive group Q, but it is to be understood that reactive groups merely need to be within proximity to react with one another when in the secondary conformation but not neighboring in sequence. Further, at least one reactive group of each pair may contain a cleavable linker, such as (for example) a photocleavable linker, to allow release of the reactive group from the residue and. In some embodiments, to formulate stable secondary configurations, the single-stranded folded polymer may be reacted to form bonds between pairs X and Y, and Z and Q (e.g., sulfide bridges as shown in FIG. 12). The pairing may be promoted by the proximity of the reaction groups in the folded hairpins. For some embodiments, the bonding between the nearby residues stably may trap the DNA in the secondary folded state and may destabilize formation of simple duplexes. In some embodiments, the stable secondary conformation can be considered to be a first structural state. Further, as a result of the stable secondary conformation for some embodiments, secondary hairpin structures may be maintained when the DNA polymer strand is hybridized with complementary stand (see FIG. 12). In some embodiments, to write data, light or redox energy can be utilized to selectively cleave a reactive group of one, or both, pairs. As shown in the example of FIG. 12, one or more pulses of violet light energy (e.g., approximately 430 nm) may result in cleaving a functional group of the Z and Q pair, resulting in an “unlocking” of the link between the Z and Q, and alteration of the hairpin formation to a simple duplex.

On the other hand, one or more pulses of UV light energy (e.g., approximately 365 nm) may result in cleaving a functional group from both the Z and Q pair and the X and Y pair, which may result in an unlocking both links, and alteration of both hairpin formations to a simple duplex. Alternatively, one or more pulses of both violate light energy (e.g., approximately 430 nm) and UV light energy (e.g., approximately 365 nm) may result in cleaving a functional group from both the Z and Q pair and the X and Y pair, which may result in an unlocking both links, and alteration of both hairpin formations to a simple duplex. These three different DNA conformations (small and large hairpin, simple duplex) may be distinguishable by nanopore sequencing platforms via differences in current flow as the structures pass through the pore (see A. T. Guy, T. J. Piggot, and S. Khalid, Biophys J. 2012; 103:1028-36, the disclosure of which is incorporated herein by reference). Accordingly, in some embodiments, the three conformations are utilized to distinguish an unwritten data bit and a binary code of two distinguishable written bits of data. And in some embodiments, the three conformations are utilized to distinguish three bits of data in a ternary code.

### Methods of Data Writing and Reading

Several embodiments are directed towards writing and reading data on polymers utilizing sets of chemically modifiable structures (e.g., fluorophores). In many embodiments, a writable polymer is provided having chemically modifiable structures installed thereupon. In some embodiments, charged groups are iteratively spaced along the polymer. The provided writable polymer may also have spacers, delimiters, and data tags, as described herein. To write data upon a polymer, in accordance with various embodiments, an individual strand is passed through a device having a nanopore. Various nanopore devices can enable the structural changes of the chemically modifiable structures as they pass through the pore (see, e.g., D. Garoli, et al., Nano Lett. 2019; 19:7553-7562; and M. Rahman, et al., Lab Chip. 2021; 21:3030-3052; the disclosures of which are each incorporated herein by reference). Photoexcitation of photocages or cleavable linkers progressively along the strand may result in breaking chemical bonds to yield a binary code of structural modification bits. Such devices may include structures near the nanopore that amplify light energy or redox energy to highly localized positions. Examples include (but are not limited to) plasmonic amplifiers such as metallic bowties, metallic nanorods, or zero mode waveguides (see, e.g., J. D. Spitzberg, et al., Adv Mater. 2019; 31:e1900422, the disclosure of which is incorporated herein by reference). Pulsing energy on such plasmonic structures can yield resolution of as small as one nanometer. Alternatively, the use of two lasers can be employed via the STED technique to achieve highly localized illumination (see, e.g., S. J. Sahl and S. W. Hell High-Resolution 3D Light Microscopy with STED and RESOLFT. 2019 Aug. 14. In: Bille J F, editor. High Resolution Imaging in Microscopy and Ophthalmology: New Frontiers in Biomedical Optics. Chain (CH): Springer; 2019. Chapter 1, the disclosure of which is incorporated herein by reference).

In some embodiments, modification of chemical modifiable structures may not be required to be performed at every repeating unit of the polymer. Units may be skipped during the writing process. When reading polymers with skipped units, such skipped units can be interpreted as blank or null and the reader progresses to the next fluorescent position to find the next bit in the string. Skipping of a repeating unit during writing and reading may be performed intentionally to space out bit writing to best suit the resolution of the writing method; skipping may also occur in random fashion due to stochastic movements and alteration of the rate of the polymeric molecule passing through the pore.

In many embodiments, the writing device is provided a software code for writing the data into the polymer. Accordingly, the writing device, directed by this code, will control pulses of energy by time and/or wavelength to selectively modify the chemically modifiable structures of the polymer to yield a data code (e.g., binary code). In some embodiments, after writing a code of data into the polymer, the polymer can be stored in the dark and by any appropriate means for maintaining integrity of the polymeric molecule. For instance, data written polymers can be stored dry, as a precipitate, or in an appropriate solution at room temperature, or at colder temperatures (e.g., −20° C.). Stabilizers such as (for example) alcohol, antioxidants, chelating agents and biological inhibitors (e.g., nuclease inhibitors or protease inhibitors), may be included with the stored polymer.

Polymers most efficiently store data at the single molecule level, providing the highest potential density of information. In some embodiments, however, if redundancy of data is required for better accuracy of data storage, then a plurality of polymers could be used to redundantly write the same data on each polymer of the plurality. Error correction algorithms are already well developed for digital data storage, and some of these algorithms can be applied in the present approach (see J. Li, et al., IEEE Transactions on Emerging Topics in Computing. 2021; 9:651-663, the disclosure of which is incorporated herein by reference).

Highly localized light excitation can be achieved via specialized sub-wavelength microscopic focusing strategies such as STEDX, or by the use of nanoplasmonic structures such as bow ties or by the use of zero-mode waveguides (see Y. Fang and M Sun, Light Sci Appl. 2015; 4:e294; and X. Shi, et al. Small. 2018; 14:e1703307; the disclosures of which are each incorporated herein by reference). In some embodiments, other methods of high-resolution optical excitation are also possible. Timing of energy pulses and controlled passage of the writable polymer can be in concert with appropriate spacing such that data is encoded with fidelity.

In many embodiments, to read the data on written nucleic acid polymers, any appropriate nanopore capable of reading fluorescence of single fluorophores or capable of analyzing structural differences can be utilized. In certain embodiments, a device is capable both of writing and reading biological or organic polymers. In certain embodiments, a single nanopore has dual functionality for both writing and reading polymers, however, some devices may include distinct nanopores for performing writing and reading. Various nanopore devices are available commercially, or alternatively, a nanopore device can be fabricated or manufactured for writing and/or reading the data. The nanopore can be comprised of solid-state materials.

In addition to utilizing a nanopore device for reading, a polymer may be stretched and imaged utilizing a device capable or reading fluorophores along stretched polymers. Polymer (especially DNA) stretching or combing methods are known to practitioners of the art (see, e.g., Z. E. Nazari and L. Gurevich, J. Self-Assembly and Molecular Electronics 2013; 1:125-148; A. Kaykov, et al., Sci Rep. 2016; 6:19636; the disclosures of which are incorporated herein by reference). Alternatively, in some embodiments, super resolution microscopy can be employed (via the STED approach or other known methods) for achieving high spatial resolution. Imaging of a single DNA strand by STED can yield a sequence of dye colors, which represents a string of bits. The method can be automated for high throughput, with many strands stretched in one field of view, and automated imaging software that reads a string of dyes (bits) and converts it into digital information.

The use of energy pulses to convert or otherwise alter dyes and/or quenchers in a repeating polymer may result in detectable structural changes in the repeating units of the polymers. Accordingly, in addition to the above fluorescence methods, written polymers can also be read by non-fluorescence methods. An example includes nanopore sequencing by the use of alterations of ion flow. Another method involves the reading of optical absorption or other spectral signatures in the altered dyes (such as vibrational modes) as the strand passes though the pore. Another method involves the reading of redox signals as the strand passes through the pore.

### NUMBERED EMBODIMENTS

Embodiment 1. A writable polymer for data encoding utilizing installed chemically modifiable structures, comprising:
a plurality of sets of one or more chemically modifiable structures iteratively spaced along a polymer, each set of one or more chemically modifiable structures attached onto a monomer that is linked to a backbone, wherein the chemically modifiable structures are capable of being modified by pulses of light energy or of redox energy.

Embodiment 2. The polymer of embodiment 1, wherein each set of one or more chemically modifiable structures comprises two caged fluorophores capable of being uncaged by the pulses of light energy or of redox energy.

Embodiment 3. The polymer of embodiment 1, wherein each set of one or more chemically modifiable structures comprises a quencher, a first fluorophore, and a caged fluorophore, wherein the quencher is capable of being released by the pulses of light energy or of redox energy, and wherein the caged fluorophore is capable of being uncaged by the pulses of light energy or of redox energy.

Embodiment 4. The polymer of any one of embodiments 1, 2 or 3, wherein each set of one or more chemically modifiable structures comprises a first fluorophore and second fluorophore within Forster resonant energy transfer distance.

Embodiment 5. The polymer of embodiment 1, wherein each set of one or more chemically modifiable structures comprises a photoconvertible fluorophore, wherein the photoconvertible fluorophore exist in a first structural state having a first emission wavelength and are capable of being converted into a second structural state having a second emission wavelength via the pulses of light energy or of redox energy.

Embodiment 6. The polymer of embodiment 5, wherein the conversion of the photoconvertible fluorophore from the first structural state into a second structural state is via the pulses of light at first wavelength; and wherein the photoconvertible fluorophore is capable of being further converted into a third structural state having a third emission wavelength via the pulses of light at a second wavelength.

Embodiment 7. The polymer of any one of embodiments 1-6 further comprising a plurality of charged constituents iteratively spaced along the polymer.

Embodiment 8. The polymer of embodiment 7, wherein the plurality of charged constituents is within the backbone.

Embodiment 9. The polymer of embodiment 7, wherein the plurality of charged constituents is within monomers of the polymer.

Embodiment 10. The polymer of embodiment 7, wherein the plurality of charged constituents is negatively charged phosphate groups, negatively charged sulfate groups, negatively charged carboxylate groups, or positively charged ammonium groups.

Embodiment 11. The polymer of any one of embodiments 1-10, wherein the polymer is a biological polymer.

Embodiment 12. The polymer of embodiment 11, wherein the polymer is DNA, RNA, phosphorothiate DNA, glycerol nucleic acids (GNA), threose nucleic acids (TNA), 2′-fluoro-DNA 2′-O-methyl RNA, locked nucleic acids (LNA), a peptide chain, or a peptoid chain.

Embodiment 13. The polymer of any one of embodiments 1-12, wherein the polymer is organic.

Embodiment 14. A writable polymer (e.g., blank tape) for data encoding utilizing secondary structure conformations, comprising:
a nucleic acid polymer comprising a plurality of strings of residues iteratively spaced along the nucleic acid polymer, wherein each string of residues of the plurality of strings of residues comprises a hairpin structure, wherein a first residue within the hairpin structure comprises a first reactive group and a second residue within the hairpin structure comprises a second reactive group, wherein the first residue and the second residue are not neighboring residues, and wherein the first reactive group is bonded with the second reactive group.

Embodiment 15. The polymer of embodiment 14, wherein the first reactive group comprises a cleavable linker.

Embodiment 16. The polymer of embodiment 14 or 15, wherein the nucleic acid polymer is single stranded.

Embodiment 17. The polymer of embodiment 14 or 15, wherein the nucleic acid polymer is double stranded, and wherein the bond between the first reactive group and the second reactive group destabilizes formation of simple duplexes between residues of the hairpin structure and residues complementing the hairpin structure.

Embodiment 18. The polymer of embodiment 17, wherein cleavage of the cleavable linker destabilizes the hairpin structure and promotes formation of simple duplexes between the residues of the hairpin structure and the residues complementing the hairpin structure.

Embodiment 19. The polymer of any one of embodiments 14-18, wherein each string of residues of the plurality of strings of residues is between 20 and 200 residues.

Embodiment 20. The polymer of any one of embodiments 14-19, wherein each string of residues of the plurality of strings of residues has the same number of residues.

Embodiment 21. The polymer of any one of embodiments 14-20, wherein each string of residues of the plurality of strings of residues has the same sequence.

Embodiment 22. A writable polymer for data encoding utilizing secondary structure conformations, comprising:
a nucleic acid polymer comprising a plurality of strings of residues iteratively spaced along the nucleic acid polymer, wherein each string of residues of the plurality of strings of residues comprises a first hairpin structure and a second hairpin structure,
wherein a first residue within the first hairpin structure comprises a first reactive group and a second residue within the first hairpin structure comprises a second reactive group, wherein the first residue and the second residue of the first hairpin structure are not neighboring residues, wherein the first reactive group of the first hairpin structure is bonded with the second reactive group of the first hairpin structure,
wherein a first residue within the second hairpin structure comprises a first reactive group and a second residue within the second hairpin structure comprises a second reactive group, wherein the first residue and the second residue of the second hairpin structure are not neighboring residues, and wherein the first reactive group of the second hairpin structure is bonded with the second reactive group of the second hairpin structure.

Embodiment 23. The polymer of embodiment 22, wherein the first reactive group of the first hairpin structure and the first reactive group of the second hairpin structure each comprise a cleavable linker, wherein the cleavable linker of the first hairpin structure is cleavable by one or more pulses of light at first wavelength, wherein the cleavable linker of the second hairpin structure is cleavable by one or more pulses of light at second wavelength, and wherein the cleavable linker of the second hairpin structure is not cleavable by the one or more pulses of light at the first wavelength.

Embodiment 24. The polymer of embodiment 23, wherein the cleavable linker of the first hairpin structure is also cleavable by the one or more pulses of light at the second wavelength.

Embodiment 25. The polymer of embodiment 23, wherein the cleavable linker of the first hairpin structure is not cleavable by the one or more pulses of light at the second wavelength.

Embodiment 26. The polymer of any one of embodiments 22-25, wherein the nucleic acid polymer is single stranded.

Embodiment 27. The polymer of any one of embodiments 22-25, wherein the nucleic acid polymer is double stranded, and wherein the bond between the first reactive group and the second reactive group of the first hairpin structure destabilizes formation of simple duplexes between residues of the first hairpin structure and residues complementing the first hairpin structure.

Embodiment 28. The polymer of embodiment 27, wherein cleavage of the cleavable linker of the first hairpin structure destabilizes the first hairpin structure and promotes formation of simple duplexes between the residues of the first hairpin structure and the residues complementing the first hairpin structure.

Embodiment 29. The polymer of any one of embodiments 22-28, wherein the nucleic acid polymer is double stranded, and wherein the bond between the first reactive group and the second reactive group of the second hairpin structure destabilizes formation of simple duplexes between residues of the second hairpin structure and residues complementing the second hairpin structure.

Embodiment 30. The polymer of embodiment 29, wherein cleavage of the cleavable linker of the second hairpin structure destabilizes the second hairpin structure and promotes formation of simple duplexes between the residues of the second hairpin structure and the residues complementing the second hairpin structure.

Embodiment 31. The polymer of any one of embodiments 22-30, wherein the number of residues of the first hairpin structure is not equal to the number of residues of the second hairpin structure.

Embodiment 32. The polymer of any one of embodiments 22-31, wherein each string of residues of the plurality of strings of residues is between 20 and 200 residues.

Embodiment 33. The polymer of any one of embodiments 22-32, wherein each string of residues of the plurality of strings of residues has the same number of residues.

Embodiment 34. The polymer of any one of embodiments 22-33, wherein each string of residues of the plurality of strings of residues has the same sequence.

Embodiment 35. A polymer encoded with data utilizing installed chemically modifiable structures, comprising:
a plurality of sets of one or more chemically modifiable structures iteratively spaced along a polymer, each set of one or more chemically modifiable structures attached onto a monomer that is linked to a backbone, wherein the plurality of sets of one or more chemically modifiable structures comprises a plurality of modified structures that have been modified from a first structural state into a second structural state via pulses of light or redox energy.

Embodiment 36. The polymer of embodiment 35, wherein each set of one or more chemically modifiable structures of the plurality of sets of one or more chemically modifiable structures was installed onto its monomer in the first structural state; and wherein the plurality of modified structures was modified via device comprising a nanopore, the device is capable of traversing the polymer through the nanopore and selectively modifying the plurality of sets of one or more chemically modifiable structures via the pulses of light or redox energy in accordance with a data code.

Embodiment 37. The polymer of embodiment 35 or 36, wherein the plurality of modified structures comprises uncaged fluorophores that have been uncaged by the pulses of light or redox energy.

Embodiment 38. The polymer of embodiment 35, 36, or 37, wherein the plurality of modified structures comprises structures with quenchers that have been released by the pulses of light or redox energy.

Embodiment 39. The polymer of embodiment 35 or 36, wherein the plurality of modified structures comprises photoconverted fluorophores, wherein the second structural state of the photoconverted fluorophores emits fluorescence at wavelength that is different than the wavelength emitted by the first structural state, and wherein photoconverted fluorophore was converted by the pulses of light energy.

Embodiment 40. A polymer encoded with data utilizing secondary structure conformations, comprising:
a nucleic acid polymer comprising a plurality of strings of residues iteratively spaced structures iteratively spaced along a polymer, wherein the plurality of sets of strings of residues comprises a plurality of modified structures that have been modified from a first structural hairpin state into a second structural simple duplex state via pulses of light or redox energy.

Embodiment 41. The polymer of embodiment 40, wherein the plurality of sets of strings of residues further comprises a plurality of unmodified structures, wherein each structure of the plurality unmodified structures is a hairpin structure, wherein a first residue within the hairpin structure comprises a first reactive group and a second residue within the hairpin structure comprises a second reactive group, wherein the first residue and the second residue are not neighboring residues, and wherein the first reactive group is bonded with the second reactive group.

Embodiment 42. The polymer of embodiment 41, wherein the plurality of modified structures and the plurality of unmodified structures are a digital code.

Embodiment 43. The polymer of embodiment 41 or 42, wherein the nucleic acid polymer is double stranded, and wherein the bond between the first reactive group and the second reactive group destabilizes formation of simple duplexes between residues of the hairpin structure and residues complementing the hairpin structure.

Embodiment 44. The polymer of embodiment 41, wherein the plurality of modified structures was modified via device comprising a nanopore, the device is capable of traversing the polymer through the nanopore and selectively modifying the first structural hairpin state of the plurality of strings of residues via the pulses of light or redox energy in accordance with a data code.

Embodiment 45. The polymer of embodiment 41 or 44, wherein the plurality of modified structures was modified by cleaving a linker stabilizing the first structural hairpin state, wherein cleaving the linker allowed the modification from the first structural hairpin state into the second structural simple duplex, and wherein the linker was cleaved via the pulses of light or redox energy.

Embodiment 46. The polymer of any one of embodiments 41-45, wherein each string of residues of the plurality of strings of residues is between 20 and 200 residues.

Embodiment 47. The polymer of any one of embodiments 41-46, wherein each string of residues of the plurality of strings of residues has the same number of residues.

Embodiment 48. The polymer of any one of embodiments 41-47, wherein each string of residues of the plurality of strings of residues has the same sequence.

Embodiment 49. A method of encoding data onto a writable polymer, comprising:
providing a writable polymer that comprises a plurality of sets of one or more chemically modifiable structures iteratively spaced along and linked via the polymer backbone, wherein each set of one or more chemically modifiable structures of the plurality of sets of one or more chemically modifiable structures is provided having a first state and is capable of being modified from the first structural state into a second structural state;
selectively modifying, utilizing a data writing device, a subset of the plurality of sets of one or more chemically modifiable structures into the second structural state such that a data encoded polymer is generated.

Embodiment 50. The method of embodiment 49, wherein the data writing device comprise a nanopore, and the method further comprising:
passing the writable polymer through the nanopore of the writing device, wherein the nanopore comprises a means to impinge pulses of light energy or redox energy onto the subset of the plurality of sets of one or more chemically modifiable structures into the second state.

Embodiment 51. The method of embodiment 49 or 50, wherein the plurality of sets of one or more chemically modifiable structures comprises caged fluorophores that are uncaged via the pulses of light energy or redox energy.

Embodiment 52. The method of embodiment 49, 50 or 51, wherein the plurality of sets of one or more chemically modifiable structures comprises fluorophores in proximity of a quencher, wherein the quencher is released the via the pulses of light energy or redox energy.

Embodiment 53. The method of embodiment 49 or 50, wherein the plurality of sets of one or more chemically modifiable structures comprises photoconvertible fluorophores, wherein the first structural state of the photoconvertible fluorophores has a first emission wavelength and the second structural state of the photoconvertible fluorophores has a second emission wavelength.

Embodiment 54. The method of any one of embodiments 49-53, further comprising:
reading the encoded data of the generated polymer.

Embodiment 55. The method of embodiment 54, wherein the encoded data is read by passing the generated polymer through a nanopore of a data reading device, wherein the nanopore of the reading device comprises a means to detect fluorescence of the second state of the plurality of sets of one or more chemically modifiable structures.

Embodiment 56. The method of embodiment 55, wherein the data reading device is the same device as the data writing device.

Embodiment 57. The method of embodiment 56, wherein the nanopore device to read data is the same nanopore device to write data.

Embodiment 58. The method of embodiment 54, wherein the encoded data is read by stretching and imaging the generated polymer.

Embodiment 59. A method of encoding data onto a writable polymer, comprising:
providing a writable nucleic acid polymer that comprises a plurality of strings of residues iteratively spaced along the writable nucleic acid polymer, wherein each string of residues of the plurality of strings of residues comprises a hairpin structure, wherein a first residue within the hairpin structure comprises a first reactive group and a second residue within the hairpin structure comprises a second reactive group, wherein the first residue and the second residue are not neighboring residues, and wherein the first reactive group is bonded with the second reactive group;
selectively modifying, utilizing a data writing device, the hairpin structure of a subset of the plurality of strings of residues into a simple duplex structural state.

Embodiment 60. The method of embodiment 59, wherein the data writing device comprise a nanopore, and the method further comprising:
passing the writable nucleic acid polymer through the nanopore of the writing device, wherein the nanopore comprises a means to impinge pulses of light energy or redox energy onto the hairpin structure of the subset of the plurality of strings of residues such that each hairpin structure of the subset of the plurality of strings of residues is modified into the simple duplex structural state.

Embodiment 61. The method of embodiment 60, wherein the first reactive group comprises a cleavable linker, and wherein the impinged pulses of light energy or redox energy cleaves the cleavable linker the first reactive group of the hairpin structure of the subset of the plurality of strings of residues.

Embodiment 62. The method of embodiment 61, wherein cleavage of the cleavable linker destabilizes the hairpin structure and promotes formation of simple duplexes between residues of the hairpin structure and residues complementing the hairpin structure.

Embodiment 63. The polymer of any one of embodiments 59-62, wherein each string of residues of the plurality of strings of residues is between 20 and 200 residues.

Embodiment 64. The polymer of any one of embodiments 59-63, wherein each string of residues of the plurality of strings of residues has the same number of residues.

Embodiment 65. The polymer of any one of embodiments 59-64, wherein each string of residues of the plurality of strings of residues has the same sequence.

Embodiment 66. A method of encoding data onto a writable polymer, comprising:
providing a nucleic acid polymer comprising a plurality of strings of residues iteratively spaced along the nucleic acid polymer, wherein each string of residues of the plurality of strings of residues comprises a first hairpin structure and a second hairpin structure,
wherein a first residue within the first hairpin structure comprises a first reactive group and a second residue within the first hairpin structure comprises a second reactive group, wherein the first residue and the second residue of the first hairpin structure are not neighboring residues, wherein the first reactive group of the first hairpin structure is bonded with the second reactive group of the first hairpin structure,
wherein a first residue within the second hairpin structure comprises a first reactive group and a second residue within the second hairpin structure comprises a second reactive group, wherein the first residue and the second residue of the second hairpin structure are not neighboring residues, and wherein the first reactive group of the second hairpin structure is bonded with the second reactive group of the second hairpin structure;
selectively modifying, utilizing pulses of light energy at first wavelength, only the first hairpin structure of a first subset of the plurality of strings of residues into a simple duplex structural state; and
selectively modifying, utilizing pulses of light energy at a second wavelength, the second hairpin structure of a second subset of the plurality of strings of residues into a simple duplex structural state.

Embodiment 67. The method of embodiment 66, wherein the first reactive group of the first hairpin comprises a first cleavable linker, and wherein the pulses of light energy at the first wavelength cleaves the first cleavable linker the first reactive group of the first hairpin structure of the first subset of the plurality of strings of residues.

Embodiment 68. The method of embodiment 66, wherein the first reactive group of the second hairpin comprises a second cleavable linker, and wherein the pulses of light energy at the second wavelength cleaves the second cleavable linker the first reactive group of the second hairpin structure of the second subset of the plurality of strings of residues.

Embodiment 69. A method for installing sets of chemically modifiable structures onto a nucleic acid, the method comprising:
providing a nucleic acid polymer in a solution;
adding to the solution sets of chemically modifiable structures, wherein each set of chemically modifiable structures is functionalized with a chemical group capable of reacting with a nucleobase of the nucleic acid polymer such that a writable polymer with iteratively spaced sets of chemically modifiable structures is yielded, and wherein the chemically modifiable structures are capable of being modified by pulses of light energy or of redox energy.

Embodiment 70. The method of embodiment 69, wherein the chemical group is a leaving group attached to an sp3 carbon, and wherein the nucleobase has an available amine; or wherein the chemical group is an amine, and wherein the nucleobase has an available leaving group attached to an sp3 carbon.

Embodiment 71. The method of embodiment 70, wherein the leaving group is iodide, bromide, chloride, tosylate, triflate, or mesylate.

Embodiment 72. The method of embodiment 69, wherein the chemical group is an amine, wherein the nucleobase has an available carboxylic acid, and wherein a condensing or a coupling reagent is utilized to promote a reaction between the chemical group and the nucleobase; or
wherein the chemical group is a carboxylic acid, wherein the nucleobase has an available amine, and wherein a condensing or a coupling reagent is utilized to promote a reaction between the chemical group and the nucleobase.

Embodiment 73. The method of embodiment 69, wherein the chemical group is an amine, and wherein the nucleobase has an available activated ester; or wherein the chemical group is an activated ester, and wherein the nucleobase has an available amine.

Embodiment 74. The method of embodiment 69, wherein the chemical group is an amine, and wherein the nucleobase has an available isocyanate or an available isothiocyanate; or wherein the chemical group is an isocyanate or an isothiocyanate, and wherein the nucleobase has an available amine.

Embodiment 75. The method of embodiment 69, wherein the chemical group is an azide, and wherein the nucleobase has an available alkyne; or wherein the chemical group is an alkyne, and wherein the nucleobase has an available azide.

Embodiment 76. The method of embodiment 69, wherein the chemical group is a tetrazine, and wherein the nucleobase has an available strained alkene; or wherein the chemical group is a strained alkene, and wherein the nucleobase has an available tetrazine.

Embodiment 77. The method of embodiment 69, wherein the chemical group is an aldehyde or a ketone, and wherein the nucleobase has an available aminooxy group or an available hydrazine group; or wherein the chemical group is an aminooxy group or a hydrazine group, and wherein the nucleobase has an available aldehyde or an available ketone.

Embodiment 78. The method of embodiment 69, wherein the chemical group is thiol, and wherein the nucleobase has an available Michael acceptor; or wherein the chemical group is a Michael acceptor, and wherein the nucleobase has an available thiol.

Embodiment 79. The method of embodiment 78, wherein the Michael acceptor is a maleimide or a bromomaleimide.

Embodiment 80. The method of embodiment 69, wherein the chemical group is thiol, and wherein the nucleobase has an available disulfide; or wherein the chemical group is a disulfide, and wherein the nucleobase has an available thiol.

Embodiment 81. The method of embodiment 69, wherein the chemical group is a thiol, wherein the nucleobase has an available alkene, and wherein light is utilized to promote a reaction between the chemical group and the nucleobase; or wherein the chemical group is an alkene, wherein the nucleobase has an available thiol, and wherein light is utilized to promote a reaction between the chemical group and the nucleobase.

Embodiment 82. The method of embodiment 69, wherein the chemical group is an amine, a thiol, or an alcohol, and wherein the nucleobase has an available activated sulfonyl species; or wherein the chemical group is an activated sulfonyl species, and wherein the nucleobase has an available amine, an available thiol, or an available alcohol.

Embodiment 83. The method of embodiment 78, wherein the activated sulfonyl species is a sulfur(VI) exchange substrate, a sulfuryl fluoride, or an iminosulfur oxydifluoride.

Embodiment 84. The method of embodiment 69, wherein the chemical group is an alkene, wherein the nucleobase has an available alkene, and wherein a metal is utilized to promote a reaction between the chemical group and the available alkene on the nucleobase.

Embodiment 85. The method of embodiment 69, wherein the chemical group is an amine, a thiol, or an alcohol, and wherein the nucleobase has an available activated aryl group; or
wherein the chemical group is an activated aryl group, and wherein the nucleobase has an available amine, an available thiol, or an available alcohol.

Embodiment 86. The method of embodiment 85, wherein the activated aryl species is a pentafluorobenzene or a para-chloro-acylbenzene.

Embodiment 87. The method of any one of embodiments 69-86, wherein each set of one or more chemically modifiable structures comprises two caged fluorophores capable of being uncaged by the pulses of light energy or of redox energy.

Embodiment 88. The method of any one of embodiments 69-86, wherein each set of one or more chemically modifiable structures comprises a quencher, a first fluorophore, and a caged fluorophore, wherein the quencher is capable of being released by the pulses of light energy or of redox energy, and wherein the caged fluorophore is capable of being uncaged by the pulses of light energy or of redox energy.

Embodiment 89. The method of any one of embodiments 69-88, wherein each set of one or more chemically modifiable structures comprises a first fluorophore and second fluorophore within Forster resonant energy transfer distance.

Embodiment 90. The method of any one of embodiments 69-86, wherein each set of one or more chemically modifiable structures comprises a photoconvertible fluorophore, wherein the photoconvertible fluorophore exist in a first structural state having a first emission wavelength and are capable of being converted into a second structural state having a second emission wavelength via the pulses of light energy or of redox energy.

Embodiment 91. The method of embodiment 90, wherein the conversion of the photoconvertible fluorophore from the first structural state into a second structural state is via the pulses of light at first wavelength; and wherein the photoconvertible fluorophore is capable of being converted into a third structural state having a third emission wavelength via the pulses of light at a second wavelength.

Embodiment 92. A method for installing sets of chemically modifiable structures onto nucleic acid, the method comprising:
providing a single-stranded nucleic acid polymer in a first solution;
adding to the first solution a primer sequence complementary to the single-stranded nucleic acid polymer, a set of deoxynucleotides tri-phosphates, and a nucleic acid polymerase, wherein the set of deoxynucleotides tri-phosphates comprises modified deoxynucleotides tri-phosphates that have been modified with an available functional group;
allowing the nucleic acid polymerase to polymerize a complement strand of the single-stranded nucleic acid polymer, wherein the modified deoxynucleotides are incorporated into the complement strand, yielding the complement strand incorporated with modified nucleobases having the available functional group;
incubating the complement strand with sets of chemically modifiable structures in a second solution, wherein each set of chemically modifiable structures is functionalized with a chemical group capable of reacting with the available functional group of the modified nucleobases incorporated into the complement strand such that a writable polymer with iteratively spaced sets of chemically modifiable structures is yielded, and wherein the chemically modifiable structures are capable of being modified by pulses of light energy or of redox energy.

Embodiment 93. The method of embodiment 92, wherein the chemical group is a leaving group attached to an sp3 carbon, and wherein the available functional group is an amine; or wherein the chemical group is an amine, and wherein the available functional group is a leaving group attached to an sp3 carbon.

Embodiment 94. The method of embodiment 93, wherein the leaving group is iodide, bromide, chloride, tosylate, triflate, or mesylate.

Embodiment 95. The method of embodiment 92, wherein the chemical group is an amine, wherein the available functional group is a carboxylic acid, and wherein a condensing or a coupling reagent is utilized to promote a reaction between the chemical group and the available functional group; or wherein the chemical group is a carboxylic acid, wherein the available functional group is an amine, and wherein a condensing or a coupling reagent is utilized to promote a reaction between the chemical group and the available functional group.

Embodiment 96. The method of embodiment 92, wherein the chemical group is an amine, and wherein the available functional group is an activated ester; or wherein the chemical group is an activated ester, and wherein the available functional group is an amine.

Embodiment 97. The method of embodiment 92, wherein the chemical group is an amine, and wherein the available functional group is an isocyanate or an isothiocyanate; or wherein the chemical group is an isocyanate or an isothiocyanate, and wherein the available functional group is an amine.

Embodiment 98. The method of embodiment 92, wherein the chemical group is an azide, and wherein the available functional group is an alkyne; or wherein the chemical group is an alkyne, and wherein the available functional group is an azide.

Embodiment 99. The method of embodiment 92, wherein the chemical group is a tetrazine, and wherein the available functional group is a strained alkene; or wherein the chemical group is a strained alkene, and wherein the available functional group is a tetrazine.

Embodiment 100. The method of embodiment 92, wherein the chemical group is an aldehyde or a ketone, and wherein the available functional group is an aminooxy group or an hydrazine group; or wherein the chemical group is an aminooxy group or a hydrazine group, and wherein the available functional group is an aldehyde or a ketone.

Embodiment 101. The method of embodiment 92, wherein the chemical group is thiol, and wherein the available functional group is a Michael acceptor; or wherein the chemical group is a Michael acceptor, and wherein the available functional group is a thiol.

Embodiment 102. The method of embodiment 101, wherein the Michael acceptor is a maleimide or a bromomaleimide.

Embodiment 103. The method of embodiment 92, wherein the chemical group is thiol, and wherein the available functional group is a disulfide; or wherein the chemical group is a disulfide, and wherein the available functional group is a thiol.

Embodiment 104. The method of embodiment 92, wherein the chemical group is a thiol, wherein the available functional group is an alkene, and wherein light is utilized to promote a reaction between the chemical group and the available alkene on the nucleobase; or wherein the chemical group is an alkene, wherein the available functional group is a thiol, and wherein light is utilized to promote a reaction between the chemical group and the available functional group.

Embodiment 105. The method of embodiment 92, wherein the chemical group is an amine, a thiol, or an alcohol, and wherein the available functional group is an activated sulfonyl species; or wherein the chemical group is an activated sulfonyl species, and wherein the available functional group is an amine, a thiol, or an alcohol.

Embodiment 106. The method of embodiment 105, wherein the activated sulfonyl species is a sulfur(VI) exchange substrate, a sulfuryl fluoride, or an iminosulfur oxydifluoride.

Embodiment 107. The method of embodiment 92, wherein the chemical group is an alkene, wherein the available functional group is an alkene, and wherein a metal is utilized to promote a reaction between the chemical group and the available functional group.

Embodiment 108. The method of embodiment 92, wherein the chemical group is an amine, a thiol, or an alcohol, and wherein the available functional group is an activated aryl group; or
wherein the chemical group is an activated aryl group, and wherein the available functional group is an amine, a thiol, or an alcohol.

Embodiment 109. The method of embodiment 108, wherein the activated aryl species is a pentafluorobenzene or a para-chloro-acylbenzene.

Embodiment 110. The method of any one of embodiments 92-109, wherein each set of one or more chemically modifiable structures comprises two caged fluorophores capable of being uncaged by the pulses of light energy or of redox energy.

Embodiment 111. The method of any one of embodiments 92-109, wherein each set of one or more chemically modifiable structures comprises a quencher, a first fluorophore, and a caged fluorophore, wherein the quencher is capable of being released by the pulses of light energy or of redox energy, and wherein the caged fluorophore is capable of being uncaged by the pulses of light energy or of redox energy.

Embodiment 112. The method of any one of embodiments 92-111, wherein each set of one or more chemically modifiable structures comprises a first fluorophore and second fluorophore within Forster resonant energy transfer distance.

Embodiment 113. The method of any one of embodiments 92-109, wherein each set of one or more chemically modifiable structures comprises a photoconvertible fluorophore, wherein the photoconvertible fluorophore exist in a first structural state having a first emission wavelength and are capable of being converted into a second structural state having a second emission wavelength via the pulses of light energy or of redox energy.

Embodiment 114. The method of embodiment 113, wherein the conversion of the photoconvertible fluorophore from the first structural state into a second structural state is via the pulses of light at first wavelength; and wherein the photoconvertible fluorophore is capable of being converted into a third structural state having a third emission wavelength via the pulses of light at a second wavelength.

Embodiment 115. A method for installing sets of chemically modifiable structures onto an organic polymer, the method comprising:
providing an organic polymer in a solution, wherein the organic polymer contains a first available functional group on a plurality of its monomers;
adding to the solution sets of chemically modifiable structures, wherein each set of chemically modifiable structures is functionalized with a second available functional group capable of reacting with the plurality of the first available functional groups on the organic polymer such that a writable polymer with iteratively spaced sets of chemically modifiable structures is yielded, and wherein the chemically modifiable structures are capable of being modified by pulses of light energy or of redox energy.

Embodiment 116. The method of embodiment 115, wherein the first available functional group is a leaving group attached to an sp3 carbon, and wherein the second available functional group is an amine; or wherein the first available functional group is an amine, and wherein the second available functional group is a leaving group attached to an sp3 carbon.

Embodiment 117. The method of embodiment 116, wherein the leaving group is iodide, bromide, chloride, tosylate, triflate, or mesylate.

Embodiment 118. The method of embodiment 115, wherein the first available functional group is an amine, wherein the second available functional group is a carboxylic acid, and wherein a condensing or a coupling reagent is utilized to promote a reaction between the first available functional group and the second available functional group; or wherein the first available functional group is a carboxylic acid, wherein the second available functional group is an amine, and wherein a condensing or a coupling reagent is utilized to promote a reaction between the first available functional group and the second available functional group.

Embodiment 119. The method of embodiment 115, wherein the first available functional group is an amine, and wherein the second available functional group is an activated ester; or
wherein the first available functional group is an activated ester, and wherein the second available functional group is an amine.

Embodiment 120. The method of embodiment 115, wherein the first available functional group is an amine, and wherein the second available functional group is an isocyanate or an isothiocyanate; or
wherein the first available functional group is an isocyanate or an isothiocyanate, and wherein the second available functional group is an amine.

Embodiment 121. The method of embodiment 115, wherein the first available functional group is an azide, and wherein the second available functional group is an alkyne; or
wherein the first available functional group is an alkyne, and wherein the second available functional group is an azide.

Embodiment 122. The method of embodiment 115, wherein the first available functional group is a tetrazine, and wherein the second available functional group is a strained alkene; or
wherein the first available functional group is a strained alkene, and wherein the second available functional group is a tetrazine.

Embodiment 123. The method of embodiment 115, wherein the first available functional group is an aldehyde or a ketone, and wherein the second available functional group is an aminooxy group or an hydrazine group; or
wherein the first available functional group is an aminooxy group or a hydrazine group, and
wherein the second available functional group is an aldehyde or a ketone.

Embodiment 124. The method of embodiment 115, wherein the first available functional group is thiol, and wherein the second available functional group is a Michael acceptor; or wherein the first available functional group is a Michael acceptor, and wherein the second available functional group is a thiol.

Embodiment 125. The method of embodiment 124, wherein the Michael acceptor is a maleimide or a bromomaleimide.

Embodiment 126. The method of embodiment 115, wherein the first available functional group is thiol, and wherein the second available functional group is a disulfide; or wherein the first available functional group is a disulfide, and wherein the second available functional group is a thiol.

Embodiment 127. The method of embodiment 115, wherein the first available functional group is a thiol, wherein the second available functional group is an alkene, and wherein light is utilized to promote a reaction between the first available functional group and the alkene on the polymer; or
wherein the first available functional group is an alkene, wherein the second available functional group is a thiol, and wherein light is utilized to promote a reaction between the first available functional group and the second available functional group.

Embodiment 128. The method of embodiment 115, wherein the first available functional group is an amine, a thiol, or an alcohol, and wherein the second available functional group is an activated sulfonyl species; or
wherein the first available functional group is an activated sulfonyl species, and wherein the second available functional group is an amine, a thiol, or an alcohol.

Embodiment 129. The method of embodiment 128, wherein the activated sulfonyl species is a sulfur(VI) exchange substrate, a sulfuryl fluoride, or an iminosulfur oxydifluoride.

Embodiment 130. The method of embodiment 115, wherein the first available functional group is an alkene, wherein the second available functional group is an alkene, and wherein a metal is utilized to promote a reaction between the first available functional group and the second available functional group.

Embodiment 131. The method of embodiment 115, wherein the first available functional group is an amine, a thiol, or an alcohol, and wherein the second available functional group is an activated aryl group; or
wherein the first available functional group is an activated aryl group, and wherein the second available functional group is an amine, a thiol, or an alcohol.

Embodiment 132. The method of embodiment 108, wherein the activated aryl species is a pentafluorobenzene or a para-chloro-acylbenzene.

Embodiment 133. The method of any one of embodiments 115-132, wherein each set of one or more chemically modifiable structures comprises two caged fluorophores capable of being uncaged by the pulses of light energy or of redox energy.

Embodiment 134. The method of any one of embodiments 115-132, wherein each set of one or more chemically modifiable structures comprises a quencher, a first fluorophore, and a caged fluorophore, wherein the quencher is capable of being released by the pulses of light energy or of redox energy, and wherein the caged fluorophore is capable of being uncaged by the pulses of light energy or of redox energy.

Embodiment 135. The method of any one of embodiments 115-134, wherein each set of one or more chemically modifiable structures comprises a first fluorophore and second fluorophore within Forster resonant energy transfer distance.

Embodiment 136. The method of any one of embodiments 115-132, wherein each set of one or more chemically modifiable structures comprises a photoconvertible fluorophore, wherein the photoconvertible fluorophore exist in a first structural state having a first emission wavelength and are capable of being converted into a second structural state having a second emission wavelength via the pulses of light energy or of redox energy.

Embodiment 137. The method of embodiment 136, wherein the conversion of the photoconvertible fluorophore from the first structural state into a second structural state is via the pulses of light at first wavelength; and wherein the photoconvertible fluorophore is capable of being converted into a third structural state having a third emission wavelength via the pulses of light at a second wavelength.

Embodiment 138. A writable polymer for data encoding, comprising:
a plurality of modifiable fluorophores iteratively spaced along the polymer and linked via a backbone, wherein the modifiable fluorophores are capable of being modified by light pulses; and
a plurality of charged constituents iteratively spaced along the polymer.

Embodiment 139. The polymer of embodiment 138, wherein the plurality of modifiable fluorophores comprises caged fluorophores capable of being uncaged via the light pulses.

Embodiment 140. The polymer of embodiment 138, wherein the plurality of modifiable fluorophores comprises fluorophores in proximity of a quencher, wherein the quencher is capable of being released the via the light pulses.

Embodiment 141. The polymer of embodiment 138, wherein the plurality of modifiable fluorophores comprises photoconvertible fluorophores, wherein the photoconvertible fluorophores exist in a first structural state having a first emission wavelength and are capable of being converted into a second structural state having a second emission wavelength via the light pulses.

Embodiment 142. The polymer of embodiment 141, wherein the conversion of the photoconvertible fluorophores from the first structural state into a second structural state is via light pulses at first wavelength; and wherein the photoconvertible fluorophores are capable of being converted into a third structural state having a third emission wavelength via light pulses at a second wavelength.

Embodiment 143. The polymer of embodiment 138, wherein the plurality of modifiable fluorophores comprises a plurality of pairs of constitutive fluorophores and caged fluorophores within Forster resonant energy transfer distance; wherein the plurality of pairs of constitutive fluorophores and caged fluorophores are iteratively spaced along the polymer; and wherein the caged fluorophores are capable of being uncaged via light pulses.

Embodiment 144. The polymer of embodiment 138, wherein the plurality of modifiable fluorophores comprises a plurality of pairs of constitutive fluorophores and quenched fluorophores within Forster resonant energy transfer distance; wherein the plurality of pairs of constitutive fluorophores and quenched fluorophores are iteratively spaced along the polymer; and wherein each quenched fluorophore is in proximity of quencher that is capable of being released via light pulses.

Embodiment 145. The polymer of embodiment 138, wherein the plurality of modifiable fluorophores comprises a plurality of pairs of two caged fluorophores within Forster resonant energy transfer distance; wherein the plurality of pairs of two caged fluorophores are iteratively spaced along the polymer; wherein each caged fluorophore of the pairs is capable of being uncaged via light pulses; and wherein a first caged fluorophore of the pairs is capable of emitting fluorescence at a first wavelength when uncaged and a second caged fluorophore of the pairs is capable of emitting fluorescence at a second wavelength when uncaged.

Embodiment 146. The polymer of embodiment 145, wherein the first caged fluorophore of the pairs is uncaged via light pulses at a first wavelength and the second caged fluorophore of the pairs is uncaged via light pulses at a second wavelength.

Embodiment 147. The polymer of embodiment 145, wherein the first caged fluorophore of the pairs is uncaged via light pulses at a first wavelength and the second caged fluorophore of the pairs is uncaged via light pulses at the first wavelength or a second wavelength.

Embodiment 148. The polymer of embodiment 138, wherein the plurality of modifiable fluorophores comprises a plurality of pairs of two quenched fluorophores within Forster resonant energy transfer distance; wherein the plurality of pairs of two quenched fluorophores are iteratively spaced along the polymer; wherein each quenched fluorophore of the pairs is in proximity of quencher that is capable of being released via light pulses; and wherein a first quenched fluorophore of the pairs is capable of emitting fluorescence at a first wavelength when its proximate quencher is released and a second quenched fluorophore of the pairs is capable of emitting fluorescence at a second wavelength when its proximate quencher is released.

Embodiment 149. The polymer of embodiment 148, wherein the first quenched fluorophore of the pairs is in proximity of a quencher that is releasable via light pulses at a first wavelength and the second quenched fluorophore of the pairs is in proximity of a quencher that is releasable via light pulses at a second wavelength.

Embodiment 150. The polymer of embodiment 148, wherein the first quenched fluorophore of the pairs is in proximity of a quencher that is releasable via light pulses at a first wavelength and the second quenched fluorophore of the pairs is in proximity of a quencher that is releasable via light pulses at the first wavelength or a second wavelength.

Embodiment 151. The polymer of embodiment 138, wherein the plurality of modifiable fluorophores comprises a plurality of pairs of fluorophores within Forster resonant energy transfer distance; wherein a first fluorophore of the pairs is caged and capable of being uncaged via light pulses; wherein a second fluorophore of the pairs is a quenched fluorophore in proximity of a quencher that is capable of being released via light pulses; wherein the plurality of pairs of the two fluorophores are iteratively spaced along the polymer; and wherein the caged fluorophore of the pairs is capable of emitting fluorescence at a first wavelength when uncaged and the quenched fluorophore of the pairs is capable of emitting fluorescence at a second wavelength when its proximate quencher is released.

Embodiment 152. The polymer of embodiment 151, wherein the first caged fluorophore of the pairs is uncaged via light pulses at a first wavelength and the second quenched fluorophore of the pairs is in proximity of a quencher that is releasable via light pulses at a second wavelength.

Embodiment 153. The polymer of embodiment 151, wherein the first caged fluorophore of the pairs is uncaged via light pulses at a first wavelength and the second quenched fluorophore of the pairs is in proximity of a quencher that is releasable via light pulses at the first wavelength or a second wavelength.

154. The polymer of embodiment 151, wherein the first caged fluorophore of the pairs is uncaged via light pulses at a first wavelength or a second wavelength and the second quenched fluorophore of the pairs is in proximity of a quencher that is releasable via light pulses at the first wavelength.

Embodiment 155. The polymer of embodiment 138, wherein the plurality of charged constituents is within the backbone.

Embodiment 156. The polymer of embodiment 138, wherein the plurality of charged constituents is within monomers of the polymer.

Embodiment 157. The polymer of embodiment 138, wherein the plurality of charged constituents is negatively charged phosphate groups, negatively charged sulfate groups, negatively charged carboxylate groups, or positively charged ammonium groups.

Embodiment 158. The polymer of embodiment 138, wherein the polymer is a biological polymer.

Embodiment 159. The polymer of embodiment 158, wherein the polymer is DNA, RNA, phosphorothiate DNA, glycerol nucleic acids (GNA), threose nucleic acids (TNA), 2′-fluoro-DNA 2′-O-methyl RNA, locked nucleic acids (LNA), a peptide chain, or a peptoid chain.

Embodiment 160. The polymer of embodiment 158, wherein the polymer is organic.

Embodiment 161. A polymer encoded with data, comprising:
a plurality of fluorophores iteratively spaced along the polymer and linked via a backbone, wherein the plurality of fluorophores comprises a plurality of modified fluorophores that have been modified from a first structural state into a second structural state via light pulses; and a plurality of charged constituents iteratively spaced along the polymer.

Embodiment 162. The polymer of embodiment 161, wherein the plurality of fluorophores was incorporated into the polymer in the first structural state; and wherein the plurality of modified fluorophores was modified via device comprising a nanopore, the device is capable of traversing the polymer through the nanopore and selectively modifying the plurality of fluorophores vial the light pulses in accordance with a data code.

Embodiment 163. The polymer of embodiment 161, wherein the plurality of modified fluorophores comprises uncaged fluorophores.

Embodiment 164. The polymer of embodiment 161, wherein the plurality of modified fluorophores comprises unquenched fluorophores.

Embodiment 165. The polymer of embodiment 161, wherein the plurality of modified fluorophores comprises photoconverted fluorophores, wherein the second structural state of the photoconverted fluorophores emits fluorescence at wavelength that is different than the wavelength emitted by the first structural state.

Embodiment 166. A method of encoding data onto a writable polymer, comprising:
providing a writable polymer that comprises a plurality of modifiable fluorophores iteratively spaced along and linked via the polymer backbone, wherein each modifiable fluorophore of the plurality of modifiable fluorophores is provided having a first state and is capable of being modified from the first structural state into a second structural state;
selectively modifying, utilizing a data writing device, a subset of the plurality of modifiable fluorophores into the second structural state such that a data encoded polymer is generated.

Embodiment 167. The method of embodiment 166, wherein the data writing device comprise a nanopore, and the method further comprising:
passing the writable nucleic acid polymer through the nanopore of the writing device, wherein the nanopore comprises a means to impinge light pulses onto the subset of the plurality of modifiable fluorophores into the second state.

Embodiment 168. The method of embodiment 166, wherein the plurality of modifiable fluorophores comprises caged fluorophores capable of being uncaged via the light pulses.

Embodiment 169. The method of embodiment 166, wherein the plurality of modifiable fluorophores comprises fluorophores in proximity of a quencher, wherein the quencher is capable of being released the via the light pulses.

Embodiment 170. The method of embodiment 166, wherein the plurality of modifiable fluorophores comprises photoconvertible fluorophores, wherein the first structural state of the photoconvertible fluorophores has a first emission wavelength and the second structural state of the photoconvertible fluorophores has a second emission wavelength.

Embodiment 171. The method of embodiment 166, further comprising:
reading the encoded data of the generated polymer.

Embodiment 172. The method of embodiment 171, wherein the encoded data is read by passing the generated polymer through the nanopore of a data reading device, wherein the nanopore of the reading device comprises a means to detect fluorescence of the second state of the plurality of modifiable fluorophores.

Embodiment 173. The method of embodiment 172, wherein the data reading device is the same device as the data writing device.

Embodiment 174. The method of embodiment 173, wherein the nanopore device to read data is the same nanopore device to write data.

Embodiment 175. The method of embodiment 174, wherein the encoded data is read by stretching and imaging the generated polymer.

### Examples

Described herein are various examples of compositions, systems, and methods for data storage utilizing polymers. Examples of writable nucleic acid polymers, methods to produce such polymers, methods to writing data, and methods for reading data are provided.

### Example 1: Writing Data in DNA or ROMP Polymer with Light Pulses Using a Plasmonic Nanopore

A repeating DNA strand or ROMP polymer with modifiable fluorophore bits is constructed to contain in each repeating unit two spectrally distinguishable caged dyes. The dyes chosen are the coumarin shown in FIG. 2C and the Tokyo green caged dye in FIG. 2A.

The two dyes are placed close to one another in the repeating unit, such that irradiation results in both dyes being illuminated at the same time. This unwritten (“blank”) DNA or ROMP polymer is placed into a nanopore apparatus further comprising a gold plasmonic bowtie at the pore exit. The DNA or ROMP polymer is placed in a buffer containing sodium chloride at 500 mM on the dark side of the nanopore. A voltage is applied via electrodes placed into the solution on either side of the pore, providing impetus for the DNA or ROMP polymer to translocate through the nanopore. As the unwritten DNA or ROMP polymer passes though the pore, lasers or LED lights are impinged on the bowtie structure at the pore exit in pulses. A long wavelength (455 nm) pulse of 10 milliseconds results in uncaging of the coumarin dye, resulting in a “zero” bit being written, which can later be identified by its blue fluorescence. As the DNA passes through further, a pulse of light at 365 nm is impinged on the bowtie structure, resulting in uncaging of the green dye, resulting in having written a “one” bit, which can later be identified by its green fluorescence. In this way a series of pulses of light writes binary data into the DNA strand as it passes through the pore. Writing efficiency and speed can be tuned by altering the rate at which the DNA or ROMP polymer passes through, and by the length and intensity of the light pulses.

### Example 2: Writing Data in an Organic Polymer by Photoconversion

A polyacrylic acid (PAA) polymer containing heptamethine dye in each repeating monomer is used to write data in the strand using a nanopore apparatus further comprising a gold plasmonic bowtie at the pore exit. The PAA polymer is placed in a buffer containing sodium chloride at 500 mM on the dark side of the nanopore. A voltage is applied via electrodes placed into the solution on either side of the pore, providing impetus for the DNA to translocate through the nanopore. As the unwritten polymer strand passes though the pore, lasers or LED lights are impinged on the bowtie structure at the pore exit in pulses. A long wavelength (740 nm) pulse results in loss of two methine units, resulting in a pentamethine dye, which is a “zero” bit being written, which can later be identified by its red fluorescence.

As the polymer passes through further, a pulse of dual wavelength light at 740 and 638 nm is impinged on the bowtie structure, resulting in conversion of a heptamethine dye to a trimethine dye as a result of two-step loss of two methine units and an additional two methine units. This results in having written a “one” bit, which can later be identified by its green/yellow fluorescence. In this way a series of pulses of light writes binary data into the polymer strand as it passes through the pore. Writing efficiency and speed can be tuned by altering the rate at which the polymer passes through, and by the length and intensity of the light pulses.

### Example 3: Writing Data in DNA by Light-Triggered Release of Quenchers and Cages

A repeating DNA strand with writable bits is placed into a nanopore apparatus further comprising a gold plasmonic bowtie at the pore exit. The DNA is placed in a buffer containing sodium chloride at 500 mM on the dark side of the nanopore. A voltage is applied via electrodes placed into the solution on either side of the pore, providing impetus for the DNA to translocate through the nanopore. As the unwritten DNA passes though the pore, lasers or LED lights are impinged on the bowtie structure at the pore exit in pulses. A long wavelength (430 nm) pulse of 10 milliseconds results in loss of the quencher, resulting in a “zero” bit being written, which can later be identified by its blue fluorescence. As the DNA passes through further, a pulse of light at 365 nm is impinged on the bowtie structure, resulting in loss of a quencher and uncaging the green dye, resulting in having written a “one” bit, which can later be identified by its green/blue fluorescence. In this way a series of pulses of light writes binary data into the DNA strand as it passes through the pore. Writing efficiency and speed can be tuned by altering the rate at which the DNA passes through, and by the length and intensity of the light pulses.

### Example 4: Reading Written DNA Polymers Via Nanopore Sequencing

Common nanopore sequencing devices measure current flow of electrolytes during passage of a DNA molecule through the pore. Since DNA bases each differ in size and shape, this slightly alters the current as each different base passes the pore. In this example, an experiment is carried out with a commercial nanopore device, and the readout is changes in current over time while a written DNA tape passes through. The “1” and “0” bits comprise G and nitrobenzylG, which differ considerably in size. Experiments with DNA tapes having bits in all-“0” state (blank polymer) reveal the lowering of current when the largest nitrobenzylG nucleotides pass through, and can distinguish the differences in current between these “0” bits and the spacers and delimiters. Separately, DNA all-“1” polymers are measured, showing the level of current observed as the “1” (G) bits pass though. These experiments provide calibration for reading and distinguishing current levels that denote “1” and “0” bits. Next, fully written DNA polymers are passed though. Current levels denoting “1” and “0” are read and placed in context of current levels seen for spacers and delimiters. Multiple reads of the same strand are used, if needed, to improve accuracy of data reading.

### Example 5: Reading Data in DNA by Nanopore Current Flow

The written DNA polymer from Example 1 is placed into a plasmonic nanopore device. The DNA is placed in a buffer containing sodium chloride at 500 mM on the dark side of the nanopore. A voltage is applied via electrodes placed into the solution on either side of the pore, providing impetus for the DNA to translocate through the nanopore. Light is impinged onto the bowtie structure continuously at 365 nm, providing excitation of the non-dark dyes as they pass though. The device further comprises an imaging sensor on the light side of the device. Where only coumarin is uncaged, a blue dye is observed. Where both dyes are uncaged, a green signal is observed due to Foerster resonance energy transfer from the coumarin to Tokyo Green. Microscopic imaging in continuous mode results in a string of blue and green signals, and software converts this string into binary code.

### Example 6: Installing Writable Bits onto Biologically Derived DNA

In this example, a 48 kbp double-stranded genomic DNA from bacteriophage lambda is used for installing sets of chemically modifiable structures. The DNA is dissolved in aqueous solution containing DMSO, which can help dissolve photoconvertible heptamethine dye to be utilized as chemically modifiable structures, (FIG. 8B). The bit heptamethine dye module is functionalized with a mesylate group, rendering it reactive with guanine in double-stranded DNA. The bit module is dissolved at 1 mM in solution with the DNA and incubated for 48 h at 37° C. to maximize the number of installed adducts. The DNA is then isolated by precipitation. Subsequent analysis reveals that the majority of guanine nucleotides are alkylated by the bit module, yielding a writable polymer with iteratively spaced photoconvertible heptamethine dye.

### Example 7: Polymerase Extension to Incorporate Reactive Groups into Nucleic Acid Polymers

In this example, a linear 48 kbp double-stranded genomic DNA from bacteriophage lambda is dissolved in buffer, denatured by heat (95° C., 10 min), and then rapidly cooled on ice, yielding two separated single strands of the genomic DNA. The ssDNA strands are mixed with 25nt DNA primer with sequence complementary to the 3′ ends of one of the genomic strands in a solution at 25° C. containing a Mg2+-containing buffer supportive of hybridization. BST DNA polymerase is added with three standard dNTPs (dATP, dCTP, dGTP) and aminopropynyl-dUTP (S. E. Lee, et al., Nucleic Acids Res. 2001; 29:1565-73, the disclosure of which is incorporated herein by reference), at 100 uM each, to the solution. The DNA and polymerase solution is incubated at 65° C. for several hours, resulting in the polymerase synthesizing a complete copy of the first strand with amine functional groups at T positions. This DNA duplex is nucleophilic at these amine positions and can react with electrophiles. A solution of a bit module (i.e., containing two chemically alterable groups) carrying a reactive NHS ester group is then incubated in molar excess over the number of amine groups in the solution in order to install the bits. Sufficient DMSO is present to dissolve the bit module and keep the DNA dissolved. Extended incubation results in double-stranded DNA in which the majority of amine groups are acylated with a bit module. This writable DNA is isolated from excess reagent and from single stranded DNA.

### Example 8: Incorporation of Phosphorothioate Groups into DNA and Installation of Bit Modules

In this example, a linear 48 kbp double-stranded genomic DNA from bacteriophage lambda is dissolved in buffer, denatured by heat (95° C., 10 min), and then rapidly cooled on ice, yielding two separated single strands of the genomic DNA. The ssDNA strands are mixed with 25nt DNA primer with sequence complementary to the 3′ ends of one of the genomic strands in a solution at 25° C. containing a Mg2+-containing buffer supportive of hybridization. BST DNA polymerase is added with three standard dNTPs (dTTP, dCTP, dGTP) and dATP-alpha-S, at 100 uM each, to the solution. The DNA and polymerase solution is incubated at 65° C. for several hours, resulting in the polymerase synthesizing a complete copy of the strand with phosphorothioate linkages at A positions. This DNA duplex is nucleophilic at these phosphorothioate positions and can react with electrophiles. A bit module (i.e., a set of chemically alterable groups) carrying a reactive iodoacetyl group is then incubated in molar excess over the number of thioate groups in the reaction. Sufficient DMSO is present to dissolve the bit module and keep the DNA dissolved. Extended incubation results in double-stranded DNA in which the majority of thioate groups are alkylated with a bit module. This writable DNA is isolated from excess reagent and from single stranded DNA.

### Example 9: Data Encoding into Nucleic Acids Via Stable and Alterable Secondary Structures

In this example, synthetic DNA oligonucleotides are constructed with sequences as shown in FIG. 12, but with an additional four nucleotides (TCGA) added to the 5′ end of each strand as a ligatable end, along with terminal 5′-phosphate groups. Modified deoxyuridine monomers are incorporated into the “top” strand during DNA synthesis (see examples in FIG. 13), yielding pairs of modified deoxyuridine monomers with reactive functional groups. Each of these two strands (the modified one and the unmodified complement) is separately ligated with T4 DNA ligase and ATP into a long repeating polymer using a short “splint” oligonucleotide to bring ends together. At least one of each pair of modified deoxyuridine monomers contains a photocleavable linker. Examples of such reactive groups are found in FIG. 10. The single-stranded folded modified-strand polymer in an aqueous buffer containing salts to promote folding is reacted to form stable and alterable linkages between X and Y and separately between Z and Q, promoted by their proximity in the folded hairpins (see FIG. 12). These stable bonds trap the DNA in the folded state, and destabilize simple duplexes. As a result, hybridization with the complementary DNA polymer maintains dual hairpin structures. To encode data into the polymer, as the polymer passes a light energy source and in accordance with a digital code to be encoded, one or more pulses of violet light energy (e.g., approximately 430 nm) is provided at designated locations of the polymer to cleave a functional group of the Z and Q pair, resulting in an “unlocking” of the link between the Z and Q, and alteration of the hairpin formation to a simple duplex. Further, one or more pulses of UV light energy (e.g., approximately 365 nm) is provided at designated locations of the polymer to cleave a functional group from both the Z and Q pair and the X and Y pair, resulting in an unlocking both links, and alteration of both hairpin formations to a simple duplex. These three different DNA conformations (small and large hairpin, simple duplex) are distinguishable by nanopore sequencing platforms via differences in current flow as the structures pass through the pore.

### Example 10: Writable Bit Incorporation into an Organic Polymer with a Photoconvertible Fluorophore

In this example, a polymer prepared by ring-opening metathesis polymerization (ROMP) utilizing a norbomene-imide (S. Sutthasupa, M. Shiotsuki, and F. Sanda, Polym J. 2010; 42:905-915, the disclosure of which is incorporated herein by reference). The nitrogen of the imide is substituted by an azidobutyl group prior to polymerization, resulting in the polymer having azide sidechain groups at each monomer. The polymer is dissolved in dimethylformamide (DMF). Photoconvertible heptamethine dye bit modules with a terminal alkyne group at position “X” (see FIG. 8B) are added to the DMF solution in a molar excess of the azide sidechain groups. To carry out a CuAAc reaction, copper (I) and tris(triazolyl)methyl amine ligand are added to catalyze the reaction, and the solution incubated with agitation at room temperature for 2 days in the dark. The functionalized polymer is isolated, and subsequent analysis reveals that the majority of azide sidechains are modified with a photoconvertible heptamethine dye bit module.

### Example 11: Writable Bit Incorporation into a Peptide Polymer

In this example, a polymeric peptide comprising the repeating sequence Lys-Ala is used to generate a data-writable polymer. The peptide is dissolved in dimethylformamide (DMF). Bit modules comprising a quencher, a blue coumarin fluorophore, and a caged Tokyo green fluorophore, and functionalized with a carboxylate group at position “X” (see FIG. 8C) are added to the DMF. The peptide coupling agent EDC is added, and the solution incubated with agitation at room temperature for 2 days in the dark. The polymeric functionalized peptide is isolated, and subsequent analysis reveals that the majority of lysine sidechains are modified with a bit module comprising the quencher, the blue coumarin fluorophore, and the caged Tokyo green fluorophore.

### Example 12: Preparation and Localized Photobleaching of Cy-5-Labelled DNA

This example demonstrates stretching and writing a synthetic DNA with fluorophores (Cy-5) labelled.

In this example, the following sequence (SEQ ID NO: 1) was used:

ATTCGCGGCCGCAGGCTAGGTGGAGGCTCAGTGATGATAAGTCTG
CGATGGTGGATGCATGTGTCATGGTCATAGCTGTTTCCTGTGTG
AAATTGTTATCCGCTCAGAGGGCACAATCCTATTCCGCGCTATCC
GACAATCTCCAAGACATTAGGTGGAGTTCAGTTCGGCGTATGGCA
TATGTCGCTGGAAAGAACATGTGAGCAAAAGGCCAGCAAAAGGCC
AGGAACCGTAAAAAGGCCGCGTTGCTGGCGTTTTTCCATAGGCTC
CGCCCCCCTGACGAGCATCACAAAAATCGACGCTCAAGTCAGAGG
TGGCGAAACCCGACAGGACTATAAAGATACCAGGCGTTTCCCCCT
GGAAGCTCCCTCGTGCGCTCTCCTGTTCCGACCCTGCCGCTTACC
GGATACCTGTCCGCCTTTCTCCCTTCGGGAAGCGTGGCGCTTTCT
CATAGCTCACGCTGTAGGTATCTCAGTTCGGTGTAGGTCGTTCGC
TCCAAGCTGGGCTGTGTGCACGAACCCCCCGTTCAGCCCGACCGC
TGCGCCTTATCCGGTAACTATCGTCTTGAGTCCAACCCGGTAAGA
CACGACTTATCGCCACTGGCAGCAGCCACTGGTAACAGGATTAGC
AGAGCGAGGTATGTAGGCGGTGCTACAGAGTTCTTGAAGTGGTGG
CCTAACTACGGCTACACTAGAAGAACAGTATTTGGTATCTGCGCT
CTGCTGAAGCCAGTTACCTTCGGAAAAAGAGTTGGTAGCTCTTGA
TCCGGCAAACAAACCACCGCTGGTAGCGGTGGTTTTTTTGTTTGC
AAGCAGCAGATTACGCGCAGAAAAAAAGGATCTCAAGAAGATCCT
TTGATCTTTTCTACGGGGTCTGACGCTCTATTCAACAAAGCCGCC
GTCCCGTCAAGTCAGCGTAAATGGGTAGGGGGCTTCAAATCGTCC
GCTCTGCCAGTGTTACAACCAATTAACAAATTCTGATTAGAAAAA
CTCATCGAGCATCAAATGAAACTGCAATTTATTCATATCAGGATT
ATCAATACCATATTTTTGAAAAAGCCGTTTCTGTAATGAAGGAGA
AAACTCACCGAGGCAGTTCCATAGGATGGCAAGATCCTGGTATCG
GTCTGCGATTCCGACTCGTCCAACATCAATACAACCTATTAATTT
CCCCTCGTCAAAAATAAGGTTATCAAGTGAGAAATCACCATGAGT
GACGACTGAATCCGGTGAGAATGGCAAAAGCTTATGCATTTCTTT
CCAGACTTGTTCAACAGGCCAGCCATTACGCTCGTCATCAAAATC
ACTCGCATCAACCAAACCGTTATTCATTCGTGATTGCGCCTGAGC
GAGACGAAATACGCGATCGCTGTTAAAAGGACAATTACAAACAGG
AATCGAATGCAACCGGCGCAGGAACACTGCCAGCGCATCAACAAT
ATTTTCACCTGAATCAGGATATTCTTCTAATACCTGGAATGCTGT
TTTCCCGGGGATCGCAGTGGTGAGTAACCATGCATCATCAGGAGT
ACGGATAAAATGCTTGATGGTCGGAAGAGGCATAAATTCCGTCAG
CCAGTTTAGTCTGACCATCTCATCTGTAACATCATTGGCAACGCT
ACCTTTGCCATGTTTCAGAAACAACTCTGGCGCATCGGGCTTCCC
ATACAATCGATAGATTGTCGCACCTGATTGCCCGACATTATCGCG
AGCCCATTTATACCCATATAAATCAGCATCCATGTTGGAATTTAA
TCGCGGCCTCGAGCAAGACGTTTCCCGTTGAATATGGCTCATAAC
ACCCCTTGTATTACTGTTTATGTAAGCAGACAGTTTTATTGTTCA
TGATGATATATTTTTATCTTGTGCAATGTAACATCAGAGATTTTG
AGACACAACGTGGCTTTCCCCCGCCGCTCTAGAACTAGTGGATCC
AAATAAAACGAAAGGCTCAGTCGAAAGACTGGGCCTTTCGTTTTA
TCTGTTGTTTGTCGCATTATACGAGACGTCCAGGTTGGGATACCT
GAAACAAAACCCATCGTACGGCCAAGGAAGTCTCCAATAACTGTG
ATCCACCACAAGCGCCAGGGTTTTCCCAGTCACGACGTTGTAAAA
CGACGGCCAGTCATGCATAATCCGCACGCATCTGGAATAAGGAAG
TGCCATTCCGCCTGACCTTGCCTCTCGCCAGCATTGTAATAGGCC
TCAGCTACCTGGGTACCTGAATCCATCGCTGCGTAGCTGGATAGG
TGCGTGACTAAATGAGTAAGTGAGTCGCTACATCCCTACATCCGT
CGCTGGCTAACTCACTGCGTAACTCCATGGCTGAATGAGTCGATA
GCTCGCTGAATGAGTACGTGAATCAGTAAGTACGTAGATCGGTGG
CTGCGTACCTGAATCCCTCAATGAATCACTAGGTCACTAACTCAA
TACATGCCTAGGTAGGTAAGTAAGTCAATGACTGGCTGGCTGAAT
CGATAACTGCATAGCTACATCAGTAGATACATAAGTGAATGAGTC
CGTAGATGGGTAACTCCATGAATCACTGCCTGAATCAGTCAATCA
CTACGTAGGTAACTAGATAAATCGGTGAATACGTGCGTGAATGGA
TAGGTAGATAGGTAGCTGGATCCGTCCCTAACTCGCTCAGTGCGT
AGATCACTGAATCAGTCGATCCGTACGTACCTCAGTCGATACGTG
AGTAGCTAGGTCGCTCGCTCAATGGGTAAATACATACATCACTGC
CTGCGTGAGTCCGTGCATCGATAAGTGGATGGCTAAGTGGCTGAG
TAACTCAATGAATACATACGTGAGTCGCTGCCTAGATGCATCGCT
ACCTGAATGAATACCTGCGTGGGTCAGTGCCTAGCTGGGTCAGTA
ACTCGATGAGTCCCTAGATCCGTCACTCAGTAGCTGAATGCATCG
CTCGCTAGGTAACTCCCTCGATACATCCATAGATACGTCACTGGC
TGGATCACTCGATGAATGCATCACTCAGTCAATCCATCGCTCACT
CGCTCCCTGGGTGACTCAATGCGTCAGTGAATAAGTGACTCGCTG
GATGGATGCATCAGTCAGTGCATCACTAAATGGGTAGATACGTAC
CTACCTACATCAGTCCATCACTCACTACCTAAATGAGTGGCTGGA
TCGGTCGGTCACTAAGTACGTACCTCCCTAACTCACTCGGTGCCT
AACTCGATAGGTACCTAAATCAGTGAATACGTGCCTCGGTAGCTA
GATAAGTGCCTCGATGAATACGTAACTAGATACATCCGTGGGTGG
CTCCGTGGATCGGTGAGTGGGTCGATGCCTCACTGCCTGAGTAAA
TGCCTCACTGGCTGCCTACGTGACTACATCCCTGCGTGGCTCACT
CACTCGGTGCGTAGATCAGTCCCTGGCTGCGTCCCTGCGTGCCTG
GCTGAATAGGTGGATGCGTACATCAGTACATCCCTGCATGGCTCG
GTGCATACCTGACTCCGTCACTGGCTCAGTACCTCGATAAGTGAC
TCGGTCAATCAGTGCGTGGGTCCATCAATCAATCACTGGCTCCCT
CAGTCCATCAATACATGGCTGAGTGAATACATACCTGGATGCATC
ACTGGGTGGCTGGCTGGATACCTAAGTACGTAGGTGCCTACATAA
GTAACTAAGTCGCTGCATCGATCCGTACCTGCGTCAATGGCTAGG
TCCGTCCGTAAGTCGATACGTGAGTCCATGGATGCATACATCGAT
ACGTCCATAGATCGATACGTGCATACATAAGTCGCTACGTGAGTA
ACTACCTACATGAATACATCCATCCATGCGTGACTAAGTAGATCC
GTGGCTGGATCACTACATCGATCGCTGCGTGCGTGCGTCACTAGC
TCAGTGGATACGTGAATGAATGAATACATCGGTCACTCGCTAGGT
GCATAAGTCGATGAATAGGTGCATACGTGAGTAACTAAATGCCTC
GCTGGATGAATCACTGGCTGAATAGATAACTGCGTAGGTGGGTAG
GTAGCTCCATCGGTGGCTCCATGGGTGAATCCCTAACTGGATGGG
TGCCTACCTAACTGAGTAGCTGGGTCGATAAATCGATCCCTCCGT
AACTAACTACATCGCTGCGTACCTGCGTGGATCGCTGAATCGGTC
CCTGAATCGATCACTAGATAAATACCTCGATCGCTAAATGGCTGC
CTGGATAGGTGAATAGCTAGGTGGGTACCTCACTAGATGCATAGC
TCCATGACTCCCTCCATCGATACGTCGGTGACTCCCTGGCTAGGT
GCCTAGGTGACTCCATACGTGCGTAGCTGGATGACTCGGTGCGTC
CATCGGTGGGTGGCTAAGTCGCTACATCCGTGGGTGCCTCGATCC
ATCCCTGGATCCATAAGTCCATACGTGCATACATGACTACCTAGC
TCGGTGGATACATCGCTCCATGACTGCATCAGTACGTAAATAACT
CGGTAGATGCATAGATACATGGCTGCGTGACTCGATCAATGAATA
GGTCAATAGCTCCGTCGCTGCCTCGCTAGCTGGGTGGATGACTAC
GTGGATACATAAGTGGATGAATGGCTGGCTGGATCCCTCCCTCCA
TCACTGGCTACCTAAGTGAATAACTACCTAACTAGCTAGATGGCT
GGGTCCATCAATGGCTAGATCCCTCAATAGGTACATCGGTCCATG
CATCCATAACTACGTCAGTCCATACGTCCATCCATGAGTCGATCG
ATGGGTAGGTACATACGTAAATGCGTAAGTCAATCCCTACATGCC
TACATCACTAGGTGGATGAGTCGGTGGATGCGTAGCTGCATCCCT
AGGTACATCACTCCGTGAGTAAATACCTACATGGCTAGGTAAATC
CATAAGTCGGTCACTCGCTCCGTCCCTGCGTCCCTGCCTAAGTAG
ATGAATAGGTCACTGCATGCCTAGCTAACTAGATCCCTAAGTACA
TGAGTAAATCAATCCCTGCATGCCTCGGTGCATACATAGGTGAGT
GAATGAGTCGCTACGTAACTCACTCAATCGATAAGTCAGTACCTC
CCTGCCTACCTGACTAGGTGCGTAAATAGATAAATACGTGGCTCC
ATACATAAGTAGGTGGCTAACTCACTAGCTGGATGGCTCAATCGG
TGCCTCGCTAGCTCGCTGCGTAACTCGGTAAATCACTAGCTAACT
GCGTCCCTGAGTAGCTCCGTAACTAGCTAAGTGCCTAACTCAGTG
CGTAGCTAACTAACTAGCTCGCTGAGTCACTGCATCAATCAATGC
ATAACTGGGTGAATAAATCAATACCTCCATGAGTAGATAAATAAG
TGCGTCGGTGAATGCCTGAGTGACTCGATCAGTCAGTGCGTGGCT
AGGTGGGTCGGTCCGTCACTGGCTCGCTACGTGGGTGACTGCCTG
CCTCCGTACGTACCTGCGTAGGTACATAGATACCTCAGTAACTCG
CTGCCTCGCTAGCTGGGTACGTGGATAACTAGATCGGTAGGTCCA
TGAATCAATACGTACATCCCTCAATACGTGAGTAAATCCCTCACT
CGCTAAATGGCTGACTACGTCCGTCCGTGACTAAGTAGGTAAGTC
GCTGGATAACTCAATACCTGGCTACATAGCTCAGTCGGTACCTAA
ATGCCTCAATGGCTCGCTGAGTCGGTCAATCCCTCCATCCGTAAC
TCACTAAGTCAGTCCATGCCTAGGTAACTCCCTCGATAACTAACT
GGGTAAATGACTAACTCGCTACGTAAATGCCTGCGTACGTCAATA
GATCACTCCCTGAATGGGTGGCTGGCTGGGTCGCTAGCTCCATCC
ATACATGGCTCCGTGCGTCGCTACATCAATGCGTAGCTCGGTCAA
TGCCTGCGTACGTCGATAAATAGCTGCCTACGTGAGTAGATGGGT
CCCTCAATCCCTGAATAAGTACGTAAGTCCGTACCTCAATACCTA
ACTGCCTGCATCAATACCTAGATAAGTGCGTGACTGAATAGGTCA
GTCGATCGCTAGGTCAATGAGTGGCTCGATGCATGGGTAGATAGA
TGCGTGGATAGATACGTGCCTGGCTGAATAGGTAGATGCCTACAT
CCATGACTCGCTCGGTCGGTCACTCAATGGATAAATGACTAAGTA
CGTCGCTGCATCACTCCATCCGTCCCTAAGTAGGTGGCTACGTCA
GTCGATGAGTGGGTGAGTAGATCGATAACTGAATAGCTCAATAAA
TGGGTCCATGGCTGGCTGGATGCGTCGGTGAGTGCATGGGTCCGT
ACATCGGTCACTCCCTCGATGGCTCCATCGGTCCATGCCTGGCTC
GATACGTGAATAGATACATAGCTGACTGGGTCACTAAGTGAGTGC
GTCCATGCATCCCTACGTACGTGAGTCAGTACGTCAATGCGTAAC
TACGTGCATGAATAACTCGATCCGTCAGTGCCTAAGTGAATAGCT
GGCTAACTGGATCACTGCATAGCTCCATCCCTGGCTCCATGGATG
AATCGCTGCATCCATAACTCGATCCGTACGTACGTCAATAGGTGA
CTCCCTGAGTCAGTAGATGGCTACGTCGATGCCTAGATAGGTGGA
TGCGTACCTCGATAGGTAACTACGTAAGTCGATCGATCCATCCGT
GCCTGCGTGGATCGGTAGATAACTAGCTACATCCCTCCATGCATA
CATACATAAATGAATGACTCGCTCCATCACTGCGTGACTGCATAC
GTGAATAAATACGTGCGTGCCTCGATACATGCGTGACTCAATGAC
TCGCTCCGTCCGTGCGTCCGTAACTCGGTAAATCGGTACATGCCT
GCATACATCAGTGCCTCCGTCCCTGCATACGTCCGTCCATCGATC
GATAGCTGGCTAAATAGGTGGGTAGGTAGATGCCTGCGTCGATAG
ATGAATACATCGGTAAATGGCTCGCTACGTAAGTAGGTGGGTAGA
TCAGTGGGTACGTCGCTAGCTAGATAGGTCGATACATAACTGACT
CACTCGGTCCGTACGTGGGTAAATCAATAGCTAGCTACCTGCGTA
CCTGCATCGATGGCTAAATCCATACGTCCATAGATCACTCCCTGC
ATCGGTGAATGGGTGAATCAATCAGTGCGTCCCTGGGTACGTGGC
TAAATAACTAGATGGCTGCATAGGTAACTGCATCGATACATCACT
ACGTACATCCATAAGTCCGTCCATAAGTCGATCGCTAGATAAATG
AGTGCGTAAATGAGTGCCTCGGTGGATAGATGCGTACCTCGATAG
GTGCGTGGCTACCTCGGTACGTCCGTCCCTAGATGGCTAGGTGCA
TAACTGGCTACGTAACTCGCTCCGTCGGTGCCTACCTAAATAAGT
CCGTGCGTCCCTGCATCCATCCCTAAATCGATAAGTCGCTAGCTC
GATCAGTAAGTACATCCCTGAATCAATAAGTAAATCCCTCCGTAC
GTGAGTGAGTGCGTAGGTGCATCAGTGGATAAGTGGGTGCATCAC
TGCGTCCATGCATCACTACCTAAGTCCGTGCGTAGGTAGATCAGT
ACGTGAGTGCCTAAGTGCGTAGCTACGTAAGTGAGTGACTGAGTG
CCTGGGTCCATGAATACCTCCATACGTGGGTACCTACGTCGGTGA
CTAGGTCACTGCATACCTGGGTCCGTAGGTACCTAGCTGCATAGA
TAAGTCCCTGCGTGGATGCCTCCGTGGCTGAATCGGTCAATGCAT
GGATAAATACGTGCATCGCTGAATGGCTCAGTCAATACATCAGTG
G.

### DNA Preparation Using Plasmid

DNA with the above sequence (SEQ ID NO:1) was prepared using a plasmid and a DNA polymerase by replacing T's with dye-labelled dUTPs (such as Cy5-dUTP), affording DNAs with labeled U's, e.g. (where N may be A or C or G): . . . NNNNNNNNUNNNUNNNUNNNUNNN . . .

### DNA Clean Up

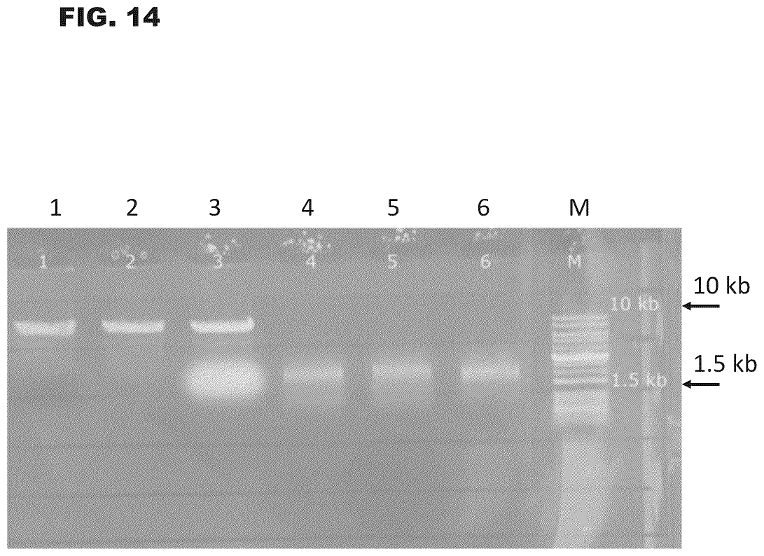

The reaction mixture containing the prepared DNA was mixed with 100 μL of CleanNGS DNA & RNA Clean-Up Magnetic Beads, incubated at room temperature for 5 minutes, and left on magnetic stand for 3 minutes. The clear liquids were aspirated and discarded. The magnetic beads were washed with 400 μL of 70% Ethanol 2 times with one minute incubation each time. After ethanol wash, the beads were left dried for one minute and then incubated with 50 uL IDTE buffer for 5 minutes on a rack. The tube was then moved back to the magnetic stand and left for 3 minutes. After all the magnetic beads moved to the side, the clear liquid was transferred to a new 1.5 mL Eppendorf tube. The concentration of DNA was measured using Qubit fluorometer and the integrity was checked using gel electrophoresis. Typically, 500-1000 ng labeled DNA was recovered. An example of labeled DNA electrophoresis can be seen in FIG. 14, where Lane 1 contains Cy5-labeled DNA and Lane M contains Safegreen 1 kb DNA ladder.

### DNA Combing and Stretching

Materials for the DNA combing protocol included: The Fibercomb instrument, genomic DNA extraction kit, stretching reservoirs and vinylsilane coated coverslips purchased from Genome Vision. Prolong Gold mounting solution was from Thermofisher, and YOYO-1 was from Biotium.

In the DNA stretching protocol, 1 μL of labeled DNA (20-50 ng) was diluted in 2.4 mL of Buffer 6 from the genomic DNA extraction kit. The solution was warmed to 37° C. for 5 minutes and then loaded into the reservoir. The vinyl-silane coated coverslips were incubated in the reservoir for 3 minutes and pulled up slowly, causing DNA to stretch on the coverslip using the Fibercomb instrument. The coverslips were heated to 125° C. for 3 minutes and then mounted on a slide with 10 μL of Prolong Gold mounting solution with 1 uM YOYO-1. The coverslips were sealed with nail polish.

### DNA Bleaching and Imaging

Stretched DNA on coverslips were imaged using a Cytiva OMX-SR microscope. The slide with stretched DNA was loaded on the microscope per manufacturer's instructions. A rectangle box perpendicular to the stretched DNA was used to bleach segments of the Cy5 labeled DNA for 25 milliseconds using a 633 nm laser.

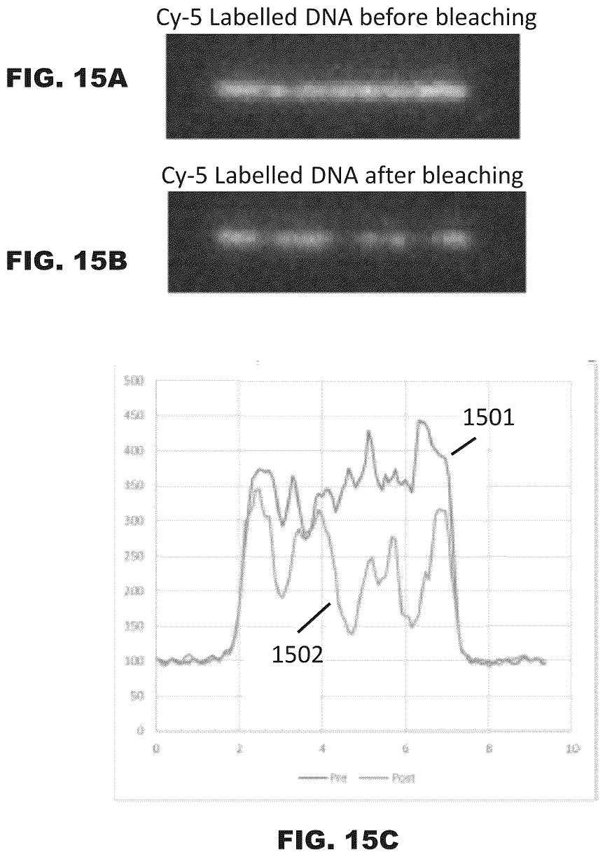

The prepared DNA with Cy-5 labelled Us was locally photobleached with light to write on the DNA. Results for DNA imaging and bleaching are shown in FIGS. 15A-15C. Y axis shows emission intensity, and X axis is distance (microns).

FIG. 15A shows an image of Cy5 labeled DNA before bleaching and FIG. 15B shows an image of Cy5 labeled DNA after sequential photobleaching. FIG. 15C shows a plot displaying two cross section profiles: before bleaching (1501) and after bleaching (1502), showing three distinct sites of bleaching, observable in darkened spots in the DNA and as valleys in the intensity plot. The peak to valley distance is about 700 nm. From FIG. 15C the separation of the pre-bleached state represented by the pre-bleach profile (1501) and post bleached state represented by the post-bleach profile (1502) can be clearly seen.

1-167. (canceled)

168. A polymer, comprising:
(a) a plurality of convertible residues, wherein each convertible residue of said plurality of convertible residues is in a first state and is capable of being converted into a second state and comprises:
(i) a fluorophore; and
(ii) a separate leaving group; and
(b) a plurality of spacer residues;
wherein said polymer comprises at least 10 convertible residues.

169. The polymer of claim 168, wherein said plurality of spacer residues comprises nucleobases.

170. The polymer of claim 169, wherein said nucleobases are naturally occurring nucleobases.

171. The polymer of claim 168, wherein said separate leaving group impairs fluorescence of said fluorophore.

172. The polymer of claim 168, wherein said separate leaving group is selected from and

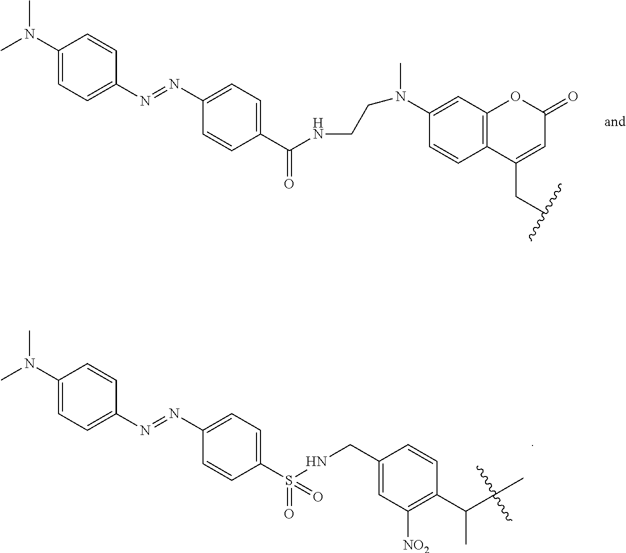 [SMILES: *Cc1cc(=O)oc2cc(N(C)CCNC(=O)c3ccc(/N=N/c4ccc(N(C)C)cc4)cc3)ccc12 ; *C(C)c1ccc(CNS(=O)(=O)c2ccc(/N=N/c3ccc(N(C)C)cc3)cc2)cc1[N+](=O)[O-]]

173. The polymer of claim 168, wherein said separate leaving group cages said fluorophore.

174. The polymer of claim 168, wherein said polymer comprises a nucleic acid.

175. The polymer of claim 174, wherein said nucleic acid is a single-stranded nucleic acid.

176. The polymer of claim 174, wherein said nucleic acid is a double-stranded nucleic acid.

177. The polymer of claim 174, wherein said nucleic acid comprises a nucleobase and said fluorophore is attached to said nucleobase.

178. The polymer of claim 168, wherein said polymer comprises at least 100 convertible residues.

179. The polymer of claim 178, wherein said polymer comprises at least 1,000 convertible residues.

180. The polymer of claim 168, wherein a ratio of convertible residues of said plurality of convertible residues to spacer residues of said plurality of spacer residues is between 2 to 1000.

181. The polymer of claim 180, wherein a ratio of convertible residues of said plurality of convertible residues to spacer residues of said plurality of spacer residues is between 2 to 100.

182. The polymer of claim 168, wherein said first state of each of said convertible residues of said plurality of convertible residues has a first emission wavelength and said second state of each of said convertible residues of said plurality of convertible residues has a second emission wavelength.

183. The polymer of claim 168, wherein said second state comprises a naturally occurring residue.

184. The polymer of claim 168, wherein spacer residues of said plurality of spacer residues are identical.

185. The polymer of claim 168, wherein said plurality of spacer residues comprise a non-nucleic acid residue.

186. The polymer of claim 185, wherein said non-nucleic acid residue comprises an ethylene glycol residue.

187. The polymer of claim 168, wherein said fluorophore is attached to said separate leaving group.


SEQUENCE LISTING (1 sequence)

SEQ ID NO: 1 - DNA, 7740 bp, synthetic construct
  source 1..7740: mol_type = other DNA
attcgcggccgcaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgaccttgcctctcgccagcattgtaataggcctcagctacctgggtacctgaatccatcgctgcgtagctggataggtgcgtgactaaatgagtaagtgagtcgctacatccctacatccgtcgctggctaactcactgcgtaactccatggctgaatgagtcgatagctcgctgaatgagtacgtgaatcagtaagtacgtagatcggtggctgcgtacctgaatccctcaatgaatcactaggtcactaactcaatacatgcctaggtaggtaagtaagtcaatgactggctggctgaatcgataactgcatagctacatcagtagatacataagtgaatgagtccgtagatgggtaactccatgaatcactgcctgaatcagtcaatcactacgtaggtaactagataaatcggtgaatacgtgcgtgaatggataggtagataggtagctggatccgtccctaactcgctcagtgcgtagatcactgaatcagtcgatccgtacgtacctcagtcgatacgtgagtagctaggtcgctcgctcaatgggtaaatacatacatcactgcctgcgtgagtccgtgcatcgataagtggatggctaagtggctgagtaactcaatgaatacatacgtgagtcgctgcctagatgcatcgctacctgaatgaatacctgcgtgggtcagtgcctagctgggtcagtaactcgatgagtccctagatccgtcactcagtagctgaatgcatcgctcgctaggtaactccctcgatacatccatagatacgtcactggctggatcactcgatgaatgcatcactcagtcaatccatcgctcactcgctccctgggtgactcaatgcgtcagtgaataagtgactcgctggatggatgcatcagtcagtgcatcactaaatgggtagatacgtacctacctacatcagtccatcactcactacctaaatgagtggctggatcggtcggtcactaagtacgtacctccctaactcactcggtgcctaactcgataggtacctaaatcagtgaatacgtgcctcggtagctagataagtgcctcgatgaatacgtaactagatacatccgtgggtggctccgtggatcggtgagtgggtcgatgcctcactgcctgagtaaatgcctcactggctgcctacgtgactacatccctgcgtggctcactcactcggtgcgtagatcagtccctggctgcgtccctgcgtgcctggctgaataggtggatgcgtacatcagtacatccctgcatggctcggtgcatacctgactccgtcactggctcagtacctcgataagtgactcggtcaatcagtgcgtgggtccatcaatcaatcactggctccctcagtccatcaatacatggctgagtgaatacatacctggatgcatcactgggtggctggctggatacctaagtacgtaggtgcctacataagtaactaagtcgctgcatcgatccgtacctgcgtcaatggctaggtccgtccgtaagtcgatacgtgagtccatggatgcatacatcgatacgtccatagatcgatacgtgcatacataagtcgctacgtgagtaactacctacatgaatacatccatccatgcgtgactaagtagatccgtggctggatcactacatcgatcgctgcgtgcgtgcgtcactagctcagtggatacgtgaatgaatgaatacatcggtcactcgctaggtgcataagtcgatgaataggtgcatacgtgagtaactaaatgcctcgctggatgaatcactggctgaatagataactgcgtaggtgggtaggtagctccatcggtggctccatgggtgaatccctaactggatgggtgcctacctaactgagtagctgggtcgataaatcgatccctccgtaactaactacatcgctgcgtacctgcgtggatcgctgaatcggtccctgaatcgatcactagataaatacctcgatcgctaaatggctgcctggataggtgaatagctaggtgggtacctcactagatgcatagctccatgactccctccatcgatacgtcggtgactccctggctaggtgcctaggtgactccatacgtgcgtagctggatgactcggtgcgtccatcggtgggtggctaagtcgctacatccgtgggtgcctcgatccatccctggatccataagtccatacgtgcatacatgactacctagctcggtggatacatcgctccatgactgcatcagtacgtaaataactcggtagatgcatagatacatggctgcgtgactcgatcaatgaataggtcaatagctccgtcgctgcctcgctagctgggtggatgactacgtggatacataagtggatgaatggctggctggatccctccctccatcactggctacctaagtgaataactacctaactagctagatggctgggtccatcaatggctagatccctcaataggtacatcggtccatgcatccataactacgtcagtccatacgtccatccatgagtcgatcgatgggtaggtacatacgtaaatgcgtaagtcaatccctacatgcctacatcactaggtggatgagtcggtggatgcgtagctgcatccctaggtacatcactccgtgagtaaatacctacatggctaggtaaatccataagtcggtcactcgctccgtccctgcgtccctgcctaagtagatgaataggtcactgcatgcctagctaactagatccctaagtacatgagtaaatcaatccctgcatgcctcggtgcatacataggtgagtgaatgagtcgctacgtaactcactcaatcgataagtcagtacctccctgcctacctgactaggtgcgtaaatagataaatacgtggctccatacataagtaggtggctaactcactagctggatggctcaatcggtgcctcgctagctcgctgcgtaactcggtaaatcactagctaactgcgtccctgagtagctccgtaactagctaagtgcctaactcagtgcgtagctaactaactagctcgctgagtcactgcatcaatcaatgcataactgggtgaataaatcaatacctccatgagtagataaataagtgcgtcggtgaatgcctgagtgactcgatcagtcagtgcgtggctaggtgggtcggtccgtcactggctcgctacgtgggtgactgcctgcctccgtacgtacctgcgtaggtacatagatacctcagtaactcgctgcctcgctagctgggtacgtggataactagatcggtaggtccatgaatcaatacgtacatccctcaatacgtgagtaaatccctcactcgctaaatggctgactacgtccgtccgtgactaagtaggtaagtcgctggataactcaatacctggctacatagctcagtcggtacctaaatgcctcaatggctcgctgagtcggtcaatccctccatccgtaactcactaagtcagtccatgcctaggtaactccctcgataactaactgggtaaatgactaactcgctacgtaaatgcctgcgtacgtcaatagatcactccctgaatgggtggctggctgggtcgctagctccatccatacatggctccgtgcgtcgctacatcaatgcgtagctcggtcaatgcctgcgtacgtcgataaatagctgcctacgtgagtagatgggtccctcaatccctgaataagtacgtaagtccgtacctcaatacctaactgcctgcatcaatacctagataagtgcgtgactgaataggtcagtcgatcgctaggtcaatgagtggctcgatgcatgggtagatagatgcgtggatagatacgtgcctggctgaataggtagatgcctacatccatgactcgctcggtcggtcactcaatggataaatgactaagtacgtcgctgcatcactccatccgtccctaagtaggtggctacgtcagtcgatgagtgggtgagtagatcgataactgaatagctcaataaatgggtccatggctggctggatgcgtcggtgagtgcatgggtccgtacatcggtcactccctcgatggctccatcggtccatgcctggctcgatacgtgaatagatacatagctgactgggtcactaagtgagtgcgtccatgcatccctacgtacgtgagtcagtacgtcaatgcgtaactacgtgcatgaataactcgatccgtcagtgcctaagtgaatagctggctaactggatcactgcatagctccatccctggctccatggatgaatcgctgcatccataactcgatccgtacgtacgtcaataggtgactccctgagtcagtagatggctacgtcgatgcctagataggtggatgcgtacctcgataggtaactacgtaagtcgatcgatccatccgtgcctgcgtggatcggtagataactagctacatccctccatgcatacatacataaatgaatgactcgctccatcactgcgtgactgcatacgtgaataaatacgtgcgtgcctcgatacatgcgtgactcaatgactcgctccgtccgtgcgtccgtaactcggtaaatcggtacatgcctgcatacatcagtgcctccgtccctgcatacgtccgtccatcgatcgatagctggctaaataggtgggtaggtagatgcctgcgtcgatagatgaatacatcggtaaatggctcgctacgtaagtaggtgggtagatcagtgggtacgtcgctagctagataggtcgatacataactgactcactcggtccgtacgtgggtaaatcaatagctagctacctgcgtacctgcatcgatggctaaatccatacgtccatagatcactccctgcatcggtgaatgggtgaatcaatcagtgcgtccctgggtacgtggctaaataactagatggctgcataggtaactgcatcgatacatcactacgtacatccataagtccgtccataagtcgatcgctagataaatgagtgcgtaaatgagtgcctcggtggatagatgcgtacctcgataggtgcgtggctacctcggtacgtccgtccctagatggctaggtgcataactggctacgtaactcgctccgtcggtgcctacctaaataagtccgtgcgtccctgcatccatccctaaatcgataagtcgctagctcgatcagtaagtacatccctgaatcaataagtaaatccctccgtacgtgagtgagtgcgtaggtgcatcagtggataagtgggtgcatcactgcgtccatgcatcactacctaagtccgtgcgtaggtagatcagtacgtgagtgcctaagtgcgtagctacgtaagtgagtgactgagtgcctgggtccatgaatacctccatacgtgggtacctacgtcggtgactaggtcactgcatacctgggtccgtaggtacctagctgcatagataagtccctgcgtggatgcctccgtggctgaatcggtcaatgcatggataaatacgtgcatcgctgaatggctcagtcaatacatcagtgg